(19) United States
(12) Patent Application Publication
(10) Pub. No.: US 2005/0124562 A1
(43) Pub. Date: Jun. 9, 2005

(54) Bis-quinazoline compounds for the treatment of bacterial infections

(76) Inventors: Joseph Guiles, Lafayette, CO (US); Garry Dallmann, Boulder, CO (US); Nebojsa Janjic, Boulder, CO (US); Charles S. McHenry, Denver, CO (US); Xicheng Sun, Superior, CO (US); Ming Tregay, Cambridge, MA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/948,110

(22) Filed: Sep. 23, 2004

Related U.S. Application Data
(60) Provisional application No. 60/505,524, filed on Sep. 23, 2003.

(51) Int. Cl. A61K 31/7072; A61K 31/517; C07D 43/02

(52) U.S. Cl. 514/43; 544/284; 514/266.2; 536/27.1

(57) ABSTRACT

Bis-quinazoline compounds based on the compound (3,4-Dihydro-quinazolin-2-yl)-quinazolin-2-yl-amine, and methods of use of the compounds as inhibitors of bacterial DNA polymerase holoenzymes and in the treatment of bacterial infections are described.

### CROSS REFERENCE TO RELATED APPLICATIONS

This application claims the benefit under 35 U.S.C. § 119, of U.S. Provisional Patent Application Ser. No. 60/505,524, entitled “Bis-Quinazoline Compounds For The Treatment Of Drug Resistant Bacterial Infections,” filed Sep. 23, 2003, and incorporated by reference herein in its entirety.

### BACTERIAL DNA REPLICATION BACKGROUND OF THE INVENTION

Bacterial DNA replication has long been recognized as an attractive target system for new antibacterials. It is an essential process and stalled DNA replication can trigger cell death. The bacterial DNA replication complex is target-rich and involves as much as 6% of the essential proteins in bacteria, and its proper functioning is based on multiple, dynamic enzyme-substrate, protein-protein, and protein-DNA interactions. Replication proteins tend to be highly conserved among bacteria but substantially different from eukaryotic systems at the amino acid sequence level, which may facilitate the identification of compounds that selectively disrupt bacterial DNA replication. With only a few copies per cell, the replication complex is a significant point of pathogen susceptibility and even very low concentrations of an inhibitor can shut down DNA replication.

Inhibition of bacterial DNA polymerase holoenzymes will be beneficial in the treatment of bacterial infections especially against those organisms that have developed resistance to existing chemotherapeutics.

### DETAILED DESCRIPTION OF THE INVENTION

The present invention provides compounds useful in the treatment of bacterial infections, including resistant bacterial infections, as well as pharmaceutical compositions for such treatment. It has now been found that (3,4-Dihydro-quinazolin-2-yl)-quinazolin-2-yl-amine-based compounds are useful in the treatment of bacterial infections. The compounds, referred to in general as bis-quinazolines herein, are represented by formula (I): 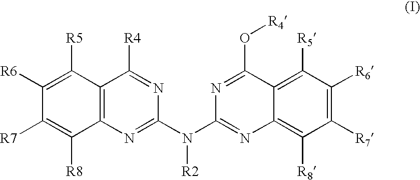
wherein
R₂, R₄, and R₄′ are independently selected from the group consisting of hydrogen, alkyl, alkenyl, alkynyl, CH₂-aryl, CH₂-heterocyclic, and phenyl; and
   wherein
R₅, R₅′, R₆, R₆′, R₇, R₇′, R₈, and R₈′ are independently selected from the group consisting of hydrogen, halo, nitro, cyano, and hydroxy, C₍₁₋₇₎alkyl (optionally substituted by halo, hydroxy, amino), mono to perfluoro C₍₁₋₃₎alkyl, C₍₃₋₇₎cycloalkyl, C₍₁₋₇₎alkoxy, amino, mono- or di-C₍₁₋₆₎alkylamino, C₍₁₋₇₎alkylthio, benzyloxy, alkenyl, O-alkenyl, S-alkenyl, NH-alkenyl, alkynyl, O-alkynyl, S-alkynyl, NH-alkynyl, CH₂-aryl, CH₂-heterocyclic, aryl, heterocyclic, S-aryl, O-aryl, NH-aryl, phenyl, O-carbohydrate, S-carbohydrate, NH-carbohydrate, sulfamoyl, carbamoyl, N-alkyl fused to aryl or heterocyclic, OCOR₉, SCOR₉, NHCOR₉, NHR₉R₁₀; wherein R₉ and R₁₀ are independently selected from the group consisting of alkyl, aryl, including heterocyclic, and acyl donors selected from the group consisting of alkyl acids, amino acids, and aromatic acids;
and a cyclic ring connecting any of R₅ and R₆; R₆ and R₇; R₇ and R₈; R₅′ and R₆′; R₆′ and R₇′; and R₇′ and R₈′, wherein the cyclic ring is selected from the group consisting of methylenedioxy, dioxane, substituted methylenedioxy, heteroatom five member ring, and heteroatom six member ring; and
   wherein
R₆, R₇, and R₆′ may also be further independently selected from the group consisting of Linker-Q,
   wherein
Linker is a C₁₋₁₀ straight chain or branched alkyl, alkenyl, or alkynyl, wherein one or more methylene groups are optionally independently replaced by O, N or S; and wherein said linker is optionally substituted with 0-2 oxo groups and one or more C₁₋₄ branched or unbranched alkyl which may be substituted by one or more halogen atoms; and
   wherein
Q is selected from the group consisting of phenyl; naphthyl; pyridine; pyrimidine; pyridazine; imidazole; benzimidazole; pyrazole; triazole; furan; thiophene; pyran; naphthyridine; oxazo[4,5-b]pyridine; and imidazo[4,5-b]pyridine; each of which may be optionally substituted with one to three groups selected from the group of halogen, C₁₋₆ alkyl, C₁₋₆ alkoxy, hydroxy, mono- or di-(C₁₋₃ alkyl)amino, C₁₋₆ alkyl-S(O)ₘ and phenylamino, wherein the phenyl ring is optionally substituted with one to two groups from the group halogen, C₁₋₆ alkyl and C₁₋₆ alkoxy;
tetrahydropyran; tetrahydrofuran; 1,3-dioxolanone; 1,3-dioxanone; 1,4-dioxane; morpholine; thiomorpholine; thiomorpholine sulfoxide; thiomorpholine sulfone; piperidine; piperazine; piperidinone; pyrrolidine; tetrahydrpyrimidone; cyclohexanone; cyclohexanol; each of which may be optionally substituted with one to three groups selected from the group consisting of C₁₋₆ alkyl, C₁₋₆ alkoxy, hydroxy, mono- or di-(C₁₋₃ alkyl)amino-C₁₋₃ alkyl, phenylamino-C₁₋₃ alkyl and C₁₋₃ alkoxy-C₁₋₃ alkyl;
C₁₋₆ alkoxy; secondary or tertiary amine wherein the amino nitrogen is covalently bonded to groups selected from the group consisting of C₁₋₆ alkyl, C₁₋₅ alkoxyalkyl, C₁₋₆ branched alkyl, each of which are optionally substituted with halo, hydroxy, or amino;
secondary or tertiary amine wherein the amino nitrogen is covalently bonded to phenyl, C₁₋₂ alkylphenyl or C₁₋₂ alkylpyridyl, wherein the aryl ring is optionally substituted with one to two groups selected from the group consisting of halogen, C₁₋₆ alkoxy, and hydroxy.

In some embodiments of the invention the compound of formula (I) is a compound of the formula (II): 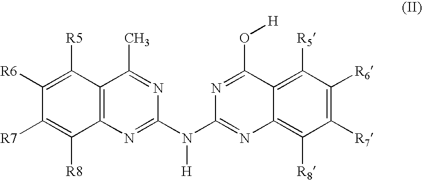
wherein
R₅, R₅′, R₆, R₆′, R₇, R₇′, R₈, and R₈′ are independently selected from the group consisting of hydrogen; halo; hydroxy; C₍₁₋₇₎alkyl (optionally substituted by halo, hydroxy, amino); mono to perfluoro C₍₁₋₃₎alkyl; C₍₃₋₇₎cycloalkyl; C₍₁₋₇₎alkoxy; amino; and mono- or di-C₍₁₋₆₎alkylamino; and a cyclic ring connecting any of R₅ and R₆; R₆ and R₇; R₇ and R₈; R₅′ and R₆′; R₆′ and R₇′; and R₇′ and R₈′, wherein the cyclic ring is selected from the group consisting of methylenedioxy, dioxane, substituted methylenedioxy, heteroatom five member ring, and heteroatom six member ring; and
   wherein
R₆, R₇, and R₆′ may also be further independently selected from the group consisting of Linker-Q,
   wherein
Linker is a C₁₋₆ straight chain or branched alkyl, alkenyl, or alkynyl, wherein one or more methylene groups are optionally independently replaced by O, N or S; and wherein said Linker is optionally substituted with 0-2 oxo groups and one or more C₁₋₄ branched or unbranched alkyl which may be substituted by one or more halogen atoms; and
Q is selected from the group consisting of pyridine, pyrazole, triazole, morpholine, piperidine, piperazine, pyrrolidine, each of which may be optionally substituted;
C₁₋₄ alkoxy; secondary or tertiary amine wherein the amino nitrogen is covalently bonded to groups selected from the group consisting of C₁₋₄ alkyl, C₁₋₅ alkoxyalkyl, and C₁₋₆ branched alkyl, each of which may be optionally substituted with halo, hydroxy, or amino;
secondary or tertiary amine wherein the amino nitrogen is covalently bonded to groups selected from phenyl, C₁₋₂ alkylphenyl and C₁₋₂ alkylpyridyl, wherein the aryl ring is optionally substituted with one to two groups selected from the group consisting of halogen, C₁₋₃ alkoxy, and hydroxyl.

Examples of Linker are methoxy, ethoxy, or propoxy each being optionally substituted.

Any compounds of this invention containing one or more asymmetric carbon atoms may occur as racemates, single enantiomers, diastereomeric mixtures and individual diastereomers. All such isomeric forms of these compounds are expressly included in the present invention. Each stereogenic carbon may be in the R or S configuration, or a combination of configurations.

When used herein, the term “alkyl” and similar terms such as “alkoxy” includes all straight-chain, cyclic-chain, and branched-chain saturated aliphatic hydrocarbon fragment containing the specified number of carbon atoms, or where no number is specified C₁-C₂₀ carbons, preferably C₁-C₇ carbons. An alkyl may be unsubstituted (i.e., H at all saturated positions) or may optionally include substitutions, such as F for H at any or all saturated positions (i.e., monofluoro substituted to perfluoro substituted). Examples of alkyl fragments include, but are not limited to, methyl, ethyl, n-propyl, isopropyl, n-butyl, isobutyl, sec-butyl, tert-butyl, pentyl, isoamyl, n-hexyl and the like. The term alkyl includes heteroalkyl groups.

The term “alkenyl” refers to any of the above alkyl groups further containing at least one carbon to carbon double bond. “Alkenyl” is intended to include hydrocarbon chains of either a straight or branched configuration and one or more unsaturated carbon-carbon bonds which may occur in any stable point along the chain, such as ethenyl, 1-propenyl, 2-propenyl, 1-butenyl, 2-butenyl, 3-butenyl, 1,3-butadienyl and the like.

The term “alkynyl” refers to any of the above alkyl groups further containing at least one carbon to carbon triple bond As used herein, the term “alkenyl” refers to a hydrocarbon radical having from two to ten carbons and at least one carbon-carbon double bond, optionally substituted with substituents selected from the group consisting of: lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. As used herein, the term “alkynyl” refers to a hydrocarbon radical having from two to ten carbons and at least one carbon-carbon triple bond, optionally substituted with substituents selected from the group consisting of: lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed.

The term “alkoxy” refers to an alkyl ether fragment, wherein the term “alkyl” is defined above. Examples of suitable alkyl ether fragments include, but are not limited to, methoxy, ethoxy, n-propoxy, isopropoxy, n-butoxy, isobutoxy, sec-butoxy, tert-butoxy and the like.

The term “aryl,” alone or in combination with any other term, refers to a carbocyclic aromatic fragment (such as phenyl or naphthyl) containing the specified number of carbon atoms, preferably from 6-15 carbon atoms, and more preferably from 6-10 carbon atoms, optionally substituted with one or more substituents selected from C₁₋₆ alkoxy, (for example methoxy), nitro, halogen, (for example chloro), amino, carboxylate and hydroxy. Examples of aryl fragments include, but are not limited to phenyl, naphthyl, indenyl, indanyl, azulenyl, fluorenyl, anthracenyl and the like.

The term “heterocyclic” alone or in combination with any other term, refers to a group having from 1 to 2 heteroatoms in a ring and the remainder of the ring atoms are carbon atoms. Suitable heteroatoms include nitrogen, oxygen, and sulfur. Suitable heterocyclic aryl groups include pyridyl, furanyl, thienyl, pyrrolyl, and the like all optionally substituted. Examples of such groups include imidazolyl, imidazolinoyl, imidazolidinyl, quinolyl, isoqinolyl, indoyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxanthiolyl, benzodioxolyl, dithiolyl, thiophenyl, tetrahydrothiophenyl, sulfolanyl, dioxanyl, dioxolanyl, tetahydrofurodihydrofuranyl, tetrahydropyranodihydrofuranyl, dihydropyranyl, tetradyrofurofuranyl and tetrahydropyranofuranyl.

As used herein, “carbohydrate” refers to a chemical moiety comprising the general composition (C)ₙ(H₂O)ₙ, including, but not limited to glucose, galactose, fucose, fructose, saccharose, mannose, arabinose, xylose, sorbose, lactose, and derivatives thereof, including but not limited to compounds which have other elemental compositions, such as aldonic acids, uronic acids, deoxysugars, or which contain additional elements or moieties, such as amino sugars wherein n is typically 4, 5, 6, 7 atoms and wherein the oxygen atom in the carbohydrate can be replaced by a heteroatom such as nitrogen, sulfur, carbon etc. A carbohydrate as used herein is understood to include chemical structures wherein “H” of any hydroxy group is replaced by any chemically compatible moiety “R”, which can be any monomer, oligomer or polymer in the meaning as used herein. Carbohydrates can be saturated or unsaturated. Carbohydrates may be charged or uncharged. Suitable charged carbohydrates include galacturonic acid, glucuronic acid, and sialic acid.

The following compounds are representative of formula (I)
6-Methoxy-4-methyl-quinazolin-2-yl)-(4-methoxy-quinazolin-2-yl)-methyl-amine;
2-[(6-Methoxy-4-methyl-quinazolin-2-yl)-methyl-amino]-quinazolin-4-ol;
2-(4-Fluoromethyl-6-methoxy-quinazolin-2-ylamino)-quinazolin-4-ol;
2-(4-Ethyl-6-methoxy-quinazolin-2-ylamino)-quinazolin-4-ol;
2-(6-Methoxy-4-propyl-quinazolin-2-ylamino)-quinazolin-4-ol;
2-(4-Fluoromethyl-6,7-dimethoxy-quinazolin-2-ylamino)-8-methoxy-quinazolin-4-ol;
2-(4-Fluoromethyl-6,7-dimethoxy-quinazolin-2-ylamino)-5-methoxy-quinazolin-4-ol.

The following compounds are representative of formula (II):
2-(4-Hydroxy-quinazolin-2-ylamino)-4-methyl-quinazolin-6-ol;
6,7,8-Trimethoxy-2-(6-methoxy-4-methyl-quinazolin-2-ylamino)-quinazolin-4-ol trifluoroacetate salt;
2-(6-Methoxy-4-methyl-quinazolin-2-ylamino)-6,8-dimethyl-quinazolin-4-ol;
2-(6-Methoxy-quinazolin-2-ylamino)-8-methyl-quinazolin-4-ol;
2-(6-Methoxy-4-methyl-quinazolin-2-ylamino)-8-methyl-6-nitro-quinazolin-4-ol;
6-Amino-2-(6-methoxy-4-methyl-quinazolin-2-ylamino)-8-methyl-quinazolin-4-ol trifluoroacetate salt;
6-N-methylamino-2-(6-methoxy-4-methyl-quinazolin-2-ylamino)-8-methyl-quinazolin-4-ol trifluoroacetate salt;
6-N-dimethylamino-2-(6-methoxy-4-methyl-quinazolin-2-ylamino)-8-methyl-quinazolin-4-ol trifluoroacetate salt;
7-Fluoro-2-(6-methoxy-4-methyl-quinazolin-2-ylamino)-6-nitro-quinazolin-4-ol trifluoroacetate salt;
2-(8-Methoxy-4-methyl-5-nitro-quinazolin-2-ylamino)-quinazolin-4-ol trifluoroacetate salt;
2-(5,8-Dimethoxy-4-methyl-quinazolin-2-ylamino)-quinazolin-4-ol trifluoroacetate salt;
2-(5,6,7-Trimethoxy-4-methyl-quinazolin-2-ylamino)-quinazolin-4-ol trifluoroacetate salt;
2-(5-Methoxy-4,8-dimethyl-quinazolin-2-ylamino)-quinazolin-4-ol trifluoroacetate salt;
2-(8-Methoxy-4-methyl-5-nitro-quinazolin-2-ylamino)-6,8-dimethyl-quinazolin-4-ol, trifluoroacetate salt;
4-Hydroxy-2-(6-methoxy-4-methyl-quinazolin-2-ylamino)-quinazoline-7-carboxylic acid;
2-(7-Fluoro-6-methoxy-4-methyl-quinazolin-2-ylamino)-quinazolin-4-ol trifluoroacetate salt;
6-Chloro-2-(6-methoxy-4-methyl-quinazolin-2-ylamino)-quinazolin-4-ol;
2-(4-Methyl-6-methylsulfanyl-quinazolin-2-ylamino)-quinazolin-4-ol trifluoroacetate salt;
6,8-Dimethyl-2-(5,6,7-trimethoxy-4-methyl-quinazolin-2-ylamino)-quinazolin-4-ol trifluoroacetate salt;
5-Methoxy-2-(6-methoxy-4-methyl-quinazolin-2-ylamino)-quinazolin-4-ol trifluoroacetate salt;
N-[4-Hydroxy-2-(6-methoxy-4-methyl-quinazolin-2-ylamino)-quinazolin-6-yl]-acetamide trifluoroacetate salt;
8-Bromo-2-(6-methoxy-4-methyl-quinazolin-2-ylamino)-6-methyl-quinazolin-4-ol trifluoroacetate salt;
2-(5-Methoxy-4,8-dimethyl-quinazolin-2-ylamino)-6,8-dimethyl-quinazolin-4-ol trifluoroacetate salt;
2-(6,7-Dimethoxy-4-methyl-quinazolin-2-ylamino)-6,8-dimethyl-quinazolin-4-ol trifluoroacetate salt;
2-(2,2-Difluoro-8-methyl-[1,3]dioxolo[4,5-g]quinazolin-6-ylamino)-quinazolin-4-ol trifluoroacetate salt;
2-(5,8-Dimethoxy-4-methyl-quinazolin-2-ylamino)-6,8-dimethyl-quinazolin-4-ol trifluoroacetate salt;
2-(7-Fluoro-6-methoxy-4-methyl-quinazolin-2-ylamino)-6,8-dimethyl-quinazolin-4-ol trifluoroacetate salt;
2-(6,8-Dimethoxy-4-methyl-quinazolin-2-ylamino)-8-methyl-quinazolin-4-ol trifluoroacetate salt;
6-Amino-2-(6-methoxy-4-methyl-quinazolin-2-ylamino)-quinazolin-4-ol; trifluoroacetate salt;
2-(8-Methoxy-4-methyl-quinazolin-2-ylamino)-6,8-dimethyl-quinazolin-4-ol;
2-(8-Methoxy-4-methyl-quinazolin-2-ylamino)-8-methyl-quinazolin-4-ol;
2-(6,7-Dimethoxy-4-methyl-quinazolin-2-ylamino)-quinazolin-4-ol;
2-(6-Methoxy-4-methyl-quinazolin-2-ylamino)-6-nitro-quinazolin-4-ol;
2-(6-Methoxy-4-methyl-quinazolin-2-ylamino)-7-nitro-quinazolin-4-ol;
5-Fluoro-2-(6-methoxy-4-methyl-quinazolin-2-ylamino)-quinazolin-4-ol;
6-Fluoro-2-(6-methoxy-4-methyl-quinazolin-2-ylamino)-quinazolin-4-ol;
7-Fluoro-2-(6-methoxy-4-methyl-quinazolin-2-ylamino)-quinazolin-4-ol;
2-(8-Methoxy-4-methyl-quinazolin-2-ylamino)-quinazolin-4-ol;
2-(8-Ethoxy-4-methyl-quinazolin-2-ylamino)-quinazolin-4-ol;
2-(6-Methoxy-4-methyl-quinazolin-2-ylamino)-quinazoline-4,6-diol;
6-Methoxy-2-(6-methoxy-4-methyl-quinazolin-2-ylamino)-quinazolin-4-ol;
6,7-Dimethoxy-2-(6-methoxy-4-methyl-quinazolin-2-ylamino)-quinazolin-4-ol;
2-(6-Methoxy-4-methyl-quinazolin-2-ylamino)-6-methyl-quinazolin-4-ol;
2-(6-Methoxy-4-methyl-quinazolin-2-ylamino)-7-methyl-quinazolin-4-ol;
2-(6-Methoxy-4-methyl-quinazolin-2-ylamino)-8-methyl-quinazolin-4-ol;
2-(6,7-Dimethoxy-4-methyl-quinazolin-2-ylamino)-8-methyl-6-nitro-quinazolin-4-ol trifluoroacetate salt;
2-(6,8-Dimethoxy-4-methyl-quinazolin-2-ylamino)-8-methyl-6-nitro-quinazolin-4-ol trifluoroacetate salt;
2-(8-Methoxy-4-methyl-quinazolin-2-ylamino)-8-methyl-6-nitro-quinazolin-4-ol trifluoroacetate salt;
2-[6-(2-Methoxy-ethoxy)-4-methyl-quinazolin-2-ylamino]-8-methyl-6-nitro-quinazolin-4-ol trifluoroacetate salt;
6-Amino-2-[6-(2-methoxy)-4-methyl-quinazolin-2-ylamino]-8-methyl-quinazolin-4-ol trifluoroacetate salt;
2-(6-Methoxy-4-methyl-quinazolin-2-ylamino)-quinazoline-4,8-diol trifluoroacetate salt;
2-(8-Methoxy-4-methyl-quinazolin-2-ylamino)-quinazoline-4,8-diol trifluoroacetate salt;
2-(7,8-Dimethoxy-4-methyl-quinazolin-2-ylamino)-6,8-dimethyl-quinazolin-4-ol trifluoroacetate salt;
2-(7,8-Dimethoxy-4-methyl-quinazolin-2-ylamino)-quinazolin-4-ol trifluoroacetate salt;
2-(6,8-Dimethoxy-4-methyl-quinazolin-2-ylamino)-6,8-dimethyl-quinazolin-4-ol trifluoroacetate salt;
2-(6-methoxy-7-amino-4-methyl-quinazolin-2-ylamino)-8-methoxy-quinazolin-4-ol trifluoroacetate salt;
2-(6-methoxy-7-nitro-4-methyl-quinazolin-2-ylamino)-8-methoxy-quinazolin-4-ol trifluoroacetate salt;
2-(7,8-Dimethoxy-4-methyl-quinazolin-2-ylamino)-8-methoxy-quinazolin-4-ol trifluoroacetate salt;
8-Methoxy-2-[6-methoxy-8-(2-methoxy-ethoxy)-4-methyl-quinazolin-2-ylamino]-quinazolin-4-ol trifluoroacetate salt;
8-Methoxy-2-[6-(2-methoxy-ethoxy)-4-methyl-quinazolin-2-ylamino]-quinazolin-4-ol trifluoroacetate salt;
8-Methoxy-2-[7-methoxy-8-(2-methoxy-ethoxy)-4-methyl-quinazolin-2-ylamino]-quinazolin-4-ol trifluoroacetate salt;
8-Methoxy-2-(8-methyl-2,3-dihydro-1,4-dioxa-5,7-diaza-phenanthren-6-ylamino)-quinazolin-4-ol;
5-ethoxy-2-(8-methyl-2,3-dihydro-1,4-dioxa-5,7-diaza-phenanthren-6-ylamino)-quinazolin-4-ol trifluoroacetate salt;
2-(6,7-Dimethoxy-4-methyl-quinazolin-2-ylamino)-5-methoxy-quinazolin-4-ol trifluoroacetate salt;
2-(6,8-Dimethoxy-4-methyl-quinazolin-2-ylamino)-5-methoxy-quinazolin-4-ol trifluoroacetate salt;
2-(7-Fluoro-6-methoxy-4-methyl-quinazolin-2-ylamino)-8-methoxy-quinazolin-4-ol trifluoroacetate salt;
5-Methoxy-2-[6-(2-methoxy-ethoxy)-4-methyl-quinazolin-2-ylamino]-quinazolin-4-ol trifluoroacetate salt;
2-[6,7-Bis-(2-methoxy-ethoxy)-4-methyl-quinazolin-2-ylamino]-8-methoxy-quinazolin-4-ol trifluoroacetate salt;
8-Methoxy-2-(5-methoxy-4,8-dimethyl-quinazolin-2-ylamino)-quinazolin-4-ol trifluoroacetate salt;
8-Methyl-2-(5-methoxy-4,8-dimethyl-quinazolin-2-ylamino)-quinazolin-4-ol trifluoroacetate salt;
2-(6,8-Dimethoxy-4-methyl-quinazolin-2-ylamino)-quinazoline-4,5-diol trifluoroacetate salt;
2-(7-Fluoro-6-methoxy-4-methyl-quinazolin-2-ylamino)-5-methoxy-quinazolin-4-ol trifluoroacetate salt;
5,8-Dimethoxy-2-(8-methyl-2,3-dihydro-1,4-dioxa-5,7-diaza-phenanthren-6-ylamino)-quinazolin-4-ol trifluoroacetate salt;
2-(6,7-Dimethoxy-4-methyl-quinazolin-2-ylamino)-8-fluoro-quinazolin-4-ol trifluoroacetate salt;
2-(6,7-Dimethoxy-4-methyl-quinazolin-2-ylamino)-8-ethoxy-quinazolin-4-ol trifluoroacetate salt;
2-(6,8-Dimethoxy-4-methyl-quinazolin-2-ylamino)-8-fluoro-quinazolin-4-ol trifluoroacetate salt;
2-(6,7-Dimethoxy-4-methyl-quinazolin-2-ylamino)-5,8-dimethoxy-quinazolin-4-ol trifluoroacetate salt;
2-(6,8-Dimethoxy-4-methyl-quinazolin-2-ylamino)-5,8-dimethoxy-quinazolin-4-ol trifluoroacetate salt;
[2-(6,7-Dimethoxy-4-methyl-quinazolin-2-ylamino)-4-hydroxy-quinazolin-6-yloxy]acetonitrile;
2-(6,7-Dimethoxy-4-methyl-quinazolin-2-ylamino)-6-(2-methoxy-ethoxy)-quinazolin-4-ol trifluoroacetate salt;
2-(4-Hydroxy-8-methoxy-quinazolin-2-ylamino)-6-methoxy-4-methyl-quinazolin-7-ol trifluoroacetate salt;
2-(8-Fluoro-4-hydroxy-quinazolin-2-ylamino)-6-methoxy-4-methyl-quinazolin-7-ol trifluoroacetate salt;
6-(2-Chloro-ethoxy)-2-(6,7-dimethoxy-4-methyl-quinazolin-2-ylamino)-quinazolin-4-ol trifluoroacetate salt;
6-(3-Chloro-propoxy)-2-(6,7-dimethoxy-4-methyl-quinazolin-2-ylamino)-quinazolin-4-ol trifluoroacetate salt;
6-(2-Chloro-ethoxy)-2-(6,8-dimethoxy-4-methyl-quinazolin-2-ylamino)-quinazolin-4-ol trifluoroacetate salt;
8-Fluoro-2-(4-methyl-7,8-dihydro-[1,4]dioxino[2,3-g]quinazolin-2-ylamino)-quinazolin-4-trifluoroacetate salt;
7-(6,7-Dimethoxy-4-methyl-quinazolin-2-ylamino)-1,3-dioxa-6,8-diaza-cyclopenta[a]naphthalen-9-ol trifluoroacetate salt;
2-(6,7-Dimethoxy-4-methyl-quinazolin-2-ylamino)-5-isopropoxy-quinazolin-4-ol trifluoroacetate salt;
2-(6,8-Dimethoxy-4-methyl-quinazolin-2-ylamino)-5-isopropoxy-quinazolin-4-ol trifluoroacetate salt;
2-(6,8-Dimethoxy-4-methyl-quinazolin-2-ylamino)-5-ethoxy-quinazolin-4-ol trifluoroacetate salt;
2-(6,7-Dimethoxy-4-methyl-quinazolin-2-ylamino)-6-piperidin-1-yl-quinazolin-4-ol trifluoroacetate salt;
2-(6,7-Dimethoxy-4-methyl-quinazolin-2-ylamino)-6-morpholin-4-yl-quinazolin-4-ol trifluoroacetate salt;
2-[6-(2-Diethylamino-ethoxy)-4-methyl-quinazolin-2-ylamino]-8-methoxy-quinazolin-4-ol trifluoroacetate salt;
8-Methoxy-2-(6,7,8-trimethoxy-4-methyl-quinazolin-2-ylamino)-quinazolin-4-ol trifluoroacetate salt;
2-(7-Fluoro-8-methoxy-4-methyl-quinazolin-2-ylamino)-8-methoxy-quinazolin-4-ol trifluoroacetate salt;
8-Fluoro-2-(6,7,8-trimethoxy-4-methyl-quinazolin-2-ylamino)-quinazolin-4-ol trifluoroacetate salt;
8-Fluoro-2-(7-fluoro-8-methoxy-4-methyl-quinazolin-2-ylamino)-quinazolin-4-ol trifluoroacetate salt;
6-Amino-2-(6,7-dimethoxy-4-methyl-quinazolin-2-ylamino)-8-methyl-quinazolin-4-ol trifluoroacetate salt;
2-(6,7-Dimethoxy-4-methyl-quinazolin-2-ylamino)-6-dimethylamino-8-methyl-quinazolin-4-ol trifluoroacetate salt;
8-Amino-2-(6-methoxy-4-methyl-quinazolin-2-ylamino)-quinazolin-4-ol trifluoroacetate salt;
6-Amino-2-(6-methoxy-4-methyl-quinazolin-2-ylamino)-8-methoxy-quinazolin-4-ol trifluoroacetate salt;
6-Amino-2-(6,8-dimethoxy-4-methyl-quinazolin-2-ylamino)-8-methoxy-quinazolin-4-ol trifluoroacetate salt;
6-Amino-2-[6-(2-diethylamino-ethoxy)-4-methyl-quinazolin-2-ylamino]-8-methyl-quinazolin-4-ol trifluoroacetate salt;
6-Amino-2-(6,8-dimethoxy-4-methyl-quinazolin-2-ylamino)-8-methyl-quinazolin-4-ol trifluoroacetate salt;
2-(7-Dimethylamino-6-methoxy-4-methyl-quinazolin-2-ylamino)-8-methoxy-quinazolin-4-ol trifluoroacetate salt;
2-(6,7-Dimethoxy-4-methyl-quinazolin-2-ylamino)-5-dimethylamino-quinazolin-4-ol trifluoroacetate salt;
2-(6,7-Dimethoxy-4-methyl-quinazolin-2-ylamino)-6-(2H-tetrazol-5-ylmethoxy)-quinazolin-4-ol trifluoroacetate salt;
7-(6,8-Dimethoxy-4-methyl-quinazolin-2-ylamino)-1,3-dioxa-6,8-diaza-cyclopenta[a]naphthalen-9-ol trifluoroacetate salt;
7-(6,8-Dimethoxy-4-methyl-quinazolin-2-ylamino)-1,3-dioxa-6,8-diaza-cyclopenta[a]naphthalen-9-ol HCl salt;
2-(6,7-Dimethoxy-4-methyl-quinazolin-2-ylamino)-5,6-dimethoxy-quinazolin-4-ol TFA salt;
2-(6,7-Dimethoxy-4-methyl-quinazolin-2-ylamino)-5,6-dimethoxy-quinazolin-4-ol HCl salt;
2-(6,8-Dimethoxy-4-methyl-quinazolin-2-ylamino)-5,6-dimethoxy-quinazolin-4-ol TFA salt;
2-(6,8-Dimethoxy-4-methyl-quinazolin-2-ylamino)-5,6-dimethoxy-quinazolin-4-ol HCl salt;
2-(6,7-Dimethoxy-4-methyl-quinazolin-2-ylamino)-6-(2-hydroxy-ethoxy)-quinazolin-4-ol trifluoroacetate salt;
2-(6,8-Dimethoxy-4-methyl-quinazolin-2-ylamino)-6-(2-hydroxy-ethoxy)-quinazolin-4-ol trifluoroacetate salt;
6-[2-(3-Hydroxy-pyrrolidin-1-yl)-ethoxy]-2-(8-methoxy-4-methyl-quinazolin-2-ylamino quinazolin-4-ol trifluoroacetate salt;
2-(6,7-Dimethoxy-4-methyl-quinazolin-2-ylamino)-6-{2-[(2-hydroxy-ethyl)-methyl-amino]-ethoxy}-quinazolin-4-ol trifluoroacetate salt;
2-(6,8-Dimethoxy-4-methyl-quinazolin-2-ylamino)-6-{2-[(2-hydroxy-ethyl)-methyl-amino]-ethoxy}-quinazolin-4-ol trifluoroacetate salt;
2-(6,8-Dimethoxy-4-methyl-quinazolin-2-ylamino)-6-(2-morpholin-4-yl-ethoxy)-quinazolin-4-ol trifluoroacetate salt;
2-(6,7-Dimethoxy-4-methyl-quinazolin-2-ylamino)-6-[2-(1-hydroxymethyl-2-methyl-propylamino)-ethoxy]-quinazolin-4-ol Trifluoroacetate salt;
2-(6,8-Dimethoxy-4-methyl-quinazolin-2-ylamino)-6-[2-(2-hydroxy-1-methyl-ethylamino)-ethoxy]-quinazolin-4-ol trifluoroacetate salt;
6-(2-Diethylamino-ethoxy)-2-(6,8-dimethoxy-4-methyl-quinazolin-2-ylamino)-quinazolin-4-ol trifluoroacetate salt;
2-(6,7-Dimethoxy-4-methyl-quinazolin-2-ylamino)-6-(2-piperidin-1-yl-ethoxy)-quinazolin-4-ol trifluoroacetate salt;
2-(6,7-Dimethoxy-4-methyl-quinazolin-2-ylamino)-6-(2-dimethylamino-ethoxy)-quinazolin-4-ol trifluoroacetate salt;
2-(6,7-Dimethoxy-4-methyl-quinazolin-2-ylamino)-6-[2-(4-methyl-piperazin-1-yl)-ethoxy]-quinazolin-4-ol trifluoroacetate salt;
2-(6,7-Dimethoxy-4-methyl-quinazolin-2-ylamino)-6-(2-pyrrolidin-1-yl-ethoxy)-quinazolin-4-ol trifluoroacetate salt;
6-(2-Diethylamino-ethoxy)-2-(6,7-dimethoxy-4-methyl-quinazolin-2-ylamino)-quinazolin-4-ol trifluoroacetate salt;
2-[4-Hydroxy-6-(2-morpholin-4-yl-ethoxy)-quinazolin-2-ylamino]-6,7-dimethoxy-quinazolin-4-ol trifluoroacetate salt;
2-(6,7-Dimethoxy-4-methyl-quinazolin-2-ylamino)-6-(3-pyrrolidin-1-yl-propoxy)-quinazolin-4-ol trifluoroacetate salt;
2-(6,7-Dimethoxy-4-methyl-quinazolin-2-ylamino)-6-(2-pyrrol-1-yl-ethoxy)-quinazolin-4-ol trifluoroacetate salt;
2-(6,7-Dimethoxy-4-methyl-quinazolin-2-ylamino)-6-(2-pyrrolidin-1-yl-ethoxy)-quinazolin-4-ol trifluoroacetate salt;
2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(3-hydroxy-piperidin-1-yl)-ethoxy]-quinazolin-4-ol trifluoroacetate salt;
6-{2-[Bis-(2-hydroxy-ethyl)-amino]-ethoxy}-2-(6,7-dimethoxy-4-methyl-quinazolin-2-ylamino)-quinazolin-4-ol trifluoroacetate salt;
2-(6,7-Dimethoxy-4-methyl-quinazolin-2-ylamino)-6-{2-[ethyl-(2-hydroxy-ethyl)-amino]-ethoxy}-quinazolin-4-ol trifluoroacetate salt;
2-(6,8-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(2-hydroxy-ethylamino)-ethoxy]-quinazolin-4-ol trifluoroacetate salt;
2-(6,7-Dimethoxy-4-methyl-quinazolin-2-ylamino)-6-{2-[(1-hydroxymethyl-3-methyl butyl)-amino]-ethoxy}-quinazolin-4-ol Trifluoroacetate salt;
6-[2-(3-Amino-pyrrolidin-1-yl)-ethoxy]-2-(6,7-dimethoxy-4-methyl-quinazolin-2-ylamino)-quinazolin-4-ol;
2-(6,7-Dimethoxy-4-methyl-quinazolin-2-ylamino)-6-[2-(2-hydroxymethyl-pyrrolidin-1-yl)-ethoxy]-quinazolin-4-ol trifluoroacetate salt;
2-{2-[2-(6,7-Dimethoxy-4-methyl-quinazolin-2-ylamino)-4-hydroxy-quinazolin-6-yloxy]-ethylamino}-propane-1,3-diol trifluoroacetate salt;
2-(6,8-Dimethoxy-4-methyl-quinazolin-2-ylamino)-6-(2-pyrrolidin-1-yl-ethoxy)-quinazolin-4-ol trifluoroacetate salt;
2-(6,8-Dimethoxy-4-methyl-quinazolin-2-ylamino)-6-{2-[ethyl-(2-hydroxy-ethyl)-amino]-ethoxy}-quinazolin-4-ol trifluoroacetate salt;
2-(6,8-Dimethoxy-4-methyl-quinazolin-2-ylamino)-6-[2-(2-hydroxymethyl-pyrrolidin-1-yl)-ethoxy]-quinazolin-4-ol trifluoroacetate salt;
2-(6,7-Dimethoxy-4-methyl-quinazolin-2-ylamino)-6-[2-(2-hydroxy-1-methyl-ethylamino)-ethoxy]-quinazolin-4-ol trifluoroacetate salt;
2-(6,7-Dimethoxy-4-methyl-quinazolin-2-ylamino)-6-[2-(2-hydroxymethyl-morpholin-4-yl)-ethoxy]-quinazolin-4-ol trifluoroacetate salt;
2-(6,7-Dimethoxy-4-methyl-quinazolin-2-ylamino)-6-{2-[(2-dimethylamino-ethyl)-ethyl-amino]-ethoxy}-quinazolin-4-ol trifluoroacetate salt;
2-(6,7-Dimethoxy-4-methyl-quinazolin-2-ylamino)-6-[2-(morpholin-4-ylamino)-ethoxy]-quinazolin-4-ol trifluoroacetate salt;
2-(6,7-Dimethoxy-4-methyl-quinazolin-2-ylamino)-6-[2-(2-hydroxymethyl-piperidin-1-yl)-ethoxy]-quinazolin-4-ol trifluoroacetate salt;
2-(6,7-Dimethoxy-4-methyl-quinazolin-2-ylamino)-6-(2-[1,2,4]triazol-4-yl-ethoxy)-quinazolin-4-ol trifluoroacetate salt;
2-(6,7-Dimethoxy-4-methyl-quinazolin-2-ylamino)-6-{2-[4-(2-hydroxy-ethyl)-piperazin-1-yl]-ethoxy}-quinazolin-4-ol trifluoroacetate salt;
2-(6,7-Dimethoxy-4-methyl-quinazolin-2-ylamino)-6-{2-[N′-(2-hydroxy-ethyl)-hydrazino]-ethoxy}-quinazolin-4-ol trifluoroacetate salt;
2-(6,7-Dimethoxy-4-methyl-quinazolin-2-ylamino)-6-[2-(2,6-dimethyl-morpholin-4-yl)-ethoxy]-quinazolin-4-ol trifluoroacetate salt;
2-(6,7-Dimethoxy-4-methyl-quinazolin-2-ylamino)-6-{N′,N′-dimethyl-hydrazino]-ethoxy}-quinazolin-4-ol trifluoroacetate salt;
2-(6,7-Dimethoxy-4-methyl-quinazolin-2-ylamino)-6-{2-[2-(2-hydroxy-ethoxy)-ethylamino]-ethoxy}-quinazolin-4-ol trifluoroacetate salt;
2-(6,7-Dimethoxy-4-methyl-quinazolin-2-ylamino)-6-[2-(2-pyrrolidin-1-ylmethyl-pyrrolidin-1-yl)-ethoxy]-quinazolin-4-ol trifluoroacetate salt;
2-(6,7-Dimethoxy-4-methyl-quinazolin-2-ylamino)-6-[2-(2-hydroxy-cyclohexylamino)-ethoxy]-quinazolin-4-ol trifluoroacetate salt; and
2-(6,7-Dimethoxy-4-methyl-quinazolin-2-ylamino)-6-[2-(methyl-pyridin-3-ylmethyl-amino)-ethoxy]-quinazolin-4-ol trifluoroacetate salt;
2-(6,7-Dimethoxy-4-methyl-quinazolin-2-ylamino)-6-[2-(2-pyridin-4-yl-ethylamino)-ethoxy]-quinazolin-4-ol TFA salt; and
2-(6,7-Dimethoxy-4-methyl-quinazolin-2-ylamino)-6-[2-(methyl-pyridin-4-ylmethyl-amino)-ethoxy]-quinazolin-4-ol TFA salt.

The compounds of the invention may be prepared by Method A, B or C as described in Example 1, Example 2, and Example 3, respectively.

Method A, described in Example 1, provides a method of preparing a bis-quinazoline compound of the formula: 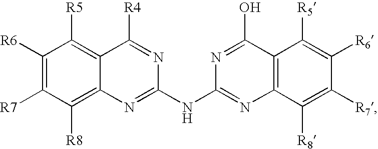
wherein R₄, R₅, R₅′, R₆, R₆′, R₇, R₇′, R₈, and R₈′ are defined as above in Formula (I). The method comprises providing a salt of an aniline of the formula 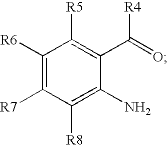
reacting it with sodium dicyanamide to generate a quinazolin-2-yl-cyanamide of the formula: 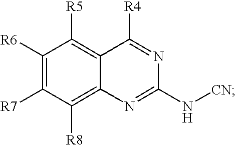
and reacting the quinazolin-2-yl-cyanamide with an alkyl anthranilate of the formula: 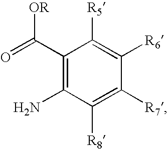
in the presence of a concentrated acid such as hydrochloric acid, sulfuric acid, or phosphoric acid, to generate a the bis-quinazoline compound.

Method B, described in Example 2, provides a method of preparing a bis-quinazoline compound of the formula: 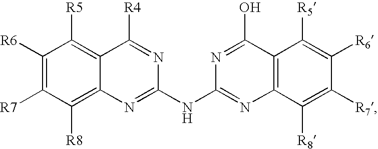
wherein R₄, R₅, R₅′, R₆, R₆′, R₇, R₇′, R₈, and R₈′ are defined as above in Formula (I). The method comprises providing a quinazolin-2-yl guanidine of the formula: 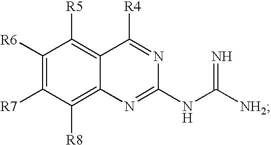
and reacting the quinazolin-2-yl guanidine with an isatoic anhydride of the formula: 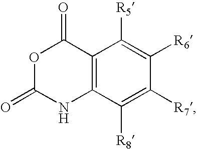
in the presence of a tertiary amine such as diiopropyethylamine and triethylamine to generate the bis-quinazoline compound.

Method C, described Example 3, provides a method of preparing a bis-quinazoline compound of the formula: 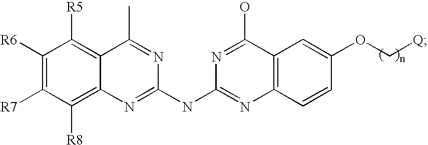
wherein
n is an integer from 1-10; and
   wherein
R₅, R₆, R₇, R₈, and Q are defined as above in Formula (I). The method comprises: providing a bis-quinazolin-2-yl-amine of the formula: 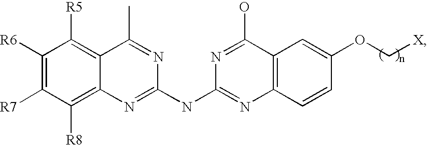
   wherein X is selected from the group consisting of Cl, Br, and I; reacting the bis-quinazolin-2-yl-amine with a nucleophile, and heating the resulting mixture to generate a bis-quinazoline compound. The nulcleophile may be a compound such as a primary amine, a secondary amine, a primary alcohol, a secondary alcohol, a primary alkoxide, a secondary alkoxide, and alkali metal, and an alkali metal hydroxide.

Quinazolin-2-yl guanidines of formula 5, as utilized in Method B, can be prepared by method D, as described in Example 4, or E as described in Example 5.

The bis-quinazoline compounds of the present have potent, reversible biochemical activity against DNA replication complexes from both Gram-positive and Gram-negative bacteria, with IC₅₀ values ranging from <50 nM to 1 μM across the series. The enzyme target within the DNA replication complex and kinetic mechanism of inhibition has been determined for the series (Ki values<1 μM, for selected compounds). These compounds show selectivity for bacterial replication targets (as compared to the eukaryotic equivalents).

Representative bis-quniazolines were tested in reconstituted bacterial DNA polymerase holoenzymes from E. coli, Y. pestis and S. pyogenes which exhibited reversible biochemical activity against DNA replication complexes from both Gram-positive and Gram-negative bacteria, with IC₅₀ values ranging from <50 nM to 1 μM across the series. Reconstituted bacterial DNA polymerase holoenzymes from A. aeolicus, T. thermophilus, T. maratima, E. coli, Y. pestis and S. pyogenes and biochemical assays are described in detail in International Patent Application Publication WO 02/06532, entitled Novel DNA Polymerase III Holoenzyme Delta Subunit Nucleic Acid Molecules And Proteins, International Patent Application Publication WO 02/092769, entitled System For Discovery Of Agents That Block Yersinia Pestis And Pseudomonas Aeruginosa DNA Replication; and International Patent Application Publication WO 02/34936, entitled Novel S. Pyogenes DNA Polymerase III Holoenzyme Nucleic Acid Molecules and Proteins. The disclosure of each of these publications is incorporated by reference herein in its entirely.

Example 183 describes such an assay.

Briefly, compounds of the present invention were tested for antibacterial activity against a wide range of gram-positive and gram-negative bacterial pathogens including strains of S. aureus (oxacillin-susceptible and -resistant), E. faecalis (vancomycin-susceptible and -resistant), E. faecium, S. pyogenes, S. pneumoniae, H. influenzae and M. catarrhalis. All compounds were tested using the broth microdilution MIC method in accordance with NCCLS guidelines. The compounds described in Examples 9, 10, 11, 12, 13, 15, 16, 18, 19, 20, 22, 23, 24, 26, 27, 30, 32, 35, 36, 37, 39, 41, 43, 44, 45, 46, 47, 48, 49, 51, 52, 58, 59, 60, 61, 62, 63, 65, 66, 67, 68, 69, 70, 71, 72, 73, 74, 75, 76, 77, 78, 79, 80, 81, 82, 83, 84, 85, 86, 87, 88, 89, 90, 91, 94, 95, 96, 97, 98, 99, 100, 102, 103, 104, 105, 107, 108, 109, 111, 112, 114, 115, 116, 117, 118, 119, 120, 121, 123, 124, 125, 126, 127, 128, 130, 131, 134, 135, 136, 137, 138, 139, 140, 141, 142, 143, 144, 145, 146, 147, 148, 149, 150, 151, 153, 154, 155, 156, 157, 158, 159, 160, 161, 162, 163, 164, 165, 166, 167, 168, 169, 170, 171, 173, 174, 175, 176, 177, 179, 180, 181, 182, 183, and 184 had MICs ranging from <0.06 to 16 μg/mL against some strains of S. aureus, E. faecalis, S. pyogenes and S. pneumoniae. The compounds described in Examples 12, 18, 19, 39, 66, 77, 84, 85, 86, 87, 88, 89, 90, 91, 94, 95, 96, 97, 98, 99, 102, 104, 108, 115, 120, 121, 125, 138, 140, 142, 143, 144, 145, 146, 147, 148, 149, 150, 151, 153, 154, 155, 156, 157, 158, 159, 160, 161, 162, 163, 164, 165, 166, 169, 170, and 171 also had activity against some strains of gram-negative bacteria including M. catarrhalis (MIC range, 2-64 μg/mL) H. influenzae (MIC range, 16-32 μg/mL) and efflux mutants of E. coli (MICs, 16-64 μg/mL) and P. aeruginosa (MICs, 64 μg/mL).

The recent findings leading to this disclosure include evidence of in vivo antibacterial activity in a murine sepsis model. As explained in detail in the Examples sections, 2-(6-Methoxy-4-methyl-quinazolin-2-ylamino)-3H-quinazolin-4-one has demonstrated biochemical activity against the reconstituted DNA polymerase holoenzymes from E. coli, Y. pestis and S. pyogenes. The compound has potent whole cell antibacterial and bactericidal activity against clinically important gram-positive pathogens and its activity is evident when tested in the presence of 50% human serum. This compound is well tolerated in mice following single dose IP administration at concentrations up to 25 mg/kg and was effective in protecting 50% of mice from a S. aureus IP infection when treated with 25 mg/kg one hour post infection. Additional bis-quinazoline compounds described herein also demonstrated 50% protection of the test subjects from a lethal infection at 25 mg/kg efficacy in the murine intra-abdominal sepsis model against S. aureus.

The murine intra-abdominal sepsis model has been widely used in early stage antibacterial discovery strategies to demonstrate the in vivo effects of novel antibacterial compounds. Typically, the model involves inoculating the intra-peritoneal cavity of the mouse with the minimum lethal dose (MLD) of the test organism. The test antimicrobial agent can be administered by any route (e.g. intravenously, sub-cutaneously, oral, intraperitoneal). Early stage compounds are evaluated following intraperitoneal administration since this is considered to be relevant to systemic treatment since most of the compound will be absorbed into the bloodstream. The compound is then monitored for its ability to protect the mouse from a lethal infection with the test organism in a concentration-dependent manner. At the same time the model provides early data on the potential toxicity of a new compound.

One aspect of the invention is therefore the use of bis-quinazolines of the present invention in the form of their base or salts of physiologically acceptable acids for the production of a therapeutic or pharmaceutical composition for the treatment of bacterial infections. The phrase “pharmaceutically acceptable salt(s)”, as used herein, unless otherwise indicated, includes salts of acidic or basic groups which may be present in the compounds of Formula I & II. The compounds of formula I & II that are basic in nature are capable of forming a wide variety of salts with various inorganic and organic acids. The acids that may be used to prepare pharmaceutically acceptable acid addition salts of such basic compounds of formula I & II are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, trifluoroacetate, and valerate salts.

The therapeutic compositions of the present invention have antibacterial activity against clinically important gram-positive pathogens including the staphylococci and streptococci and particularly including isolates resistant to currently marketed agents.

Compositions of the present invention can also include other components such as a pharmaceutically acceptable excipient, an adjuvant, and/or a carrier. For example, compositions of the present invention can be formulated in an excipient that the animal to be treated can tolerate.

Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an adjuvant or a carrier. Adjuvants are typically substances that generally enhance the immune response of an animal to a specific antigen. Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

In general, formulations which may be used for the active ingredient are conventional formulations well known in the art, for example as described in standard textbooks of pharmaceutics such as the United States Pharmacopoeia (USP), British Pharmacopoeia, European Pharmacopoeia, Japanese Pharmacopoeia, and International Pharmacopoeia.

One embodiment of the present invention is a controlled release formulation that is capable of slowly releasing a composition of the present invention into an animal. As used herein a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

The present invention is also directed toward methods of treatment utilizing the therapeutic compositions of the present invention. The method comprises administering the therapeutic agent to a subject in need of such administration.

Generally, the therapeutic bis-quinazolines used in the invention are administered to a human or animal in an effective amount. Generally, an effective amount is an amount effective to either (1) reduce the symptoms of the disease sought to be treated or (2) induce a pharmacological change relevant to treating the disease sought to be treated. For bacterial infections, an effective amount includes an amount effective to: reduce or eliminate the bacterial population; slow the spread of infection; or increase the life expectancy of the affected human or animal. A therapeutically effective dose may vary depending upon the route of administration and dosage form.

Therapeutically effective amounts of the therapeutic agents can be any amount or doses sufficient to bring about the desired effect and depend, in part, on the condition, type and location of the infection, the size, age, gender and condition of the patient, as well as other factors readily known to those skilled in the art. The dosages can be given as a single dose, or as several doses, for example, divided over the course of several weeks. Any of the above dosage forms containing effective amounts are well within the bounds of routine experimentation and therefore, well within the scope of the instant invention.

In order to protect an animal from bacterial infection, a therapeutic composition of the present invention is administered to the animal in an effective manner such that bacterial infection of animals treated with the composition is reduced. As such, a treated animal is an animal that is competent to reduce the bacterial burden. Preferably, the bacterial infection is reduced by at least about 50 percent, more preferably by at least about 70 percent and even more preferably by at least about 90 percent. Methods to administer compositions to the animal in order to render the animal competent depend on the nature of the composition and administration regime.

In a preferred embodiment, a composition of the present invention when administered to a host animal is able to reduce bacterial infection by at least about 50 percent within at least about 24 hours after administration. A more preferred composition when administered to a host animal is able to reduce bacterial infection by at least about 65 percent within at least about 24 hours after administration. An even more preferred composition when administered to an animal is able to reduce bacterial infection by at least about 90 percent within at least about 24 hours after administration.

Acceptable protocols to administer compositions in an effective manner include individual dose size, number of doses, frequency of dose administration, and mode of administration. Determination of such protocols can be accomplished by those skilled in the art. A suitable single dose is a dose that is capable of reducing bacterial infection when administered one or more times over a suitable time period. For example, a preferred single dose of compound of formula 1 ranges from about 1 microgram (μg) to about 10 milligrams (mg) of the composition per kilogram body weight of the animal. Modes of administration can include, but are not limited to, oral, nasal, topical, transdermal, rectal, and parenteral routes. Parenteral routes can include, but are not limited to subcutaneous, intradermal, intravenous, and intramuscular routes.

Compositions of the present invention can be administered to any animal susceptible to bacterial infection, such as mammals, including humans, and birds, with cats, dogs, cattle, chinchillas, ferrets, goats, mice, minks, rabbits, raccoons, rats, sheep, squirrels, swine, chickens, ostriches, quail and turkeys as well as other furry animals, pets and/or economic food animals.

### EXAMPLES

The following examples are provided for illustrative purposes only and are not intended to limit the scope of the invention.

### Example 1

### General Method A for the Preparation of Bis-Quinazolines

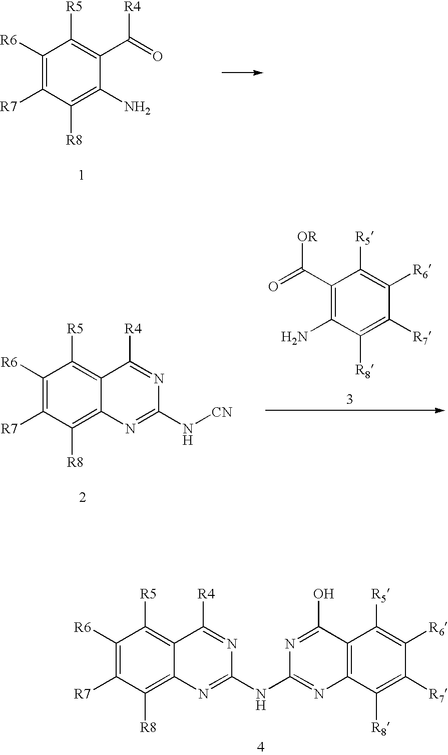

The hydrochloride salt of aniline of formula 1 is suspended in anhydrous solvent such as tetrahydrofuran and sodium dicyanamide is added. The mixture is stirred at between 20-70° C., preferred at 40° C. overnight, and subsequently filtered and washed with a solvent such as THF. The product 2 is dried under vacuum. The obtained product is suspended in an anhydrous solvent such as THF and alkyl anthranilate of formula 3 and concentrated acid such as hydrochloric. The mixture is stirred at between 20-80° C., preferably 60° C. overnight under nitrogen flow. The solid material obtained is purified by silica gel chromatography using combination of solvents such as dichloromethane, methanol and triethylamine, preferably dichloromethane, methanol and triethylamine.

### Example 2

### General Method B for the Preparation of Bis-Quinazolines

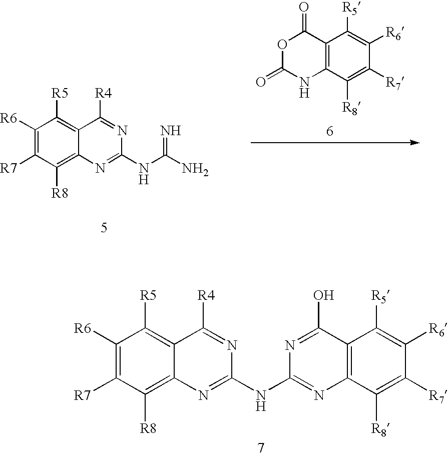

In method B, quinazolin-2-yl guanidine of formula 5 is dissolved in anhydrous solvents such as dimethylformamide, isatoic anhydride of formula 6 and tertiary amine such as diisopropylethylamine are added. The mixture is stirred at between 20-130° C., preferably at 100° C. overnight. The reaction is cooled to 0-30° C., preferably to room temperature and the product 7 is filtered and washed with a small amount of solvents such as dimethylformamide, tetrahydrofuran, and diethyl ether and dried.

### Example 3

### General Method C for the Preparation of Bis-Quinazolines

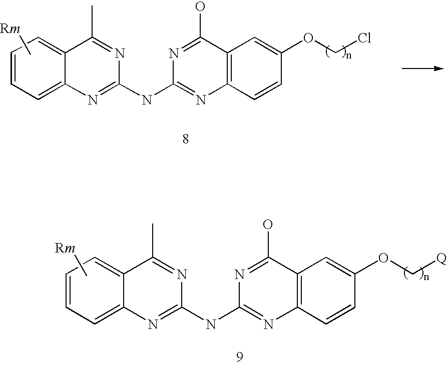

Into a sealable microwave vessel is placed a Teflon® stirbar, Bis-quinazolin-2-yl-amines of formula 8 and anhydrous solvents such as NMP. To this stirring suspension is added nucleophile such as amine, metal hydroxide or metal alkoxide. The vessel is sealed and heated at between 100-200° C., 300 W, for 1-120 minutes, preferably 150° C. for 30 minutes in the Emrys™ Optimizer, microwave synthesizer, by Personal Chemistry®. The resultant solution is precipitated into solvents such as diethyl ether. The solid material obtained is purified by silica gel chromatography using combination of solvents such as dichrolomethane, methanol with ammonia or tertiary amine such as triethylamine, preferably triethyl amine. The product is converted to a suitable pharmaceutically acceptable salt form by mixing the product solution in organic solvents with acid such as trifluoroacetic acid or hydrochloric acid and evaporating the volatile to dryness to afford Bis-quinazolin-2-yl-amines of formula 9.

### Example 4

### General Method D for the Preparation of Quinazolin-2-yl Guanidines

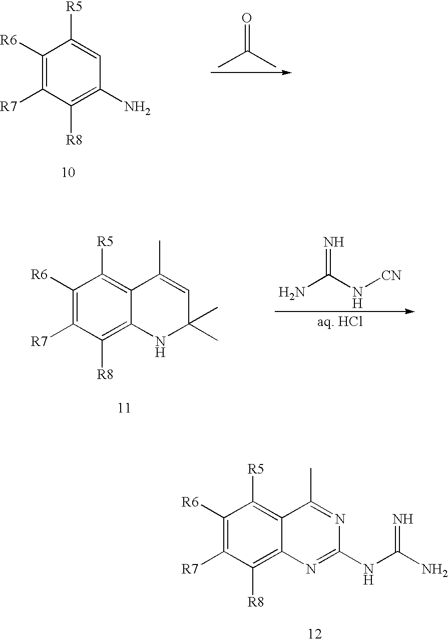

Compound of formula 12 is prepared in a similar manner as described by Webb et al. (Bioorg, Med. Chem., 11, 77-86 (2003)) with modifications. To a solution of aniline of formula X in acetone is added successively t-butylcatechol, iodine and magnesium sulfate. The resulting mixture is stirred and heated to reflux for 1-20 hours. The mixture was cooled to 0-30° C., preferably to room temperature and filtered. The filter cake is washed with acetone. The combined filtrate and washings are evaporated. The product is purified by silica gel chromatography using combination of solvents such as ethyl acetate and hexanes. The product is converted to the hydrochloride salt by dissolving in solvents such as diethyl ether and saturating the solution with hydrochloric acid gas. The precipitate that formed is collected by filtration and dried under vacuum to give compound of formula 11.

Alternatively, the desired intermediate of formula 11 can also be prepared as following: A solution of aniline of formula 10 and scandium trifluoromethanesulfonate in acetone is heated to reflux for 1-96 hours. The solvent was evaporated and the product is purified by silica gel chromatography using combination of solvents such as ethyl acetate and hexanes. The product is converted to the hydrochloride salt by dissolving in solvents such as diethyl ether and saturating the solution with hydrochloric acid gas. The precipitate that formed is collected by filtration and dried under vacuum to give compound of formula 11.

A mixture of the hydrochloride salt of 1,2-dihydroquinoline of formula 11 and dicyandiamide in solvents such as water is stirred and heated to reflux for 1-24 hours. The reaction mixture is filtered while still warm to remove oily by-products, and the filtrate was subsequently treated with solution of sodium hydroxide. A precipitate forms and the mixture is cooled to room temperature. The precipitate is collected by filtration, washed with solvents such as isopropanol and dried. This residue is stirred with solvents such as isopropanol and heated to boiling, then cooled to room temperature. The precipitate is collected by filtration and dried under vacuum to give compound of formula 12.

### Example 5

### General Method E for the Preparation of Quinazolin-2-yl Guanidines

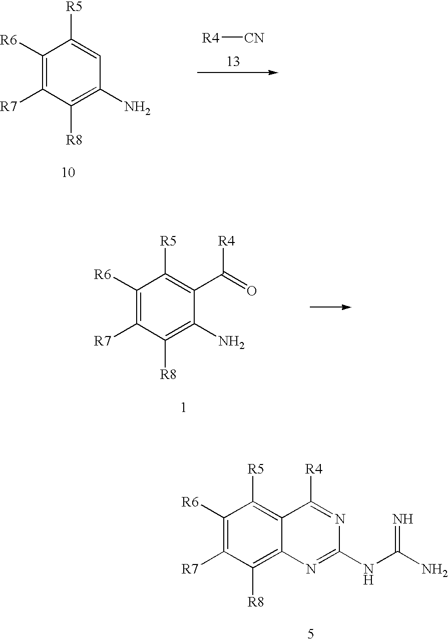

The o-acylanilines of formula 1 are prepared in the similar manner as described by Wu et al. (Synthetic Communications, 29 (20), 3509-3516 (1999)) using nitrile of formula 13 as acylation reagent. The compound of formula 5 is prepared in a similar manner as described by Theiling et al. (J. Am. Chem. Soc., 74, 1834-1836 (1952)). The hydrochloric acid salt of o-acylaniline of formula 13 is mixed with cyanoguanidine in solvents such as THF, preferably THF and heated at between 40-70° C., preferably 60° C. overnight. The solvent is removed by evaporation and the product is washed using solvents such as methanol or ethanol, preferably methanol. The product is filtered and dried under high vacuum to give compound of formula 5.

### Example 6

### General Method F for the Preparation of Isatoic Anhydrides

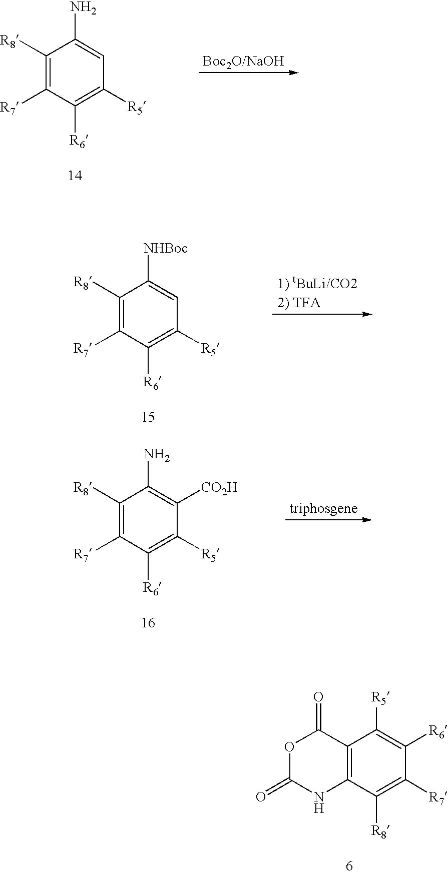

In method F, aniline of formula 14 is dissolved in solvents such as chloroform, dichloromethane, preferably dichloromethane and aqueous solution of sodium hydroxide is added. To the mixture, Carbonic acid di-tert-butyl ester in solvent such as dichloromethane is added and the mixture is stirred for 1-48 hours. The organic layer is separated and the aqueous layer is extracted three times with solvents such as dichloromethane. The combined organic is dried over drying agents such as anhydrous Na₂SO₄ and evaporated to give compound of formula 15.

Compound 16 is prepared in the similar manner as described by Bengtsson et al. (J. Org. Che., 1989, 54, 4549-4553). Compound of formula 15 is dissolved in anhydrous solvents such as tetrahydrofuran and evacuated. The solution is cooled to between −70-0° C. and organic base such as tert-butyl lithium is added. The reaction is stirred at this temperature for 1-10 hours, preferably 2 hours. To slurry of the dry ice (CO₂) in solvents such as tetrahydrofuran, the above solution is transferred. When the reaction reaches room temperature, the reaction is poured into ice water containing acid such as hydrochloric acid and extracted with solvents such as ethyl acetate. The combined organic is dried and concentrated to give an oil. The product is purified by silica gel chromatography using combination of solvents such as ethyl acetate and hexanes to give an intermediate which is subsequently treated with acid such as trifluoroacetic acid (TFA) for between 10-120 minutes and the mixture is evaporated to remove the volatiles. The residue is dried under high vacuum to give compound of formula 16.

Compound of formula 16 is dissolved in solvents such as tetrahydrofuran and triphosgene is added and the reaction is stirred for between 0.5-4 hours. The mixture is poured into ice-water. The precipitate is filtered, washed with water and dried to give isatoic anhydride of formula 6.

### Example 7

### General Method G for the Preparation of Isatoic Anhydrides

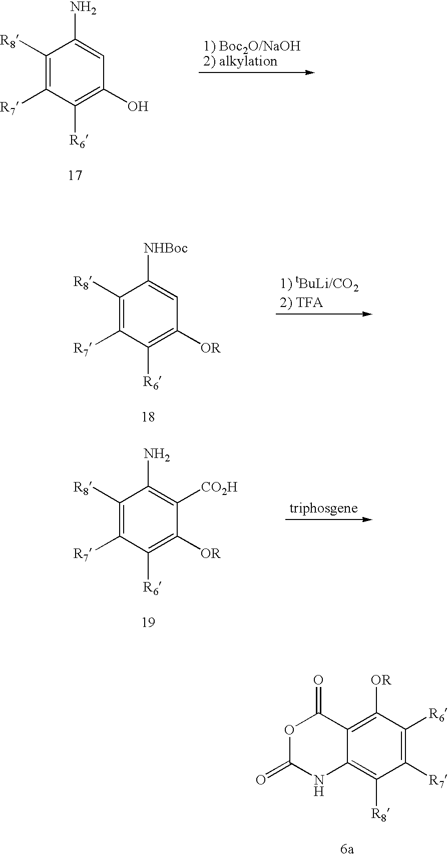

In method G, aniline of formula 17 is dissolved in solvents such as dichloromethane and solution of sodium hydroxide is added. To the mixture, carbonic acid di-tert-butyl ester in solvent such as dichloromethane is added and the mixture is stirred for 1-48 hours, aqueous extraction and the product is dried under high vacuum. The residue obtained is dissolved in solvents such as tetrahydrofuran, alcohol and triphenylphosphine are added followed by addition of diethylazodicarboxylate or diisopropylazodicarboxylate. The reaction is stirred for between 1-24 hours. The product is purified by silica gel chromatography using combination of solvents such as ethyl acetate and hexanes to give compound of formula 18.

Compound 19 is prepared in the similar manner as described by Bengtsson et al. (J. Org. Chem, 1989, 54, 4549-4553). Compound of formula 18 is dissolved in anhydrous solvents such as tetrahydrofuran and evacuated. The solution is cooled to between −70-0° C. and organic base such as tert-butyl lithium is added. The reaction is stirred at this temperature for 1-10 hours, preferably 2 hours. To the slurry of the dry ice in solvents such as tetrahydrofuran, the above solution is transferred. When the reaction reaches room temperature, the reaction is poured into ice water containing acid such as hydrochloric and extracted with solvents such as ethyl acetate, preferably, ethyl acetate. The combined organic is dried and concentrated to give an oil. The product is purified by silica gel chromatography using combination of solvents such as ethyl acetate and hexanes to give the precursor of compound of formula 19. The precursor of compound of formula 19 is treated with acid such as trifluoroacetic acid (TFA) for between 10-120 minutes. The residue is dried under high vacuum to give a compound of formula 19.

Compound of formula 19 is dissolved in solvents such as tetrahydrofuran and triphosgene is added and the reaction is stirred for between 0.5-4 hours. The mixture is poured into ice-water. The precipitate is filtered, washed with water and dried to give isatoic anhydride of formula 6a.

### Example 8

### General Method H for the Preparation of Isatoic Anhydrides

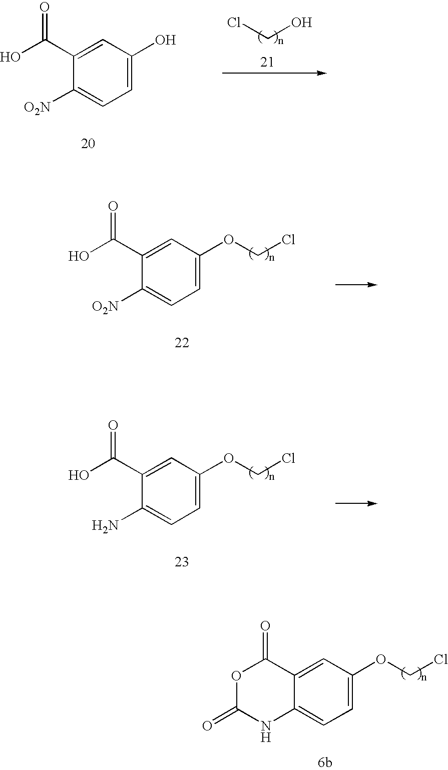

5-hydro-2-nitro-benzoic acid of formula 20 is dissolved in solvents such as tetrahydrofuran, chloroalcohol of formula 21 and triphenylphosphine are added. To the solution, diethylazodicarboxylate or diisopropylazidicarboxylate is added slowly. The reaction is stirred for 1-24 hour. To the reaction, aqueous solution of sodium hydroxide is added and stirred for 1-24 hours. The mixture is extracted with solvents such as dichloromethane. The aqueous layer is filtered by suction and washed with water and the filtrate is acidified using concentrate acid such as hydrochloric acid to pH=2-3. The precipitate is filtered by suction and washed with water and dried under vacuum to give compound of formula 22.

Compound of formula 22 is dissolved in solvents such as methanol, ethanol, ethyl acetate, THF, preferably ethanol and Pd/C is added under argon or nitrogen. The mixture is evacuated and hydrogen atmoshpere applied at ambient pressure. The reaction is stirred between 1-24 hours. The reaction is filtered by suction through glass microfiber filter to remove the Pd/C and washed with solvents such as methanol, ethanol, ethyl acetate, THF, preferably ethanol. The combined filtrate is concentrated to yield compound of formula 23.

Compound of formula 23 is dissolved in solvents such as tetrahydrofuran, triphosgene is added and the reaction is stirred for between 0.5-4 hours. The mixture is poured into ice-water. The precipitate is filtered, washed with water and dried to give isatoic anhydride of formula 6b.

### Example 9

### 2-(6-Methoxy-4-methyl-quinazolin-2-ylamino)-quinazolin-4-ol

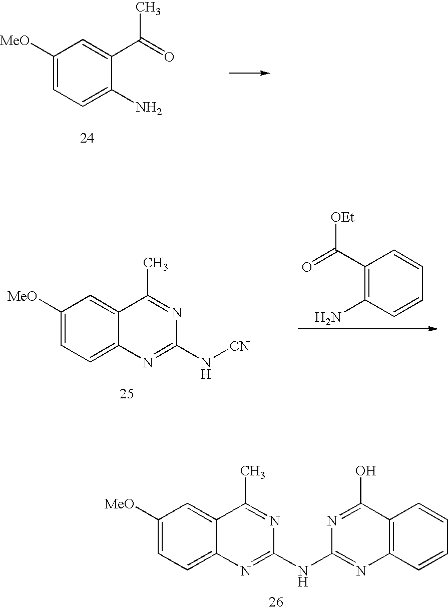

As outlined in method A, Example 1, 2-amino-5-methoxyacetophenone hydrogen chloride of formula 24 (1.2 mmol) is suspended in 10 ml of tetrahydrofuran and sodium dicyanamide (1.0 mmol) is added. The mixture is stirred at 40° C. overnight and filtered and washed with tetrahydrofuran. The product is dried under vacuum to give 6-methoxy-4-methyl-quinazolin-2-yl-cyanamide of formula 25.

6-Methoxy-4-methyl-quinazolin-2-yl-cyanamide of formula 25 (0.1 mmol) is suspended in 2 ml of tetrahydrofuran and ethyl anthranilate (0.1 mmol) and concentrate hydrochloric acid (0.1 mmol) is added. The mixture is stirred at 60° C. overnight under nitrogen flow. The solid material obtained is purified by silica gel chromatography using gradient 0-10% B in 27 minutes (A: dichloromethane, B: 10% triethylamine in methanol) to give compound 26.

¹H NMR (400 MHz, DMSO-d6): δ 13.66 (s, 1H), 11.20 (s, 1H), 8.06 (d, 1H), 7.34 (m, 2H), 7.73 (m, 1H), 7.51 (d, 1H), 7.47 (d, 1H), 7.32 (t, 1H), 3.95 (s, 3H), 2.91 (s, 3H). MS (ES+): MZ 334 (M+1).

### Example 10

### 8-Methoxy-2-(6-methoxy-4-methyl-quinazolin-2-ylamino)-quinazolin-4-ol

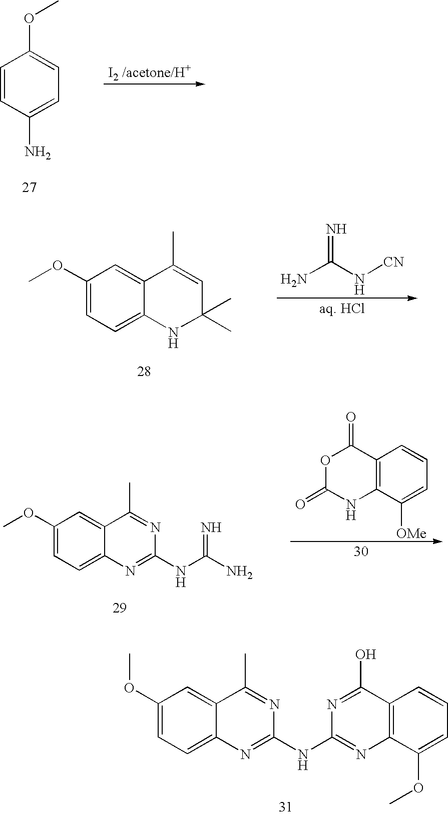

As outlined in general method D, Example 4, to a solution of p-anisidine of formula 27 (16.75 g, 136 mmol) in acetone (230 mL) are added successively t-butylcatechol (0.68 g, 4.1 mmol), iodine (1.73 g, 6.8 mmol), and magnesium sulfate (81.9 g, 680 mmol). The resulting mixture is stirred and heated to reflux for 17.5 hr. The mixture is cooled to room temperature and filtered. The filter cake is washed with acetone. The combined filtrate and washings are evaporated. The product is purified by silica gel chromatography using 10% ethyl acetate in hexanes to give yellow oil. The oil is converted to the hydrochloric acid salt by dissolving in ether (200 mL) and saturating the solution with hydrochloric acid gas. The precipitate that formed is collected by filtration and dried under vacuum to give 13.0 g (40%) of compound of formula 28 as a light beige powder. LCMS (APCI+), m/z 204 (M+1) detected. ¹H NMR (400 mHz, DMSO-d6) δ 7.46 (d, 1H), 6.95 (m, 2H), 5.77 (s, 1H), 3.80 (s, 3H), 2.03 (s, 3H), 1.39 (s, 6H)

A mixture of the hydrochloric acid salt of 6-methoxy-2,2,4-trimethyl-1,2-dihydroquinoline of formula 28 (12.90 g, 53.8 mmol) and dicyandiamide (5.43 g, 64.6 mmol) in water (200 mL) is stirred and heated to reflux for 15 hr. The reaction mixture is gravity filtered hot (80-90° C.) to remove oily by-products, and the filtrate is stirred hot and treated with 1M sodium hydroxide (30 mL). A precipitate formed, and the mixture is cooled to room temperature. The precipitate is collected by filtration, washed with isopropanol, and air-dried to a moist solid. This solid is stirred with isopropanol (200 mL) and heated to boiling, then cooled to room temperature. The precipitate is collected by filtration and dried under vacuum to give 7.86 g (63%) of the compound of formula 29 as a light yellow powder. LCMS (APCI+) m/z 232 (M+1) detected; ¹H NMR (400 MHz, DMSO−d6) δ 7.96 (br. s,2H), 7.71 (d, 1H), 7.50 (dd, 1H), 7.39 (d, 1H), 3.91 (s, 3H), 2.81 (s, 3H).

As outlined in method B, Example 2, compound of formula 29 (1.0 mmol) is dissolved in 2 ml of dimethylformamide, isatoic anhydride of formula 30 (1.10 mmol) and diisopropylethylamine (DIEA, 1.10 mmol) are added. The mixture is stirred at 100° C. overnight. The reaction is cooled to room temperature and the product is filtered and washed with small amount of dimethylformamide, tetrahydrofuran and diethyl ether and dried to give compound 31.

¹H NMR (400 MHz, DMSO-d6): δ 7.76 (d, 1H), 7.68 (m, 1H), 7.59 (m, 1H), 7.54 (d, 1H), 7.40 (d, 1H), 7.32 (t, 1H), 4.08 (s, 3H), 3.96 (s, 3H), 2.95 (s, 3H). MS (ES+): MZ 364 (M+1).

### Example 11

### 2-(6,8-Dimethoxy-4-methyl-quinazolin-2-ylamino)-8-methoxy-quinazolin-4-ol

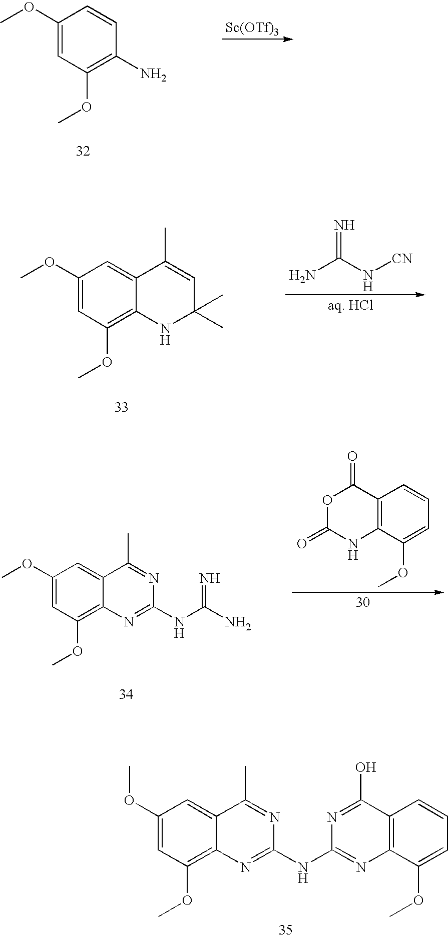

As outlined in method D, Example 4, a solution of 2,4-dimethoxyaniline of formula 32 (15.7 g, 102 mmol) and scandium trifluoromethanesulfonate (5.0 g, 10.2 mmol) in acetone (600 mL) is heated to reflux for three days. The solvent is evaporated. A silica plug is prepared by dissolving the residue in methanol (250 mL) and adding silica gel (150 mL), then evaporating to dryness. The product is purified by silica gel chromatography using 10% ethyl acetate in hexanes to give compound of formula 33 as yellow oil. LCMS (APCI+), m/z 234 (M+1) detected; ¹H NMR (400 MHz, DMSO-d6) δ 6.41 (m, 1H), 6.27 (m, 1H), 5.32 (s, 1H), 4.48 (s, 1H), 3.75 (s, 3H), 3.68 (s, 3H), 1.89 (s, 3H), 1.17 (s, 6H).

A mixture of the hydrochloric acid salt of 6,8-dimethoxy-2,2,4-trimethyl-1,2-dihydroquinoline of formula 33 (53.8 mmol) and dicyandiamide (5.43 g, 64.6 mmol) in water (200 mL) is stirred and heated to reflux for 15 hr. The reaction mixture is gravity filtered hot (80-90° C.) to remove oily by-products, and the filtrate is stirred hot and treated with 1M sodium hydroxide (30 mL). A precipitate formed, and the mixture is cooled to room temperature. The precipitate is collected by filtration, washed with isopropanol, and air-dried to a moist solid. This solid is stirred with isopropanol (200 mL) and heated to boiling, then cooled to room temperature. The precipitate is collected by filtration and dried under vacuum to give 7.86 g (63%) of compound of formula 34 as a light yellow powder.

As outlined in method B, Example 2, compound of formula 34 (1.0 mmol) is dissolved in 2 ml of dimethylformamide, isatoic anhydride of formula 30 (1.10 mmol) and diisopropylethylamine (DIEA, 1.10 mmol) are added. The mixture is stirred at 100° C. overnight. The reaction is cooled to room temperature and the product is filtered and washed with small amount of dimethylformamide, tetrahydrofuran and diethyl ether and dried to give compound 35.

¹H NMR (400 MHz, DMSO-d6): δ 7.55 (d, 1H), 7.28 (m, 2H), 7.02 (s, 1H), 6.96 (s, 1H), 3.99 (s, 3H), 3.95 (s, 3H), 3.88 (s, 3H), 2.83 (s, 3H). MS (ES+): MZ 394 (M+1).

### Example 12

### 1-{2-[4-Methyl-2-(8-methyl-2,3-dihydro-1,4-dioxa-5,7-diaza-phenanthren-6-ylamino)-quinazolin-6-yloxy]-ethyl}-pyrrolidin-3-ol

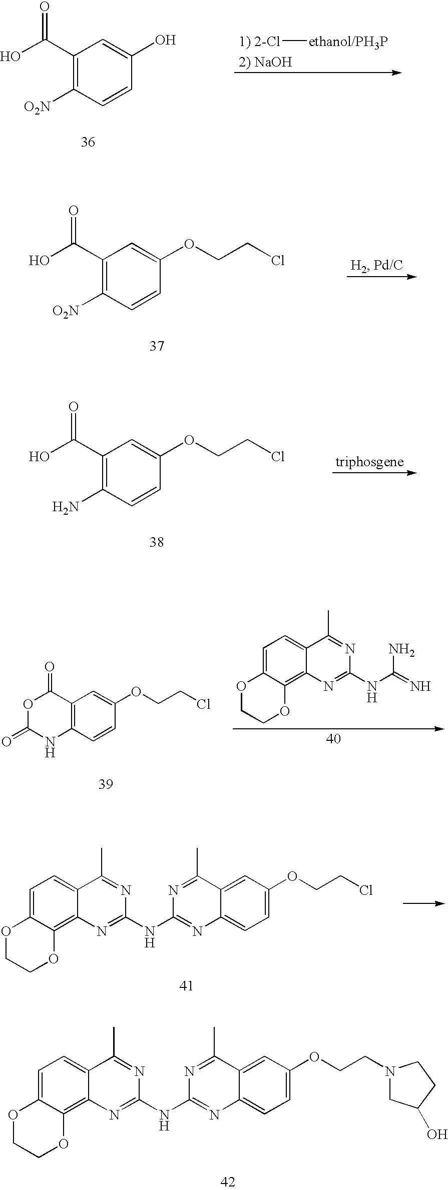

As outlined in method H, 5-hydro-2-nitro-benzoic acid of formula 36 (1.83 g, 10 mmol) is dissolved in tetrahydrofuran (50 ml); 2-chloroethanol (20 mmol) and triphenylphosphine (20 mmol) are added. To the solution, diethylazodicarboxylate (20 mmol) is added slowly. The reaction is stirred for 1 hour. 10 mmol more of 2-chloroethanol, triphenylphosphine and diethylazodicarboxylate are added sequentially. The reaction is stirred at room temperature overnight. To the reaction, 1N sodium hydroxide (50 ml) is added and stirred for 2 hours. The mixture is diluted with water (100 ml) and extracted with dichloromethane three times (3×20 ml). The aqueous layer is filtered by suction and washed with water and the filtrate is acidified using concentrated hydrochloric acid to pH=2-3. The precipitate is filtered by suction and washed with water and dried under vacuum. 1.67 g (68%) of compound of formula 37 is obtained.

Compound of formula 37 (1.67, 6.8 mmol) is dissolved in methanol (20 ml) and 10% Pd/C (200 mg) is added under nitrogen. The mixture is evacuated and hydrogen is applied at 1 atmosphere. The reaction is stirred overnight. The reaction is filtered by suction through glass microfiber filter to remove the Pd/C and washed with methanol. The combined filtrate is concentrated to yield a solid material. 1.47 g (100%) of compound of formula 38 is obtained.

Compound of formula 38 (1.47 g, 6.8 mmol) obtained is dissolved in tetrahydrofuran (20 ml), to which triphosgene (5 mmol) is added. The mixture is heated to reflux for 1 hour. The reaction is poured into ice-water. The precipitate is filtered by suction and washed with water and dried under vacuum. 1.0 g (60%) of compound of formula 39 is obtained.

¹H NMR (400 MHz, DMSO-d6): δ 11.60 (s, 1H), 7.40 (d, 1H), 7.33 (s, 1H), 7.09 (d, 1H), 4.30 (t, 2H), 3.92 (t, 2H). MS (ES−): MZ 240 (M−1).

As outlined in method B, Example 2, to a 5 ml sealable vial is placed a Teflon® stir-bar, N-(8-Methyl-2,3-dihydro-1,4-dioxa-phenanthren-6-yl)-guanidine formula of 40 (1.0 mmol), 6-(2-Chloro-ethoxy)-1H-benzo[d][1,3]oxazine-2,4-dione (1.2 mmol) of formula 39, DIEA (1.20 mmol) and 2.5 ml of dimethylformamide. The vial is capped and put under an atmosphere of N₂ and is stirred at 110° C. overnight. The solid material obtained via filtration is dissolved in 5 ml of trifluoroacetic acid and evaporated, dried in vacuo to give compound of formula 41. ¹H NMR (400 MHz, DMSO-d6): δ 7.69 (d, 1H), 7.41-7.47 (m, 3H), 7.11 (d, 1H), 4.47 (s, 4H), 4.35 (t, 2H), 4.00 (t, 2H), 2.81 (s, 3H). MS (ES+): MZ 440 (M+1).

As outlined in method C, Example 3, a Teflon® stirbar, compound of formula 41 (90.9 μmol) and 1.0 ml NMP to a 2.5 ml is placed in a sealable microwave vial. To this stirring suspension is added 3-pyrrolidinol (0.455 mmol) via syringe. The vial is sealed and heated at 150° C. at 300 W for 30 minutes in the Emrys™ Optimizer, microwave synthesizer, by Personal Chemistry®. The resultant solution is precipitated into 20 ml diethyl ether. The solid material obtained is purified by silica gel chromatography using gradient 0-90% B in 54 minutes (A: dichloromethane, B: Saturated NH₃/methanol in dichloromethane). Product is converted to the trifluoroacetate salt by dissolving in minimum amount of trifluoroacetic acid and precipitating with diethyl ether, the resultant solid is collected and dried on in vacuo to give compound 42. ¹H NMR (400 MHz, DMSO-d6): δ 7.72 (d, 1H), 7.42-7.54 (m, 5H), 7.14 (d, 1H), 4.48 (s, 4H), 4.42 (t, 2H), 3.69 (t, 2H), 3.13-3.54 (m, 5H). MS (ES+): MZ 491 (M+1).

### Example 13

### 2-(6,7-Dimethoxy-4-methyl-quinazolin-2-ylamino)-5-ethoxy-quinazolin-4-ol

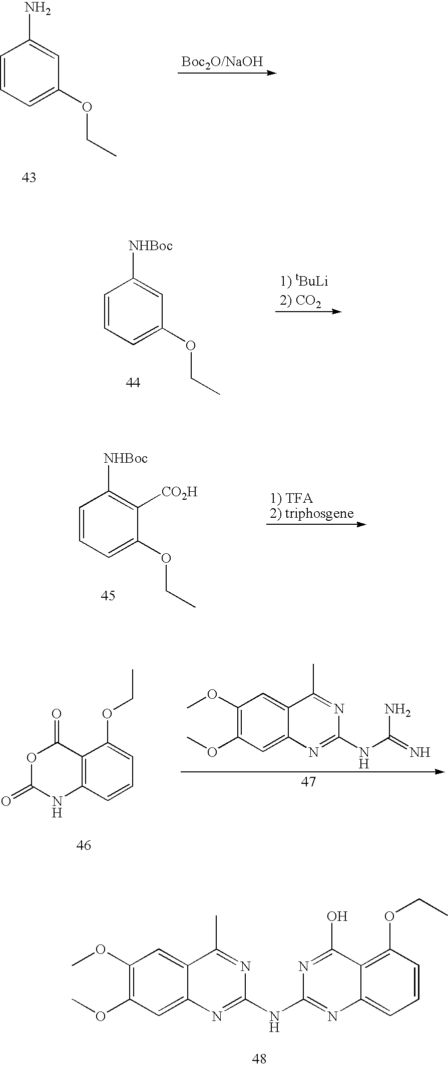

As outlined in method F, 3-Ethoxy-phenylamine of formula 43 (20 mmol) is dissolved in 100 ml of dichloromethane and 40 ml of 1 N Sodium hydroxide is added. To the mixture, 1.5 eq. of Boc₂O in 20 ml dichloromethane is added and the mixture is stirred overnight. The organic layer is separated and the aqueous layer is extracted three times with dichloromethane. The combined organic is dried over Na₂SO₄ and evaporated to give 3-Ethoxy-phenyl)-carbamic acid tert-butyl ester of formula 44.

The (3-Ethoxy-phenyl)-carbamic acid tert-butyl ester of formula 44 (10 mmol) is dissolved in dry tetrahydrofuran (20 ml) and evacuated. The solution is cooled to −20° C. and 2.5 eq. of ^{t}BuLi (1.7 M in pentane). The reaction is stirred at this temperature for 2 hours. To slurry of dry ice in tetrahydrofuran, the lithiation solution is transferred. The reaction vessel should be opened to ensure the release of carbon dioxide. When the reaction reaches room temperature, the reaction is poured into 30 ml of 1M hydrochloric acid in 100 g of ice and extracted with ethyl acetate three times. The combined organic is dried and concentrated to give an oil. The crude is purified by silica gel chromatography using 0-50% B gradient (A: hexanes, B: ethyl acetate). Compound of formula 45 is obtained in 40% yield.

2-tert-Butoxycarbonylamino-6-ethoxy-benzoic acid of formula 45 obtained is treated with 10 ml trifluoroacetic acid (TFA) for 30 min. and monitored by LC/MS to ensure the completion of the reaction. When the reaction is completed, trifluoroacetic acid is evaporated. The residue is re-dissolved in dichloromethane and evaporated. The residue is dried under high vacuum to obtain 2-amino-6-ethoxy-benzoic acid. The 2-amino-6-ethoxy-benzoic acid obtained (2 mmol) is dissolved in 10 ml tetrahydrofuran and 0.7 eq. of triphosgene is added and the reaction is stirred for 2 hours. The reaction is monitored by LC/MS to ensure the completion of the reaction. When the reaction is completed, the mixture is poured into 100 ml of ice-water. The precipitate is filtered, washed with water and dried to give compound of formula 46.

As outlined in method B, Example 2, compound of formula 47 (1.0 mmol) is dissolved in 2 ml of dimethylformamide, isatoic anhydride of formula 46 (1.10 mmol) and diisopropylethylamine (DIEA, 1.10 mmol) are added. The mixture is stirred at 100° C. overnight. The reaction is cooled to room temperature and the product is filtered and washed with small amount of dimethylformamide, tetrahydrofuran and diethyl ether and dried to give compound 48.

¹H NMR (400 MHz, DMSO-d6): δ 7.59 (t, 1H), 7.41 (s, 1H), 7.14 (s, 1H), 7.02 (d, 1H), 6.83 (d, 1H), 4.12 (m, 1H), 4.02 (s, 3H), 3.95 (s, 3H), 2.88 (s, 3H), 1.39 (t, 3H). MS (ES+): MZ 408 (M+1).

### Example 14

### 5-Butoxy-2-(6,7-dimethoxy-4-methyl-quinazolin-2-ylamino)-quinazolin-4-ol

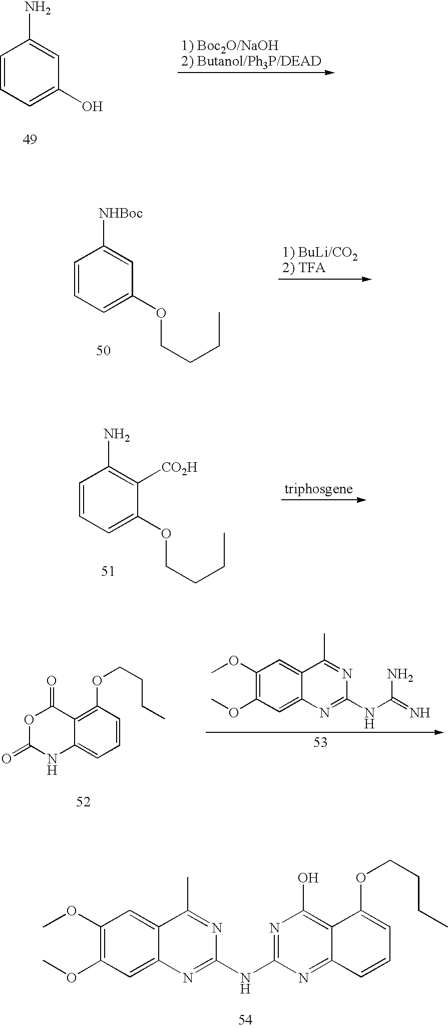

As outlined in method G, 3-hydroxyanaline of formula 49 (20 mmol) is dissolved in 100 ml of dichloromethane and 40 ml of 1 N Sodium hydroxide is added. To the mixture, 1.5 eq. of Boc₂O in 20 ml dichloromethane is added and the mixture is stirred overnight. The organic layer is separated and the aqueous layer is extracted three times with dichloromethane. The combined organic is dried over Na₂SO₄ and evaporated to give oil. (3-Hydroxy-phenyl)-carbamic acid tert-butyl ester (1.83 g, 10 mmol) is dissolved in tetrahydrofuran (50 ml), butanol (10 mmol) and triphenylphosphine (10 mmol) are added. To the solution, diethylazodicarboxylate (10 mmol) is added slowly. The reaction is stirred for 1 hour. 5.0 mmol more of butanol, triphenylphosphine and diethylazodicarboxylate are added sequentially. The reaction is stirred at room temperature overnight. The product is purified by silica gel chromatography using 0-50% B gradient (A: hexanes, B: ethyl acetate) to obtain (3-Butoxy-phenyl)-carbamic acid tert-butyl ester of formula 50.

(3-Butoxy-phenyl)-carbamic acid tert-butyl ester of formula 50 (5 mmol) is dissolved in dry tetrahydrofuran (20 ml) and evacuated. The solution is cooled to −20° C. and 2.5 eq. of ^{t}BuLi (1.7 M in pentane). The reaction is stirred at this temperature for 2 hours. To slurry of dry ice in tetrahydrofuran, the lithiation solution is transferred. The reaction vessel should be opened to ensure the release of carbon dioxide. When the reaction reaches room temperature, the mixture is poured into 15 ml 1N hydrochloric acid in 100 g of ice and extracted with ethyl acetate three times. The combined organic is dried and concentrated to give an oil. The crude is purified by silica gel chromatography using 0-50% B gradient (A: hexanes, B: ethyl acetate) to obtain 2-Butoxy-6-tert-butoxycarbonylamino-benzoic acid in 25% yield. 2-Butoxy-6-tert-butoxycarbonylamino-benzoic acid is treated with 5 ml of trifluoroacetic acid for 30 minutes and monitored by LC/MS to ensure the completion of the reaction. When the reaction is completed, trifluoroacetic acid is evaporated. The residue is re-dissolved in dichloromethane and evaporated. The residue is dried under high vacuum to obtain 2-Amino-6-butoxy-benzoic acid of formula 51.

The 2-Amino-6-butoxy-benzoic acid of formula 51 obtained (1.25 mmol) is dissolved in 10 ml tetrahydrofuran and 0.7 equivalent of triphosgene is added and the reaction is stirred for 2 hours. The reaction is monitored by LC-MS to ensure the completion of the reaction. When the reaction is completed, the mixture is poured into 100 ml of ice-water. The precipitate is filtered, washed with water and dried to give compound of formula 52.

As outlined in method B, Example 2, compound of formula 53 (1.0 mmol) is dissolved in 2 ml of dimethylformamide, isatoic anhydride of formula 52 (1.10 mmol) and diisopropylethylamine (DIEA, 1.10 mmol) are added. The mixture is stirred at 100° C. overnight. The reaction is cooled to room temperature and the product is filtered and washed with small amount of dimethylformamide, tetrahydrofuran and diethyl ether and dried to give compound 54.

¹H NMR (400 MHz, DMSO-d6): δ 7.59 (t, 1H), 7.39 (s, 1H), 7.14 (s, 1H), 7.00 (d, 1H), (d, 1H), 6.82 (d, 1H), 4.05 (t, 1H), 3.96 (s, 3H), 3.94 (s, 3H), 2.87 (s, 3H), 1.76 (m, 2H), 1.55 (m, 2H), 1.07 (t, 3H). MS (ES+): MZ 436 (M+1).

### Example 15

### 2-(8-Methyl-[1,3]dioxolo[4,5-g]quinazolin-6-ylamino)-quinazolin-4-ol

This compound is prepared using method B, Example 2. ¹H NMR (400 MHz, DMSO-d6): δ 13.52 (s, 1H), 11.00 (s, 1H), 8.04 (d, 1H), 7.71 (t, 1H), 7.58 (s, 1H), 7.45 (d, 1H), 7.32 (t, 1H), 7.15 (s, 1H), 6.27 (s, 2H), 2.79 (s, 3H). MS (ES+): MZ 348 (M+1).

### Example 16

### 2-(4-Hydroxy-quinazolin-2-ylamino)-4-methyl-quinazolin-6-ol

This compound is prepared using method B, Example 2. ¹H NMR (400 MHz, DMSO-d6): δ 13.69 (s, 1H), 11.07 (s, 1H), 10.19 (s, 1H), 8.05 (d, 1H), 7.70 (m, 2H), 7.52 (d, 1H), 7.44 (d, 1H), 7.37 (s, 1H), 7.30 (t, 1H), 2.91 (s, 3H). MS (ES+): MZ 320 (M+1).

### Example 17

### (6-Methoxy-4-methyl-quinazolin-2-yl)-(4-methoxy-quinazolin-2-yl)-methyl-amine

This compound is prepared using method B, Example 2. ¹H NMR (400 MHz, DMSO-d6): δ 8.19 (d, 1H), 7.84 (t, 1H), 7.76 (m, 2H), 7.55 (t, 1H), 7.50 (d, 1H), 7.50 (d, 1H), 7.40 (s, 1H), 3.90 (s, 3H), 3.58 (s, 3H), 3.32 (s, 3H), 2.75 (s, 3H). MS (ES+): MZ 362 (M+1).

### Example 18

### 6,7,8-Trimethoxy-2-(6-methoxy-4-methyl-quinazolin-2-ylamino)-quinazolin-4-ol trifluoroacetate salt

This compound is prepared using method B, Example 2. ¹H NMR (400 MHz, DMSO-d6): δ 13.49 (s, 1H), 11.00 (s, 1H), 7.72 (d, 1H), 7.69 (d, 1H), 7.49 (s, 1H), 7.28 (s, 1H), 4.00 (s, 3H), 3.94 (s, 3H), 3.88 (s, 3H), 3.87 (s, 3H), 2.92 (s, 3H). MS (ES+): MZ 424 (M+1).

### Example 19

### 2-(6-Methoxy-4-methyl-quinazolin-2-ylamino)-6,8-dimethyl-quinazolin-4-ol

This compound is prepared using method B, Example 2. ¹H NMR (400 MHz, DMSO-d6): δ 13.51 (s, 1H), 10.85 (s, 1H), 7.72 (d, 1H), 7.68 (s, 1H), 7.60 (d, 1H), 7.48 (s, 1H), 7.41 (s, 1H), 3.94 (s, 3H), 2.90 (s, 3H), 2.46 (s, 3H), 2.36 (s, 3H), MS (ES+): MZ 362 (M+1).

### Example 20

### 2-(6-Methoxy-quinazolin-2-ylamino)-8-methyl-quinazolin-4-ol

This compound is prepared using method B, Example 2. ¹H NMR (400 MHz, DMSO-d6): δ 9.43 (s, 1H), 7.88 (d, 1H), 7.75 (d, 1H), 7.64 (d, 1H), 7.57 (d, 1H), 7.51 (s, 1H), 7.19 (t, 1H), 3.90 (s, 3H), MS (ES+): MZ 334 (M+1).

### Example 21

### 2-(6-Methoxy-4-methyl-quinazolin-2-ylamino)-8-methyl-6-nitro-quinazolin-4-ol

This compound is prepared using method B, Example 2. ¹H NMR (400 MHz, DMSO-d6): δ 8.59 (s, 1H), 8.33 (s, 1H), 7.95 (s, 1H), 7.75 (d, 1H), 7.63 (d, 1H), 3.96 (s, 3H), 2.94 (s, 3H), 2.58 (s, 3H). MS (ES+): MZ 393 (M+1).

### Example 22

### 6-Amino-2-(6-methoxy-4-methyl-quinazolin-2-ylamino)-8-methyl-quinazolin-4-ol trifluoroacetate salt

This compound is prepared using method B, Example 2. ¹H NMR (400 MHz, DMSO-d6): δ 7.73 (d, 1H), 7.64 (m, 1H), 7.51 (d, 1H), 7.44 (s, 1H), 7.23(s, 1H), 3.94 (s, 3H), 2.93 (s, 3H), 2.52 (s, 3H). MS (ES+): MZ 363 (M+1).

### Example 23

### 6-N-methylamino-2-(6-methoxy-4-methyl-quinazolin-2-ylamino)-8-methyl-quinazolin-4-ol trifluoroacetate salt

This compound is prepared using method B, Example 2. ¹H NMR (400 MHz, DMSO-d6): δ 7.72 (d, 1H), 7.63 (d, 1H), 7.50 (s, 1H), 7.02 (s, 1H), 6.94 (s, 1H), 3.94 (s, 3H), 3.75 (s, 3H), 2.91 (s, 3H), 2.76 (s, 3H), MS (ES+): MZ 377 (M+1).

### Example 24

### 6-N-dimethylamino-2-(6-methoxy-4-methyl-quinazolin-2-ylamino)-8-methyl-quinazolin-4-ol trifluoroacetate salt

This compound is prepared using method B, Example 2. ¹H NMR (400 MHz, DMSO-d6): δ 7.72 (d, 1H), 7.63 (d, 1H), 7.50 (s, 1H), 7.25 (s, 1H), 7.07 (s, 1H), 3.94 (s, 3H), 2.97 (s, 6H), 2.92 (s, 3H), 2.54 (s, 3H), MS (ES+): MZ 391 (M+1).

### Example 25

### 7-Fluoro-2-(6-methoxy-4-methyl-quinazolin-2-ylamino)-6-nitro-quinazolin-4-ol trifluoroacetate salt

This compound is prepared using method B, Example 2. ¹H NMR (400 MHz, DMSO-d6): δ 8.34 (s, 1H), 7.75 (d, 1H), 7.62 (m, 1H), 7.49 (d, 1H), 6.80 (s, 1H), 3.94 (s, 3H), 2.91 (s, 3H). MS (ES+): MZ 397 (M+1).

### Example 26

### 2-(8-Methoxy-4-methyl-5-nitro-quinazolin-2-ylamino)-quinazolin-4-ol trifluoroacetate salt

This compound is prepared using method B, Example 2. ¹H NMR (400 MHz, DMSO-d6): δ 8.06 (d, 1H), 8.04 (d, 1H), 7.75 (t, 1H), 7.47 (d, 1H), 7.45 (d, 1H), 7.35 (t, 1H), 4.14 (s, 3H), 2.63 (s, 3H). MS (ES+): MZ 379 (M+1).

### Example 27

### 2-(5,8-Dimethoxy-4-methyl-quinazolin-2-ylamino)-quinazolin-4-ol trifluoroacetate salt

This compound is prepared using method B, Example 2. ¹H NMR (400 MHz, DMSO-d6): δ 8.06 (d, 1H), 7.74 (t, 1H), 7.44 (d, 1H), 7.38 (d, 1H), 7.36 (t, 1H), 6.91 (d, 1H), 3.99 (s, 3H), 3.92 (s, 3H), 2.97 (s, 3H). MS (ES+): MZ 364 (M+1).

### Example 28

### 2-(5,6,7-Trimethoxy-4-methyl-quinazolin-2-ylamino)-quinazolin-4-ol trifluoroacetate salt

This compound is prepared using method B, Example 2. ¹H NMR (400 MHz, DMSO-d6): δ 8.06 (d, 1H), 7.74 (t, 1H), 7.50 (d, 1H), 7.33 (t, 1H), 7.02 (s, 1H), 4.04 (s, 3H), 4.01 (s, 3H), 3.84 (s, 3H), 2.96 (s, 3H). MS (ES+): MZ 394 (M+1).

### Example 29

### 2-(5-Methoxy-4,8-dimethyl-quinazolin-2-ylamino)-quinazolin-4-ol trifluoroacetate salt

This compound is prepared using method B, Example 2. ¹H NMR (400 MHz, DMSO-d6): δ 8.05 (d, 1H), 7.74 (t, 1H), 7.73 (d, 1H), 7.46 (d, 1H), 7.32 (t, 1H), 6.92 (d, 1H), 3.94 (s, 3H), 2.96 (s, 3H), 2.58 (s, 3H). MS (ES+): MZ 348 (M+1).

### Example 30

### 2-(8-Methoxy-4-methyl-5-nitro-quinazolin-2-ylamino)-6,8-dimethyl-quinazolin-4-ol, trifluoroacetate salt

This compound is prepared using method B, Example 2. ¹H NMR (400 MHz, DMSO-d6): δ 7.98 (d, 1H), 7.64 (s, 1H), 7.42 (d, 1H), 7.40 (s, 1H), 4.10 (s, 3H), 2.61 (s, 3H), 2.48 (s, 3H), 2.35 (s, 3H). MS (ES+): MZ 407 (M+1).

### Example 31

### 4-Hydroxy-2-(6-methoxy-4-methyl-quinazolin-2-ylamino)-quinazoline-7-carboxylic acid

This compound is prepared using method B, Example 2. ¹H NMR (400 MHz, DMSO-d6): δ 13.72 (s, 1H), 11.23 (s, 1H), 8.09 (d, 1H), 7.91 (s, 1H), 7.74 (d, 1H), 7.72 (d, 1H), 7.58 (m, 1H), 7.46 (s, 1H), 3.91 (s, 3H), 2.88 (s, 3H). MS (ES+): MZ 378 (M+1).

### Example 32

### 2-(7-Fluoro-6-methoxy-4-methyl-quinazolin-2-ylamino)-quinazolin-4-ol trifluoroacetate salt

This compound is prepared using method B, Example 2. ¹H NMR (400 MHz, DMSO-d6): δ 8.03 (d, 1H), 7.73 (t, 1H), 7.68 (s, 1H), 7.65 (s, 1H), 7.51 (d, 1H), 7.32 (t, 1H), 4.00 (s, 3H), 2.90 (s, 3H). MS (ES+): MZ 352 (M+1).

### Example 33

### 6-Chloro-2-(6-methoxy-4-methyl-quinazolin-2-ylamino)-quinazolin-4-ol

This compound is prepared using method B, Example 2. ¹H NMR (400 MHz, DMSO-d6): δ 13.75 (s, 1H), 11.26 (s, 1H), 7.95 (s, 1H), 7.71 (d, 2H), 7.60 (d, 1H), 7.45 (m, 2H), 3.92 (s, 3H), 2.89 (s, 3H). MS (ES+): MZ 368 (M+1).

### Example 34

### 2-[(6-Methoxy-4-methyl-quinazolin-2-yl)-methyl-amino]-quinazolin-4-ol

This compound is prepared using method B, Example 2. ¹H NMR (400 MHz, DMSO-d6): δ 8.04 (d, 1H), 7.73 (m, 2H), 7.63 (d, 1H), 7.53 (d, 1H), 7.49 (s, 1H), 7.34 (t, 1H), 3.94 (s, 3H), 3.83 (s, 3H), 2.94 (s, 3H). MS (ES+): MZ 348 (M+1).

### Example 35

### 2-(4-Methyl-6-methylsulfanyl-quinazolin-2-ylamino)-quinazolin-4-ol trifluoroacetate salt:

This compound is prepared using method B, Example 2. ¹H NMR (400 MHz, DMSO-d6): δ 8.07 (d, 1H), 7.88 (m, 2H), 7.75 (m, 2H), 7.51 (d, 1H), 7.35 (t, 1H), 2.93 (s, 3H), 2.45 (s, 3H). MS (ES+): MZ 350 (M+1).

### Example 36

### 6,8-Dimethyl-2-(5 6,7-trimethoxy-4-methyl-quinazolin-2-ylamino)-quinazolin-4-ol trifluoroacetate salt

This compound is prepared using method B, Example 2. ¹H NMR (400 MHz, DMSO-d6): δ 7.57 (s, 1H), 7.39 (s, 1H), 6.86 (s, 1H), 4.00 (s, 3H), 3.99 (s, 3H), 3.84 (s, 3H), 2.90 (s, 3H), 2.45 (s, 3H), 2.32 (s, 3H). MS (ES+): MZ 422 (M+1).

### Example 37

### 5-Methoxy-2-(6-methoxy-4-methyl-quinazolin-2-ylamino)-quinazolin-4-ol trifluoroacetate salt

This compound is prepared using method B, Example 2. ¹H NMR (400MHz, DMSO-d6): 6 7.75 (d, 1H), 7.63 (m, 2H), 7.51 (s, 1H), 7.04 (d, 1H), 6.86 (d, 1H), 3.94 ( 3.87 (s, 3H), 2.93 (s, 3H). MS (ES+): MZ 364 (M+1).

### Example 38

### N-[4-Hydroxy-2-(6-methoxy-4-methyl-quinazolin-2-ylamino)-quinazolin-6-yl]-acetamide trifluoroacetate salt:

This compound is prepared using method B, Example 2. ¹H NMR (400 MHz, DMSO-d6): δ 10.18 (s, 1H), 8.38 (d, 1H), 7.86 (m, 1H), 7.77 (d, 1H), 7.64 (m, 1H), 7.51 (s, 1H), 7.47 (d, 1H), 3.94 (s, 3H), 2.92 (s, 3H), 2.08 (s, 3H). MS (ES+): MZ 391 (M+1).

### Example 39

### 8-Bromo-2-(6-methoxy-4-methyl-quinazolin-2-ylamino)-6-methyl-quinazolin-4-ol trifluoroacetate salt:

This compound is prepared using method B, Example 2. ¹H NMR (400 MHz, DMSO-d6): δ 7.91 (s, 1H), 7.85 (s, 1H), 7.80 (d, 1H), 7.62 (m, 1H), 7.51 (s, 1H), 3.94 (s, 3H), 2.93 (s, 3H), 2.40 (s, 3H). MS (ES+): MZ 427 (M+1).

### Example 40

### 2-(5-Methoxy-4,8-dimethyl-quinazolin-2-ylamino)-6,8-dimethyl-quinazolin-4-ol trifluoroacetate salt

This compound is prepared using method B, Example 2. ¹H NMR (400 MHz, DMSO-d6): δ 7.67 (d, 1H), 7.64 (s, 1H), 7.39 (s, 1H), 6.87 (d, 1H), 3.93 (s, 3H), 2.93 (s, 3H), 2.53 (s, 3H), 2.45 (s, 3H), 2.34 (s, 3H). MS (ES+): MZ 376 (M+1).

### Example 41

### 2-(6,7-Dimethoxy-4-methyl-quinazolin-2-ylamino)-6,8-dimethyl-quinazolin-4-ol trifluoroacetate salt

This compound is prepared using method B, Example 2. ¹H NMR (400 MHz, DMSO-d6): δ 7.66 (s, 1H), 7.44 (s, 1H), 7.40 (s, 1H), 7.10 (s, 1H), 4.01 (s, 3H), 3.95 (s, 3H), 2.87 (s, 3H), 2.36 (m, 6H). MS (ES+): MZ 392 (M+1).

### Example 42

### 2-(2,2-Difluoro-8-methyl-[1,3]dioxolo[4,5-g]quinazolin-6-ylamino)-quinazolin-4-ol trifluoroacetate salt

This compound is prepared using method B, Example 2. ¹H NMR (400 MHz, DMSO-d6): δ 8.61 (s, 1H), 8.15 (s, 1H), 7.98 (s, 1H), 7.77 (s, 1H), 7.37 (s, 1H), 6.80 (d, 1H), 2.48 (s, 3H). MS (ES+): MZ 384 (M+1).

### Example 43

### 2-(5,8-Dimethoxy-4-methyl-quinazolin-2-ylamino)-6,8-dimethyl-quinazolin-4-ol trifluoroacetate salt

This compound is prepared using method B, Example 2. ¹H NMR (400 MHz, DMSO-d6): δ 7.64 (s, 1H), 7.40 (s, 1H), 7.34 (d, 1H), 6.88 (d, 1H), 3.93 (s, 3H), 3.90 (s, 3H), 2.93 (s, 3H), 2.48 (s, 3H), 2.34 (s, 3H). MS (ES+): MZ 392 (M+1).

### Example 44

### 2-(7-Fluoro-6-methoxy-4-methyl-quinazolin-2-ylamino)-6,8-dimethyl-quinazolin-4-ol acetate salt

This compound is prepared using method B, Example 2. ¹H NMR (400 MHz, DMSO-d6): δ 7.69 (m, 2H), 7.61 (d, 1H), 7.44 (s, 1H), 4.04 (s, 3H), 2.92 (s, 3H), 2.36 (s, 6H). MS (ES+): MZ 380 (M+1).

### Example 45

### 2-(4-Fluoromethyl-6-methoxy-quinazolin-2-ylamino)-quinazolin-4-ol trifluoroacetate salt

This compound is prepared using method B, Example 2. ¹H NMR (400 MHz, DMSO-d6): δ 8.07 (d, 1H), 7.85 (d, 1H), 7.75 (t, 1H), 7.68 (m, 1H), 7.50 (d, 1H), 7.46 (s, 1H), 7.35 (t, 1H), 6.17 (s, 1H), 6.06 (s, 1H), 3.94 (s, 3H). MS (ES+): MZ 352 (M+1).

### Example 46

### 2-(6,8-Dimethoxy-4-methyl-quinazolin-2-ylamino)-8-methyl-quinazolin-4-ol trifluoroacetate salt

This compound is prepared using method B, Example 2. ¹H NMR (400 MHz, DMSO-d6): δ 7.87 (s, 1H), 7.59 (s, 1H), 7.22 (s, 1H), 7.04 (s, 1H), 6.97 (s, 1H), 3.98 (s, 3H), 3.90 (s, 3H), 2.85 (s, 3H), 2.51 (s, 3H). MS (ES+): MZ 378 (M+1).

### Example 47

### 6-Amino-2-(6-methoxy-4-methyl-quinazolin-2-ylamino)-quinazolin-4-ol trifluoroacetate salt

This compound is prepared using method B, Example 2. ¹H NMR (400 MHz, DMSO-d6): δ 7.71 (d, 1H), 7.60 (m, 1H), 7.48 (d, 1H), 7.23(d, 1H), 7.19 (d, 1H), 7.05 (m, 1H), 3.94 (s, 3H), 2.88 (s, 3H). MS (ES+): MZ 349 (M+1).

### Example 48

### 2-(8-Methoxy-4-methyl-quinazolin-2-ylamino)-6,8-dimethyl-quinazolin-4-ol

This compound is prepared using method B, Example 2. ¹H NMR (400 MHz, DMSO-d6): δ 11.09 (s, 1H), 7.74 (d, 1H), 7.69 (s, 1H), 7.48 (d, 1H), 7.45 (d, 1H), 7.43 (s, 1H), 4.03 (s, 3H), 2.88 (s, 3H), 2.48 (s, 3H), 2.37 (s, 3H). MS (ES+): MZ 362 (M+1).

### Example 49

### 2-(4-Ethyl-6-methoxy-quinazolin-2-ylamino)-quinazolin-4-ol

This compound is prepared using method B, Example 2. ¹H NMR (400 MHz, DMSO-d6): δ 13.63 (s,1H), 11.08 (s, 1H), 8.04 (d, 1H), 7.72 (m, 2H), 7.61 (m, 1H), 7.52 (s, 1H), 7.47 (d, 1H), 7.31 (t, 1H), 3.94 (s, 3H), 3.33 (m, 2H), 1.43 (t, 3H). MS (ES+): MZ 348 (M+1).

### Example 50

### 2-(6-Methoxy-4-propyl-quinazolin-2-ylamino)-quinazolin-4-ol

This compound is prepared using method B, Example 2. ¹H NMR (400 MHz, DMSO-d6): δ 13.61 (s,1H), 11.08 (s, 1H), 8.05 (d, 1H), 7.72 (m, 2H), 7.62 (m, 1H), 7.53 (s, 1H), 7.47 (d, 1H), 7.31 (t, 1H), 3.95 (s, 3H), 3.33 (m, 2H), 1.92 (m, 2H), 1.07 (t, 3H). MS (ES+): MZ 362 (M+1).

### Example 51

### 2-(8-Methoxy-4-methyl-quinazolin-2-ylamino)-8-methyl-quinazolin-4-ol

This compound is prepared using method B, Example 2. ¹H NMR (400 MHz, DMSO-d6): δ 11.19 (s, 1H), 7.90 (d, 1H), 7.74 (d, 1H), 7.69 (d, 1H), 7.47 (m, 2H), 7.20 (t, 1H), 4.03 (s, 3H), 3.89 (s, 3H), 2.50 (s, 3H). MS (ES+): MZ 348 (M+1).

### Example 52

### 2-(6,7-Dimethoxy-4-methyl-quinazolin-2-ylamino)-quinazolin-4-ol:

This compound is prepared using method B, Example 2. ¹H NMR (400 MHz, DMSO-d6): δ 13.61 (s, 1H), 10.94 (s, 1H), 8.04 (s, 1H), 7.71(s, 1H), 7.41 (s, 2H), 7.30 (s, 1H), 7.11 (s, 1H), 4.02(s, 3H), 3.95 (s, 3H), 2.84 (s, 3H). MS (ES+): MZ 364 (M+1).

### Example 53

### 2-(6-Methoxy-4-methyl-quinazolin-2-ylamino)-6-nitro-quinazolin-4-ol

This compound is prepared using method B, Example 2. ¹H NMR (400 MHz, DMSO-d6): δ 13.85 (s, 1H), 11.56 (s, 1H), 8.65 (s, 1H), 8.37 (d, 1H), 7.69 (d, 1H), 7.58 (m, 1H), 7.48 (d, 1H), 7.42 (t, 1H), 3.90 (s, 3H), 2.88 (s, 3H). MS (ES+): MZ 379 (M+1).

### Example 54

### 2-(6-Methoxy-4-methyl-quinazolin-2-ylamino)-7-nitro-quinazolin-4-ol

This compound is prepared using method B, Example 2. ¹H NMR (400 MHz, DMSO-d6):δ 13.82, (s, 1H), 11.37 (s, 1H), 8.18 (d, 1H), 7.98 (s, 1H), 7.93 (d, 1H), 7.67 (d, 1H), 7.56 (d, 1H), 7.40 (s, 1H), 3.89 (s, 3H), 2.87 (s, 3H). MS (ES+): MZ 379 (M+1).

### Example 55

### 5-Fluoro-2-(6-methoxy-4-methyl-quinazolin-2-ylamino-quinazolin-4-ol

This compound is prepared using method B, Example 2. ¹H NMR (400 MHz, DMSO-d6): δ 13.54 (s, 1H), 11.19 (s, 1H), 7.67 (m, 2H), 7.60 (m, 1H), 7.47 (d, 1H), 7.23 (d, 1H), 7.01 (t, 1H), 3.92 (s, 3H), 2.89 (s, 3H). MS (ES+): MZ 352 (M+1).

### Example 56

### 6-Fluoro-2-(6-methoxy-4-methyl-quinazolin-2-ylamino)-quinazolin-4-ol

This compound is prepared using method B, Example 2. ¹H NMR (400 MHz, DMSO-d6): δ 13.68, (s, 1H), 11.10 (s, 1H), 7.70 (d, 1H), 7.68 (d, 1H), 7.59 (m, 2H), 7.48 (s, 1H), 7.44 (d, 1H), 3.91 (s, 3H), 2.88 (s, 3H). MS (ES+): MZ 352 (M+1).

### Example 57

### 7-Fluoro-2-(6-methoxy-4-methyl-quinazolin-2-ylamino)-quinazolin-4-ol

This compound is prepared using method B, Example 2. ¹H NMR (400 MHz, DMSO-d6): δ 13.62 (s, 1H), 11.23 (s, 1H), 8.81 (m, 1H), 7.72 (d, 1H), 7.60 (m, 1H), 7.47 (s, 1H), 7.14 (t, 2H), 3.92 (s, 3H), 2.89 (s, 3H). MS (ES+): MZ 352 (M+1).

### Example 58

### 2-(8-Methoxy-4-methyl-quinazolin-2-ylamino)-quinazolin-4-ol

This compound is prepared using method B, Example 2. ¹H NMR (400 MHz, DMSO-d6): δ 11.36 (s, 1H), 8.05 (d, 1H), 7.73 (d, 1H), 7.71 (d, 1H), 7.47 (s, 1H), 7.44 (m, 2H), 7.33 (t, 1H), 4.03 (s, 3H), 2.88 (s, 3H). MS (ES+): MZ 334 (M+1).

### Example 59

### 2-(8-Ethoxy-4-methyl-quinazolin-2-ylamino)-quinazolin-4-ol

This compound is prepared using method B, Example 2. ¹H NMR (400 MHz, DMSO-d6): δ 11.36 (s, 1H), 8.07 (d, 1H), 7.71 (m, 2H), 7.44 (m, 2H), 7.41 (s, 1H), 7.33 (t, 1H), 4.25 (s, 2H), 2.87 (s, 3H), 1.61 (s, 3H). MS (ES+): MZ 348 (M+1).

### Example 60

### 2-(6-Methoxy-4-methyl-quinazolin-2-ylamino)-quinazoline-4,6-diol

This compound is prepared using method B, Example 2. ¹H NMR (400 MHz, DMSO-d6): δ 13.53 (s, 1H), 10.97 (s, 1H), 9.79 (s, 1H), 7.69 (d, 1H), 7.59 (m, 1H), 7.46 (s, 1H), 7.36 (m, 2H), 7.21 (m, 1H), 3.92 (s, 3H), 2.89 (s, 3H).

MS (ES+): MZ 350 (M+1).

### Example 61

### 6-Methoxy-2-(6-methoxy-4-methyl-quinazolin-2-ylamino)-quinazolin-4-ol

This compound is prepared using method B, Example 2. ¹H NMR (400 MHz, DMSO-d6): δ 13.58 (s, 1H), 11.02 (s, 1H), 7.68 (s, 1H), 7.57 (d, 1H), 7.43 (s, 2H), 7.39 (s, 1H), 7.35 (s, 1H), 3.90 (s, 3H), 3.85 (s, 3H), 2.88 (s, 3H). MS (ES+): MZ 364 (M+1).

### Example 62

### 6,7-Dimethoxy-2-(6-methoxy-4-methyl-quinazolin-2-ylamino)-quinazolin-4-ol

This compound is prepared using method B, Example 2. ¹H NMR (400 MHz, DMSO-d6): δ 13.45 (s, 1H), 11.02 (s, 1H), 7.72 (d, 1H), 7.59 (d, 1H), 7.47 (s, 1H), 7.37 (s, 1H), 6.86 (s, H), 3.93 (s, 3H), 3.90 (s, 3H), 3.85 (s, 3H), 2.89 (s, 3H). MS (ES+): MZ 394 (M+1).

### Example 63

### 2-(6-Methoxy-4-methyl-quinazolin-2-ylamino)-6-methyl-quinazolin-4-ol

This compound is prepared using method B, Example 2. ¹H NMR (400 MHz, DMSO-d6): δ 13.53 (s, 1H), 11.01 (s, 1H), 7.84 (s, 1H), 7.71 (d, 1H), 7.58 (m, 1H), 7.53 (m, 1H), 7.45 (d, 1H), 7.36 (d, 1H), 3.91 (s, 3H), 2.88 (s, 3H), 2.40 (s, 3H). MS (ES+): MZ 348 (M+1).

### Example 64

### 2-(6-Methoxy-4-methyl-quinazolin-2-ylamino)-7-methyl-quinazolin-4-ol

This compound is prepared using method B, Example 2. ¹H NMR (400 MHz, DMSO-d6): δ 7.97 (d, 1H), 7.88 (d, 1H), 7.72 (m, 1H), 7.60 (d, 1H), 7.46 (s, 1H), 7.27 (d, 1H), 3.97 (s, 3H), 3.00 (s, 3H), 2.47 (s, 3H). MS (ES+): MZ 348 (M+1).

### Example 65

### 2-(6-Methoxy-4-methyl-quinazolin-2-ylamino)-8-methyl-quinazolin-4-ol

This compound is prepared using method B, Example 2. ¹H NMR (400 MHz, DMSO-d6):δ 13.55 (s, 1H), 11.01 (s, 1H), 7.85 (d, 1H), 7.72 (m, 1H), 7.61 (m, 2H), 7.45 (s, 1H), 7.18 (t, 1H), 3.91 (s, 3H), 2.88 (s, 3H), 2.50 (s, 3H). MS (ES+): MZ 348 (M+1).

### Example 66

### 2-(6,7-Dimethoxy-4-methyl-quinazolin-2-ylamino!-8-methyl-6-nitro-quinazolin-4-ol trifluoroacetate salt

This compound is prepared using method B, Example 2. ¹H NMR (400 MHz, DMSO-d6): δ 13.93 (s, 1H), 8.59 (s, 1H), 8.32 (s, 1H), 7.40 (s, 1H), 7.14 (s, 1H), 4.00 (s, 3H), 3.93 (s, 3H), 3.25 (s, 3H), 2.86 (s, 3H), MS (ES+): MZ 423 (M+1).

### Example 67

### 2-(6,8-Dimethoxy-4-methyl-quinazolin-2-ylamino)-8-methyl-6-nitro-quinazolin-4-ol trifluoroacetate salt

This compound is prepared using method B, Example 2. ¹H NMR (400 MHz, DMSO-d6): δ 11.46 (s, 1H), 8.50 (s, 1H), 8.24 (s, 1H), 6.97 (s, 1H), 6.92 (s, 1H), 3.98 (s, 3H) 3.86 (s, 3H), 2.82 (s, 3H), MS (ES+): MZ 423 (M+1).

### Example 68

### 2-(8-Methoxy-4-methyl-quinazolin-2-ylamino)-8-methyl-6-nitro-quinazolin-4-ol trifluoroacetate salt

This compound is prepared using method B, Example 2. ¹H NMR (400 MHz, DMSO-d6): δ 8.62 (s, 1H), 8.34 (s, 1H), 7.75 (d, 1H), 7.50 (m, 2H), 4.00 (s, 3H), 2.85 (s, 3H), MS (ES+): MZ 393 (M+1).

### Example 69

### 2-[6-(2-Methoxy-ethoxy)-4-methyl-quinazolin-2-ylamino]-8-methyl-6-nitro-quinazolin-4-ol trifluoroacetate salt

This compound is prepared using method B, Example 2. ¹H NMR (400 MHz, DMSO-d6): δ 8.53 (s, 1H), 8.28 (s, 1H), 7.70 (d, 1H), 7.60 (d, 1H), 7.46 (s, 1H), 4.24 (t, 2H), 3.71 (m, 2H), 3.32 (s, 3H), 2.86 (s, 3H), MS (ES+): MZ 437 (M+1).

### Example 70

### 6-Amino-2-[6-(2-methoxy-ethoxy)-4-methyl-quinazolin-2-ylamino]-8-methyl-quinazolin-4-ol trifluoroacetate salt

This compound is prepared using method B, Example 2. ¹H NMR (400 MHz, DMSO-d6): δ 7.70 (d, 1H), 7.62 (d, 1H), 7.50 (s, 1H), 7.17 (s, 1H), 7.02 (s, 1H), 4.27 (t, 2H), 3.73 (m, 2H), 3.34 (s, 3H), 2.86 (s, 3H), MS (ES+): MZ 407 (M+1).

### Example 71

### 2-(6-Methoxy-4-methyl-quinazolin-2-ylamino)-quinazoline-4,8-diol trifluoroacetate salt

This compound is prepared using method B, Example 2. ¹H NMR (400 MHz, DMSO-d6): δ 7.74 (m, 2H), 7.56 (s, 1H), 7.49 (d, 1H), 7.22 (m, 2H), 3.96 (s, 3H), 2.96 (s, 3H), MS (ES+): MZ 350 (M+1).

### Example 72

### 2-(8-Methoxy-4-methyl-quinazolin-2-ylamino)-quinazoline-4,8-diol trifluoroacetate salt

This compound is prepared using method B, Example 2. ¹H NMR (400 MHz, DMSO-d6): δ 7.81 (d, 1H), 7.55 (m, 3H), 7.28 (m, 2H), 4.09 (s, 3H), 2.94 (s, 3H). MS (ES+): MZ 350 (M+1).

### Example 73

### 2-(7,8-Dimethoxy-4-methyl-quinazolin-2-ylamino)-6,8-dimethyl-quinazolin-4-ol trifluoroacetate salt

This compound is prepared using method B, Example 2. ¹H NMR (400 MHz, DMSO-d6): δ 8.00 (d, 1H), 7.69 (s, 1H), 7.44 (m, 2H), 4.02 (s, 3H), 3.99 (s, 3H), 2.86 (s, 3H), 2.36 (s, 3H). MS (ES+): MZ 392 (M+1).

### Example 74

### 2-(7,8-Dimethoxy-4-methyl-quinazolin-2-ylamino)-quinazolin-4-ol trifluoroacetate salt

This compound is prepared using method B, Example 2. ¹H NMR (400 MHz, DMSO-d6): δ 8.03 (m, 2H), 7.72 (t, 1H), 7.44 (m, 2H), 7.32 (t, 1H), 4.00 (s, 3H), 3.99 (s, 3H), 2.83 (s, 3H), MS (ES+): MZ 364 (M+1).

### Example 75

### 2-(6,8-Dimethoxy-4-methyl-quinazolin-2-ylamino)-6,8-dimethyl-quinazolin-4-ol trifluoroacetate salt

This compound is prepared using method B, Example 2. ¹H NMR (400 MHz, DMSO-d6): δ 7.61 (s, 1H), 7.9 (s, 1H), 7.02 (s, 1H), 6.95 (s, 1H), 3.95 (s, 1H), 3.88 (s, 3H), 2.82 (s, 3H), 2.33 (s, 3H). MS (ES+): MZ 392 (M+1).

### Example 76

### 2-(6-methoxy-7-amino-4-methyl-quinazolin-2-ylamino)-8-methoxy-quinazolin-4-ol trifluoroacetate salt

This compound is prepared using method B, Example 2. ¹H NMR (400 MHz, DMSO-d6): δ 7.66 (d, 1H), 7.58 (d, 1H), 7.45 (d, 1H), 7.35 (t, 1H), 7.00 (s, 1H), 4.06 (s, 6H), 3.89 (s, 3H), 3.15 (s, 3H). MS (ES+): MZ 379 (M+1).

### Example 77

### 2-(6-methoxy-7-nitro-4-methyl-quinazolin-2-ylamino)-8-methoxy-quinazolin-4-ol trifluoroacetate salt

This compound is prepared using method B, Example 2. ¹H NMR (400 MHz, DMSO-d6): δ 8.11 (d, 1H), 7.95 (d, 1H), 7.54 (d, 1H), 7.33 (d, 1H), 7.27 (t, 1H), 4.02 (s, 6H), 2.64 (s, 3H). MS (ES+): MZ 409 (M+1).

### Example 78

### 2-(7,8-Dimethoxy-4-methyl-quinazolin-2-ylamino)-8-methoxy-quinazolin-4-ol trifluoroacetate salt

This compound is prepared using method B, Example 2. ¹H NMR (400 MHz, DMSO-d6): δ 8.02 (d, 1H), 7.60 (m, 1H), 7.46 (m, 1H), 7.33 (m, 2H), 4.02 (s, 3H), 3.99 (s, 3H), 3.97 (s, 3H), 2.85 (s, 3H). MS (ES+): MZ 394 (M+1).

### Example 79

### 8-Methoxy-2-[6-methoxy-8-(2-methoxy-ethoxy)-4-methyl-quinazolin-2-ylamino]-quinazolin-4-ol trifluoroacetate salt

This compound is prepared using method B, Example 2. ¹H NMR (400 MHz, DMSO-d6): δ 7.58 (d, 1H), 7.28 (m, 2H), 7.01 (s, 1H), 6.91 (s, 1H), 4.29 (t, 2H), 3.90 (s, 3H), 3.85 (s, 3H), 3.72 (2H), 3.38 (s, 3H), 2.81 (s, 3H), MS (ES+): MZ 438 (M+1).

### Example 80

### 8-Methoxy-2-[6-(2-methoxy-ethoxy)-4-methyl-quinazolin-2-ylamino]-quinazolin-4-ol trifluoroacetate salt

This compound is prepared using method B, Example 2. ¹H NMR (400 MHz, DMSO-d6): δ 7.66 (m, 2H), 7.53 (d, 1H), 7.48 (s, 1H), 7.36 (d, 1H), 7.27 (d, 1H), 4.26 (t, 2H), 4.04 (s, 3H), 3.72 (s, 2H), 3.35 (s, 3H), 2.88 (s, 3H), MS (ES+): MZ 408 (M+1).

### Example 81

### 8-Methoxy-2-[7-methoxy-8-(2-methoxy-ethoxy)-4-methyl-quinazolin-2-ylamino]-quinazolin-4-ol trifluoroacetate salt

This compound is prepared using method B, Example 2. ¹H NMR (400 MHz, DMSO-d6): δ 7.58 (d, 1H), 7.27 (m, 2H), 7.01 (s, 1H), 6.92 (s, 1H), 4.28 (t, 2H), 3.90 (s, 3H), 3.85 (s, 3H), 3.72 (t, 2H), 3.38 (s, 3H), 2.81 (s, 3H), MS (ES+): MZ 438 (M+1).

### Example 82

### 8-Methoxy-2-(8-methyl-2,3-dihydro-1,4-dioxa-5,7-diaza-phenanthren-6-ylamino)-quinazolin-4-ol

This compound is prepared using method B, Example 2. ¹H NMR (400 MHz, DMSO-d6) δ 11.31 (br. s, 1H), 7.71 (m, 1H), 7.61 (m, 1H), 7.27 (m, 2H), 7.13 (m, 1H), 4.47 (m, 4H), 3.89 (s, 3H), 2.80 (s, 3H), MS (ES+): MZ 392 (M+1).

### Example 83

### 5-ethoxy-2-(8-methyl-2,3-dihydro-1,4-dioxa-5,7-diaza-phenanthren-6-ylamino)-quinazolin-4-ol trifluoroacetate salt

This compound is prepared using method B, Example 2. ¹H NMR (400 MHz, DMSO-d6) δ 7.75 (d, 1H), 7.63 (t, 1H), 7.17 (d,1H), 7.00 (d, 1H), 6.87 (d, 1H), 4.50 (s, b, 4H), 3.87 (s, 3H), 2.84 (s, 3H), MS (ES+): MZ 392 (M+1).

### Example 84

### 2-(6,7-Dimethoxy-4-methyl-quinazolin-2-ylamino)-5-methoxy-quinazolin-4-ol trifluoroacetate salt

This compound is prepared using method B, Example 2. ¹H NMR (400 MHz, DMSO-d6): δ 7.60 (t, 1H), 7.34 (s, 1H), 7.10 (s, 1H), 7.00 (d, 1H), 6.83 (d, 1H), 4.00 (s, 3H), 3.91 (s, 3H), 3.86 (s, 3H), 2.86 (s, 3H), MS (ES+): MZ 394 (M+1)

### Example 85

### 2-(6,8-Dimethoxy-4-methyl-quinazolin-2-ylamino)-5-methoxy-quinazolin-4-ol trifluoroacetate salt

This compound is prepared using method B, Example 2. 1 H NMR (400 MHz, DMSO-d6): δ 7.62 (t, 1H), 7.05 (s, 1H), 7.00 (s, 1H), 6.95 (d, 1H), 6.85 (d, 1H), 4.02 (s, 3H), 3.90 (s, 3H), 3.86 (s, 3H), 2.86 (s, 3H), MS (ES+): MZ 394 (M+1)

### Example 86

### 2-(7-Fluoro-6-methoxy-4-methyl-quinazolin-2-ylamino)-8-methoxy-quinazolin-4-ol trifluoroacetate salt

This compound is prepared using method B, Example 2. ¹H NMR (400 MHz, DMSO-d6): δ 7.64 (d, 1H), 7.53 (d, 1H), 7.46 (d, 1H), 7.34 (d, 1H), 7.28 (t, 1H), 4.01 (s, 3H), 3.97 (s, 3H), 2.89 (s, 3H), MS (ES+): MZ 382 (M+1).

### Example 87

### 2-(4-Fluoromethyl-6,7-dimethoxy-quinazolin-2-ylamino)-8-methoxy-quinazolin-4-ol trifluoroacetate salt

This compound is prepared using method B, Example 2. 1H NMR (400 MHz, DMSO-d6): δ 7.50 (d, 1H), 7.37 (d, 1H), 7.32 (s, 1H), 7.29 (m, 1H), 7.06 (s, 1H), 6.08 (s, 1H), 5.97 (s, 1H), 4.02 (s, 6H), 3.91 (s, 3H), MS (ES+): MZ 412 (M+1).

### Example 88

### 5-Methoxy-2-[6-(2-methoxy-ethoxy)-4-methyl-quinazolin-2-ylamino]-quinazolin-4-ol trifluoroacetate salt

This compound is prepared using method B, Example 2. ¹H NMR (400 MHz, DMSO-d6): δ 7.75 (d, 1H), 7.63 (m, 2H), 7.54 (s, 1H), 7.03 (d, 1H), 6.85 (d, 1H), 4.29 (t, 2H), 3.87 (s, 3H), 3.74 (b, 2H), 3.36 (s, 3H), 2.91 (s, 3H), MS (ES+): MZ 408 (M+1).

### Example 89

### 2-[6,7-Bis-(2-methoxy-ethoxy)-4-methyl-quinazolin-2-ylamino]-8-methoxy-quinazolin-4-ol trifluoroacetate salt

This compound is prepared using method B, Example 2. ¹H NMR (400 MHz, DMSO-d6): δ 6.90-7.20 (m, 5H), 4.34 (b, 2H), 4.26 (b, 2H), 4.08 (s, 3H), 3.75 (b, 4H), 3.35 (b, 6H), 2.79 (s, 3H), MS (ES+): MZ 482 (M+1).

### Example 90

### 2-(4-Fluoromethyl-6,7-dimethoxy-quinazolin-2-ylamino)-5-methoxy-quinazolin-4-ol trifluoroacetate salt

This compound is prepared using method B, Example 2. ¹H NMR (400 MHz, DMSO-d6): δ 7.61 (t, 1H), 7.35 (s, 1H), 7.18 (s, 1H), 7.00 (d, 1H), 6.86 (d, 1H), 6.09 (s, 1H), 5.98 (s, 1H), 3.99 (s, 3H), 3.90 (s, 3H), 3.84 (s, 3H), MS (ES+): MZ 412 (M+1).

### Example 91

### 8-Methoxy-2-(5-methoxy-4,8-dimethyl-quinazolin-2-ylamino)-quinazolin-4-ol trifluoroacetate salt

This compound is prepared using method B, Example 2. ¹H NMR (400 MHz, DMSO-d6): δ 7.63 (d, 1H), 7.55 (t, 1H), 6.91 (d, 1H), 6.81 (d, 1H), 6.78 (d, 1H), 3.86 (s, 3H), 3.81 (s, 3H), 2.88 (s, 3H), MS (ES+): MZ 378 (M+1).

### Example 92

### 8-Methyl-2-(5-methoxy-4,8-dimethyl-quinazolin-2-ylamino)-quinazolin-4-ol trifluoroacetate salt

This compound is prepared using method B, Example 2. ¹H NMR (400 MHz, DMSO-d6): δ 7.82 (b, 1H), 7.64 (b, 1H), 7.54 (b, 1H), 7.16 (b, 1H), 6.84 (b, 1H), 3.89 (s, 3H), 2.91 (s, 3H), MS (ES+): MZ 362 (M+1).

### Example 93

### 2-(6,8-Dimethoxy-4-methyl-quinazolin-2-ylamino)-quinazoline-4,5-diol trifluoroacetate salt

This compound is prepared using method B, Example 2. ¹H NMR (400 MHz, DMSO-d6): δ 7.60 (t, 1H), 7.10 (s, 1H), 7.04 (s, 1H), 6.81 (d, 1H), 6.68 (d, 1H), 4.08 (s, 3H), 3.93 (s, 3H), 2.87 (s, 3H), MS (ES+): MZ 380 (M+1).

### Example 94

### 2-(7-Fluoro-6-methoxy-4-methyl-quinazolin-2-ylamino)-5-methoxy-quinazolin-4-ol trifluoroacetate salt

This compound is prepared using method B, Example 2. ¹H NMR (400 MHz, DMSO-d6): δ 7.69 (d, 1H), 7.63 (m, 2H), 7.03 (d, 1H), 6.84 (d, 1H), 4.03 (s, 3H), 3.86 (s, 3H), 2.91 (s, 3H), MS (ES+): MZ 382 (M+1).

### Example 95

### 5,8-Dimethoxy-2-(8-methyl-2,3-dihydro-1,4-dioxa-5,7-diaza-phenanthren-6-ylamino)-quinazolin-4-ol trifluoroacetate salt

This compound is prepared using method B, Example 2. ¹H NMR (400 MHz, DMSO-d6): δ 7.71 (d, 1H), 7.30 (d, 1H), 7.15 (d, 1H), 6.79 (d, 1H), 4.49 (s, 4H), 3.90 (s, 3H), 3.78 (s, 3H), 2.83 (s, 3H), MS (ES+): MZ 422 (M+1).

### Example 96

### 2-(6,7-Dimethoxy-4-methyl-quinazolin-2-ylamino)-8-fluoro-quinazolin-4-ol trifluoroacetate salt

This compound is prepared using method B, Example 2. 1H NMR (400 MHz, DMSO-d6): δ 7.81 (d, 1H), 7.55 (t, 1H), 7.36 (s, 1H), 7.22 (b, 1H), 7.07 (s, 1H), 3.97 (s, 3H), 3.90 (s, 3H), 2.80 (s, 3H), MS (ES+): MZ 382 (M+1).

### Example 97

### 2-(6,7-Dimethoxy-4-methyl-quinazolin-2-ylamino)-8-ethoxy-quinazolin-4-ol trifluoroacetate salt

This compound is prepared using method B, Example 2. 1H NMR (400 MHz, DMSO-d6): δ 7.53 (d, 1H), 7.39 (s, 1H), 7.35 (d, 1H), 7.27 (t, 1H), 7.05 (s, 1H), 4.29 (q, 2H), 3.99 (s, 3H), 3.95 (s, 3H), 2.89 (s, 3H), 1.57 (t, 3H), MS (ES+): MZ 408 (M+1).

### Example 98

### 2-(6,8-Dimethoxy-4-methyl-quinazolin-2-ylamino)-8-fluoro-quinazolin-4-ol trifluoroacetate salt

This compound is prepared using method B, Example 2. 1H NMR (400 MHz, DMSO-d6): δ 7.84 (d, 1H), 7.57 (t, 1H), 7.25 (d, 1H), 7.05 (s, 1H), 7.00 (s, 1H), 4.00 (s, 3H), 3.90 (s, 3H), 2.84 (s, 3H), MS (ES+): MZ 382 (M+1).

### Example 99

### 2-(6,7-Dimethoxy-4-methyl-quinazolin-2-ylamino)-5,8-dimethoxy-quinazolin-4-ol trifluoroacetate salt

This compound is prepared using method B, Example 2. 1H NMR (400 MHz, DMSO-d6): δ 7.33 (s, 1H), 7.28 (d, 1H), 6.96 (s, 1H), 6.76 (d, 1H), 3.98 (s, 6H), 3.91 (s, 3H), 3.75 (s, 3H), 2.85 (s, 3H), MS (ES+): MZ 424 (M+1).

### Example 100

### 2-(6,8-Dimethoxy-4-methyl-quinazolin-2-ylamino)-5,8-dimethoxy-quinazolin-4-ol trifluoroacetate salt

This compound is prepared using method B, Example 2. 1H NMR (400 MHz, DMSO-d6): □ 7.34 (d, 1H), 7.05 (s, 1H), 6.98 (s, 1H), 6.83 (d, 1H), 4.01 (s, 3H), 3.94 (s, 6H), 3.82 (s, 3H), 2.87 (s, 3H), MS (ES+): MZ 424 (M+1).

### Example 101

### [2-(6,7-Dimethoxy-4-methyl-quinazolin-2-ylamino)-4-hydroxy-quinazolin-6-yloxy]-acetonitrile

This compound is prepared using method B, Example 2. 1H NMR (400 MHz, DMSO-d6): δ 13.61 (s, 1H), 10.91 (s, 1H), 7.59 (s, 1H), 7.42 (s, 2H), 7.36 (s, 1H), 7.07 (s, 1H), 5.27 (s, 2H), 3.99 (s, 3H), 3.91 (s, 3H), 2.81 (s, 3H), MS (ES+): MZ 419 (M+1).

### Example 102

### 2-(6,7-Dimethoxy-4-methyl-quinazolin-2-ylamino)-6-(2-methoxy-ethoxy)-quinazolin-4-ol trifluoroacetate salt

This compound is prepared using method B, Example 2. 1H NMR (400 MHz, DMSO-d6): δ 7.48 (d, 1H), 7.36 (d, 1H), 7.34 (s, 1H), 7.30 (s, 1H), 7.11 (s, 1H), 4.14 (t, 2H), 3.98 (s, 3H), 3.88 (s, 3H), 3.67 (t, 2H), 3.31 (s, 3H), 2.83 (s, 3H), MS (ES+): MZ 438 (M+1).

### Example 103

### 2-(4-Hydroxy-8-methoxy-quinazolin-2-ylamino)-6-methoxy-4-methyl-quinazolin-7-ol trifluoroacetate salt

This compound is prepared using method B, Example 2. 1H NMR (400 MHz, DMSO-d6): δ 7.51 (d, 1H), 7.40 (d, 1H), 7.38 (s, 1H), 7.28 (t, 1H), 7.05 (s, 1H), 4.08 (s, 3H), 3.93 (s, 3H), 2.87 (s, 3H), MS (ES+): MZ 380 (M+1).

### Example 104

### 2-(8-Fluoro-4-hydroxy-quinazolin-2-ylamino)-6-methoxy-4-methyl-quinazolin-7-ol trifluoroacetate salt

This compound is prepared using method B, Example 2. 1H NMR (400 MHz, DMSO-d6): δ 7.85 (d, 1H), 7.60 (t, 1H), 7.41 (s, 1H), 7.26 (b, 1H), 7.06 (s, 1H), 3.95 (s, 3H), 2.83 (s, 3H), MS (ES+): MZ 368 (M+1).

### Example 105

### 6-(2-Chloro-ethoxy)-2-(6,7-dimethoxy-4-methyl-quinazolin-2-ylamino)-quinazolin-4-ol trifluoroacetate salt

This compound is prepared using method B, Example 2. 1H NMR (400 MHz, DMSO-d6): δ 7.43 (d, 1H), 7.35 (s, 1H), 7.32 (d, 1H), 7.30 (s, 1H), 7.07 (s, 1H), 4.29 (t, 2H), 3.95 (s, 3H), 3.92 (t, 2H), 3.85 (s, 3H), 2.79 (s, 3H), MS (ES+): MZ 442 (M+1).

### Example 106

### 6-(3-Chloro-propoxy)-2-(6,7-dimethoxy-4-methyl-quinazolin-2-ylamino(-quinazolin-4-ol trifluoroacetate salt

This compound is prepared using method B, Example 2. 1H NMR (400 MHz, DMSO-d6): δ 7.46 (d, 1H), 7.45 (s, 1H), 7.42 (s, 1H), 7.39 (m, 1H), 7.17 (s, 1H), 4.17 (t, 2H), 4.01 (s, 3H), 3.94 (s, 3H), 3.82 (t, 2H), 2.86 (s, 3H), 2.20 (m, 2H) MS (ES+): MZ 456 (M+1).

### Example 107

### 6-(2-Chloro-ethoxy)-2-(6,8-dimethoxy-4-methyl-quinazolin-2-ylamino)-quinazolin-4-ol trifluoroacetate salt

This compound is prepared using method B, Example 2. 1H NMR (400 MHz, DMSO-d6): δ 7.44 (s, 1H), 7.38 (s, 2H), 7.03 (s, 1H), 6.98 (s, 1H), 4.33 (t, 2H), 4.00 (s, 3H), 3.97 (m, 2H), 3.88 (s, 3H), 2.83 (s, 3H), MS (ES+): MZ 442 (M+1).

### Example 108

### 8-Fluoro-2-(4-methyl-7,8-dihydro-[1,4]dioxino[2,3-g]quinazolin-2-ylamino)-quinazolin-4-ol trifluoroacetate salt

This compound is prepared using method B, Example 2. 1H NMR (400 MHz, DMSO-d6): δ 7.84 (d, 1H), 7.57 (s, 1H), 7.54 (d, 1H), 7.24 (m, 1H), 7.10 (s, 1H), 4.43 (s, 2H), 4.36 (s, 2H), 2.76 (s, 3H), MS (ES+): MZ 380 (M+1).

### Example 109

### 7-(6,7-Dimethoxy-4-methyl-quinazolin-2-ylamino)-1,3-dioxa-6,8-diaza-cyclopenta[a]naphthalen-9-ol trifluoroacetate salt

This compound is prepared using method B, Example 2. 1H NMR (400 MHz, DMSO-d6): δ 7.36 (s, 1H), 7.31 (d, 1H), 7.12 (s, 1H), 6.95 (d, 1H), 6.22 (d, 2H), 4.01 (s, 3H), 3.92 (s, 3H), 2.86 (s, 3H), MS (ES+): MZ 408 (M+1).

### Example 110

### 2-(6,7-Dimethoxy-4-methyl-quinazolin-2-ylamino)-5-isopropoxy-quinazolin-4-ol acetate salt

This compound is prepared using method B, Example 2. 1H NMR (400 MHz, DMSO-d6): δ 7.59 (t, 1H), 7.38 (s, 1H), 7.14 (s, 1H), 7.03 (d, 1H), 6.67 (d, 1H), 4.69 (m, 1H), 4.03 (s, 3H), 3.92 (s, 3H), 2.87 (s, 3H), 1.33 (d, 6H). MS (ES+): MZ 422 (M+1).

### Example 111

### 2-(6,8-Dimethoxy-4-methyl-quinazolin-2-ylamino)-5-isopropoxy-quinazolin-4-ol trifluoroacetate salt

This compound is prepared using method B, Example 2. 1H NMR (400 MHz, DMSO-d6): δ 7.57 (t, 1H), 7.08 (s, 1H), 7.02 (s, 1H), 6.90 (d, 1H), 6.84 (d, 1H), 4.69 (m, 1H), 4.10 (s, 3H), 3.92 (s, 3H), 2.86 (s, 3H), 1.33 (d, 6H). MS (ES+): MZ 422 (M+1).

### Example 112

### 2-(6,8-Dimethoxy-4-methyl-quinazolin-2-ylamino)-5-ethoxy-quinazolin-4-ol trifluoroacetate salt

This compound is prepared using method B, Example 2. 1H NMR (400 MHz, DMSO-d6): δ 7.59 (t, 1H), 7.09 (s, 1H), 7.03 (s, 1H), 6.95 (d, 1H), 6.82 (d, 1H), 4.12 (m, 2H), 3H), 3.92 (s, 3H), 2.86 (s, 3H), 1.39 (t, 3H). MS (ES+): MZ 408 (M+1).

### Example 113

### 2-(6,7-Dimethoxy-4-methyl-quinazolin-2-ylamino)-6-piperidin-1-yl-quinazolin-4-ol trifluoroacetate salt

This compound is prepared using method B, Example 2. 1H NMR (400 MHz, DMSO-d6): δ 7.64 (d, 2H), 7.52 (d, 1H), 7.36 (s, 1H), 7.17 (s, 1H), 3.99 (s, 3H), 3.90 (s, 3H), 3.32 (b, 4H), 2.88 (s, 3H), 1.71 (b, 4H), 1.59 (b, 2H), MS (ES+): MZ 447 (M+1),

### Example 114

### 2-(6,7-Dimethoxy-4-methyl-quinazolin-2-ylamino)-6-morpholin-4-yl-quinazolin-4-ol trifluoroacetate salt

This compound is prepared using method B, Example 2. 1H NMR (400 MHz, DMSO-d6): δ 7.50 (d, 2H), 7.40 (s, 1H), 7.36 (s, 1H), 7.17 (s, 1H), 4.00 (s, 3H), 3.93 (s, 3H), 3.75 (t, 4H), 3.16 (t, 4H), 2.86 (s, 3H), MS (ES+): MZ 449 (M+1).

### Example 115

### 2-[6-(2-Diethylamino-ethoxy)-4-methyl-quinazolin-2-ylamino]-8-methoxy-quinazolin-4-ol trifluoroacetate salt

This compound is prepared using method B, Example 2. 1H NMR (400 MHz, DMSO-d6) δ 7.62 (m, 1H), 7.55 (m, 1H), 7.52 (m, 1H), 7.45 (m, 1H), 7.35 (m, 1H), 7.27 (m, 1H), 4.22 (m, 2H), 4.13 (s, 3H), 2.98 (m, 6H), 2.87 (s, 3H), 1.16 (t, 6H) MS (ES+): MZ 449 (M+1).

### Example 116

### 8-Methoxy-2-(6,7,8-trimethoxy-4-methyl-quinazolin-2-ylamino)-quinazolin-4-ol trifluoroacetate salt

This compound is prepared using method B, Example 2. 1H NMR (400 MHz, DMSO-d6): δ 8.22 (b s, 1H), 7.57 (m, 1H), 7.48-7.25 (m, 3H), 4.02 (s, 3H), 4.00 (s, 3H), 3.97 (s, 3H), 3.96 (s, 3H), 2.85 (s, 3H). MS (ES+): MZ 424 (M+1).

### Example 117

### 2-(7-Fluoro-8-methoxy-4-methyl-quinazolin-2-ylamino)-8-methoxy-quinazolin-4-ol trifluoroacetate salt

This compound is prepared using method B, Example 2. 1H NMR (400 MHz, DMSO-d6): δ 8.02 (d, 1H), 7.60 (m, 1H), 7.56-7.45 (m, 1H), 7.35-7.25 (m, 2H), 4.10 (s, 3H), 3.95 (s, 3H), 2.87 (s, 3H). MS (ES+): MZ 382 (M+1).

### Example 118

### 8-Fluoro-2-(6,7,8-trimethoxy-4-methyl-quinazolin-2-ylamino)-quinazolin-4-ol trifluoroacetate salt

This compound is prepared using method B, Example 2. 1H NMR (400 MHz, DMSO-d6): δ 7.85 (d, 1H), 7.57 (m, 1H), 7.30 (s, 1H), 7.29-7.22 (m, 1H), 4.06 (s, 3H), 3.98 (s, 3H), 3.95 (s, 3H), 2.83 (s, 3H). MS (ES+): MZ 412 (M+1).

### Example 119

### 8-Fluoro-2-(7-fluoro-8-methoxy-4-methyl-quinazolin-2-ylamino)-quinazolin-4-ol trifluoroacetate salt

This compound is prepared using method B, Example 2. 1H NMR (400 MHz, DMSO-d6): δ 8.02 (d, 1H), 7.86 (d, 1H), 7.62-7.57 (m, 1H), 7.53-7.47 (m, 1H), 7.32-7.52 (m, 1H), 4.13 (s, 3H), 2.86 (s, 3H). MS (ES+): MZ 370 (M+1).

### Example 120

### 6-Amino-2-(6,7-dimethoxy-4-methyl-quinazolin-2-ylamino)-8-methyl-quinazolin-4-ol trifluoroacetate salt

This compound is prepared using method B, Example 2. 1H NMR (400 MHz, DMSO-d6): δ 7.42 (s, 1H), 7.32 (s, 1H), 7.15 (s, 1H), 7.10 (s, 1H), 4.00 (s, 3H), 3.95 (s, 3H), 2.87 (s, 3H), MS (ES+): MZ 393 (M+1).

### Example 121

### 2-(6,7-Dimethoxy-4-methyl-quinazolin-2-ylamino)-6-dimethylamino-8-methyl-quinazolin-4-ol trifluoroacetate salt

This compound is prepared using method B, Example 2. 1H NMR (400 MHz, DMSO-d6): δ 7.23 (s, 1H), 7.08 (s, 1H), 6.88 (s, 1H), 6.82 (s, 1H), 3.91 (s, 3H), 3.88 (s, 3H), 2.89 (s, 6H), 2.79 (s, 3H), 2.42 (s, 3H), MS (ES+): MZ 421 (M+1).

### Example 122

### 8-Amino-2-(6-methoxy-4-methyl-quinazolin-2-ylamino)-quinazolin-4-ol trifluoroacetate salt

This compound is prepared using method B, Example 2. 1H NMR (400 MHz, DMSO-d6): δ 7.74 (d, 1H), 7.62 (d, 1H), 7.51 (s, 1H), 7.35 (d, 1H), 7.06 (m, 2H), 3.95 (s, 3H), 2.95 (s, 3H), MS (ES+): MZ 349 (M+1).

### Example 123

### 6-Amino-2-(6-methoxy-4-methyl-quinazolin-2-ylamino)-8-methoxy-quinazolin-4-ol trifluoroacetate salt

This compound is prepared using method B, Example 2. 1H NMR (400 MHz, DMSO-d6): δ 7.70 (d, 1H), 7.65 (d, 1H), 7.50 (s, 1H), 6.79 (s, 1H), 6.76 (s, 1H), 3.99 (s, 3H), 3.92 (s, 3H), 2.91 (s, 3H), MS (ES+): MZ 379 (M+1).

### Example 124

### 6-Amino-2-(6,8-dimethoxy-4-methyl-quinazolin-2-ylamino)-8-methoxy-quinazolin-4-ol trifluoroacetate salt

This compound is prepared using method B, Example 2. 1H NMR (400 MHz, DMSO-d6): δ 7.08 (s, 1H), 7.03 (s, 1H), 6.86 (s, 1H), 6.79 (s, 1H), 3.99 (s, 3H), 3.92 (s, 6H), 2.87 (s, 3H), MS (ES+): MZ 409 (M+1).

### Example 125

### 6-Amino-2-[6-(2-diethylamino-ethoxy)-4-methyl-quinazolin-2-ylamino]-8-methyl-quinazolin-4-ol trifluoroacetate salt

This compound is prepared using method B, Example 2. 1 H NMR (400 MHz, DMSO-d6): δ 7.76 (d, 1H), 7.59 (d, 1H), 7.54 (s, 1H), 7.18 (s, 1H), 7.03 (s, 1H), 4.49 (t, 2H), 3.59 (s, 2H), 3.26 (m, 4H), 2.87 (s, 3H), 1.25 (t, 6H), MS (ES+): MZ 448 (M+1).

### Example 126

### 6-Amino-2-(6,8-dimethoxy-4-methyl-quinazolin-2-ylamino)-8-methyl-quinazolin-4-ol trifluoroacetate salt

This compound is prepared using method B, Example 2. 1H NMR (400 MHz, DMSO-d6): δ 7.38 (s, 1H), 7.18 (s, 1H), 7.08 (s, 1H), 7.02 (s, 1H), 3.99 (s, 3H), 3.92 (s, 3H), 2.87 (s, 3H), MS (ES+): MZ 393 (M+1).

### Example 127

### 2-(7-Dimethylamino-6-methoxy-4-methyl-quinazolin-2-ylamino)-8-methoxy-quinazolin-4-ol trifluoroacetate salt

This compound is prepared using method B, Example 2. 1H NMR (400 MHz, DMSO-d6): δ 7.51 (d, 1H), 7.42 (d, 1H), 7.30 (t, 1H), 7.25 (s, 1H), 6.74 (s, 1H), 4.07 (s, 3H), 3.95 (s, 3H), 3.12 (s, 6H), 2.86 (s, 3H), MS (ES+): MZ 407 (M+1).

### Example 128

### 2-(6,7-Dimethoxy-4-methyl-quinazolin-2-ylamino)-5-dimethylamino-quinazolin-4-ol trifluoroacetate salt

This compound is prepared using method B, Example 2. 1H NMR (400 MHz, DMSO-d6): δ 7.94 (t, 1H), 7.83 (b, 2H), 7.43 (s, 1H), 7.36 (b, 1H), 4.01 (s, 3H), 3.94 (s, 3H), 3.28 (s, 6H), 2.88 (s, 3H), MS (ES+): MZ 407 (M+1).

### Example 129

### 2-(6,7-Dimethoxy-4-methyl-quinazolin-2-ylamino)-6-(2H-tetrazol-5-ylmethoxy)-quinazolin-4-ol trifluoroacetate salt

This compound is prepared using method B, Example 2. 1H NMR (400 MHz, DMSO-d6): δ 7.61 (s, 1H), 7.46 (s, 2H), 7.41 (s, 1H), 7.14 (s, 1H), 5.58 (s, 2H), 4.00 (s, 3H), 3.93 (s, 3H), 2.83 (s, 3H), MS (ES+): MZ 462 (M+1).

### Example 130

### 7-(6,8-Dimethoxy-4-methyl-quinazolin-2-ylamino)-1,3-dioxa-6,8-diaza-cyclopenta[a]naphthalen-9-ol trifluoroacetate salt

This compound is prepared using method B, Example 2. ¹H NMR (400 MHz, DMSO-d6): δ 7.31 (d, 1H), 7.02 (s, 1H), 6.96 (s, 1H), 6.85 (d, 1H), 6.21 (s, 2H), 4.01 (s, 3H), 3.89 (s, 3H), 2.83 (s, 3H), MS (ES+): MZ 408 (M+1).

### Example 131

### 7-(6,8-Dimethoxy-4-methyl-quinazolin-2-ylamino)-1,3-dioxa-6.8-diaza-cyclopenta[a]naphthalen-9-ol HCl salt

This compound is prepared using method B, Example 2. ¹H NMR (400 MHz, DMSO-d6): δ 7.44 (d, 1H), 7.16 (s, 1H), 7.09 (s, 1H), 6.90 (d, 1H), 6.31 (s, 2H), 4.10 (s, 3H), 3.95 (s, 3H), 2.92 (s, 3H), MS (ES+): MZ 408 (M+1).

### Example 132

### 2-(6,7-Dimethoxy-4-methyl-quinazolin-2-ylamino)-5,6-dimethoxy-quinazolin-4-ol TFA salt

This compound is prepared using method B, Example 2. NMR (400 MHz, DMSO-d6): δ 7.52 (d, 1H), 7.39 (s, 1H), 7.25 (d, 1H), 7.13 (s, 1H), 3.99 (s, 3H), 3.92 (s, 3H), 3.84 (s, 3H), 3.77 (s, 3H), 2.85 (s, 3H). MS (ES+): MZ 424 (M+1).

### Example 133

### 2-(6,7-Dimethoxy-4-methyl-quinazolin-2-ylamino)-5,6-dimethoxy-quinazolin-4-ol HCl salt

This compound is prepared using method B, Example 2. NMR (400 MHz, DMSO-d6): δ 7.56 (d, 1H), 7.41 (s, 1H), 7.38 (d, 1H), 7.27 (s, 1H), 3.99 (s, 3H), 3.92 (s, 3H), 3.85 (s, 3H), 3.78 (s, 3H), 2.89 (s, 3H). MS (ES+): MZ 424 (M+1).

### Example 134

### 2-(6,8-Dimethoxy-4-methyl-quinazolin-2-ylamino)-5,6-dimethoxy-quinazolin-4-ol TFA salt

This compound is prepared using method B, Example 2. NMR (400 MHz, DMSO-d6): δ 7.51 (d, 1H), 7.15 (d, 1H), 7.03 (s, 1H), 6.96 (s, 1H), 4.00 (s, 3H), 3.87 (s, 3H), 3.83 (s, 3H), 3.78 (s, 3H), 2.83 (s, 3H). MS (ES+): MZ 424 (M+1).

### Example 135

### 2-(6,8-Dimethoxy-4-methyl-quinazolin-2-ylamino)-5,6-dimethoxy-quinazolin-4-ol HCl salt

This compound is prepared using method B, Example 2. NMR (400 MHz, DMSO-d6): δ 7.58 (d, 1H), 7.16 (d, 1H), 7.06 (s, 1H), 6.98 (s, 1H), 4.04 (s, 3H), 3.88 (s, 3H), 3.85 (s, 3H), 3.79 (s, 3H), 2.85 (s, 3H). MS (ES+): MZ 424 (M+1).

### Example 136

### 2-(6,7-Dimethoxy-4-methyl-quinazolin-2-ylamino)-6-(2-hydroxy-ethoxy)-quinazolin-4-ol trifluoroacetate salt

This compound is prepared using method C, Example 3. ¹H NMR (400 MHz, DMSO-d6): δ 7.38-7.45 (m, 4H), 7.11 (s, 1H), 4.07 (t, 2H), 4.01 (s, 3H), 3.95 (s, 3H), 3.75 (t, 2H), 2.84 (s, 3H). MS (ES+): MZ 424 (M+1).

### Example 137

### 2-(6,8-Dimethoxy-4-methyl-quinazolin-2-ylamino)-6-(2-hydroxy-ethoxy)-quinazolin-4-ol trifluoroacetate salt

This compound is prepared using method C, Example 3. ¹H NMR (400 MHz, DMSO-d6): δ 7.44 (s, 1H), 7.35-7.44 (m, 2H), 7.05 (s, 1H), 7.00 (s, 1H), 4.05 (t, 2H), 4.00 (s, 3H), 3.90 (s, 3H), 3.75 (t, 2H), 2.84 (s, 3H). MS (ES+): MZ 424 (M+1).

### Example 138

### 6-[2-(3-Hydroxy-pyrrolidin-1-yl)-ethoxy]-2-(8-methoxy-4-methyl-quinazolin-2-ylamino)-quinazolin-4-ol trifluoroacetate salt

This compound is prepared using method C, Example 3. ¹H NMR (400 MHz, DMSO-d6): δ 7.73 (q, 1H), 7.39-7.55 (m, 5H), 4.40 (t, 2H), 4.02 (s, 3H), 3.69 (m, 1H), 3.62 (t, 2H), 3.14-3.40 (m, 6H), 2.86 (s, 3H). MS (ES+): 463 (M+1).

### Example 139

### 2-(6,7-Dimethoxy-4-methyl-quinazolin-2-ylamino)-6-{2-[(2-hydroxy-ethyl)-methyl-amino]-ethoxy}-quinazolin-4-ol trifluoroacetate salt

This compound is prepared using method C, Example 3. ¹H NMR (400 MHz, DMSO-d6): δ 7.31-7.44 (m, 4H), 7.09 (s, 1H), 4.14 (t, 2H), 3.99 (s, 3H), 3.93 (s, 3H), 3.60 (t, 2H), 3.49 (t, 2H), 2.93 (t, 2H), 2.82 (s, 3H), 2.53 (s, 3H). MS (ES+): MZ 481 (M+1).

### Example 140

### 2-(6,8-Dimethoxy-4-methyl-quinazolin-2-ylamino)-6-{2-[(2-hydroxy-ethyl)-methyl-amino]-ethoxy}-quinazolin-4-ol trifluoroacetate salt

This compound is prepared using method C, Example 3. ¹H NMR (400 MHz, DMSO-d6): δ 7.46 (s, 1H), 7.35-7.38 (m, 2H), 7.05 (s, 1H), 7.00 (s, 1H), 4.19 (t, 2H), 3.98 (s, 3H), 3.90 (s, 3H), 3.59 (m, 4H), 2.93 (t, 2H), 2.83 (s, 3H), 2.53 (s, 3H). MS (ES+): MZ 481 (M+1).

### Example 141

### 2-(6,8-Dimethoxy-4-methyl-quinazolin-2-ylamino)-6-(2-morpholin-4-yl-ethoxy)-quinazolin-4-ol trifluoroacetate salt

This compound is prepared using method C, Example 3. ¹H NMR (400 MHz, DMSO-d6): δ 7.45 (s, 1H), 7.35-7.36 (m, 2H), 7.03 (s, 1H), 6.99 (s, 1H), 4.16 (t, 2H), 3.98 (s, 3H), 3.89 (s, 3H), 3.74 (t, 4H), 3.58 (t, 2H), 3.06 (t, 4H), 2.82 (s, 3H). MS (ES+): MZ 493 (M+1).

### Example 142

### 2-(6,7-Dimethoxy-4-methyl-quinazolin-2-ylamino)-6-[2-(1-hydroxymethyl-2-methyl-propylamino)-ethoxy]-quinazolin-4-ol Trifluoroacetate salt

This compound is prepared using method C, Example 3. ¹H NMR (400 MHz, DMSO-d6): δ 7.46 (s, 1H), 7.39 (s, 1H), 7.36 (d, 1H), 7.34 (d, 1H), 7.09 (s, 1H), 4.19 (m, 2H), 4.00 (s, 3H), 3.93 (s, 3H), 3.61 (t, 2H), 3.44 (m, 2H), 3.13 (m, 1H), 1.86 (m, 1H), 0.89 (q, 6H). MS (ES+): MZ 509 (M+1).

### Example 143

### 2-(6,8-Dimethoxy-4-methyl-quinazolin-2-ylamino)-6-[2-(2-hydroxy-1-methyl-ethylamino)-ethoxy]-quinazolin-4-ol trifluoroacetate salt

This compound is prepared using method C, Example 3. ¹H NMR (400 MHz, DMSO-d6): δ 7.46 (s, 1H), 7.39 (s, 1H), 7.36 (d, 1H), 7.34 (d, 1H), 7.09 (s, 1H), 4.19 (m, 2H), 4.00 (s, 3H), 3.93 (s, 3H), 3.49 (m, 2H), 3.14 (m, 2H), 2.89 (m, 1H), 2.82 (s, 3H), 1.90 (d, 3H), MS (ES+): MZ 481 (M+1).

### Example 144

### 6-(2-Diethylamino-ethoxy)-2-(6,8-dimethoxy-4-methyl-quinazolin-2-ylamino)-quinazolin-4-ol trifluoroacetate salt

This compound is prepared using method C, Example 3. ¹H NMR (400 MHz, DMSO-d6): δ 7.56 (s, 1H), 7.43 (m, 2H), 7.08 (s, 1H), 7.03 (s, 1H), 4.43 (t, 2H), 4.02 (s, 3H), 3.97 (s, 3H), 3.59 (m, 2H), 3.26 (m, 4H), 2.89 (s, 3H), 1.25 (t, 6H), MS (ES+): MZ 479 (M+1).

### Example 145

### 2-(6,7-Dimethoxy-4-methyl-quinazolin-2-ylamino)-6-(2-piperidin-1-yl-ethoxy)-quinazolin-4-ol trifluoroacetate salt

This compound is prepared using method C, Example 3. ¹H NMR (400 MHz, DMSO-d6): δ 7.56 (s, 1H), 7.48 (d, 1H), 7.44 (m, 2H), 7.16 (s, 1H), 4.46 (t, 2H), 4.02 (s, 3H), 3.95 (s, 3H), 3.61 (t, 2H), 3.54 (m, 2H), 3.02 (m, 2H), 2.86 (s, 3H), 1.60-1.90 (m, 6H), MS (ES+): MZ 491 (M+1).

### Example 146

### 2-(6,7-Dimethoxy-4-methyl-quinazolin-2-ylamino)-6-(2-dimethylamino-ethoxy)-quinazolin-4-ol trifluoroacetate salt

This compound is prepared using method C, Example 3. ¹H NMR (400 MHz, DMSO-d6): δ 7.56 (s, 1H), 7.49 (d, 1H), 7.44 (m, 2H), 7.16 (s, 1H), 4.44 (t, 2H), 4.02 (s, 3H), 3.95 (s, 3H), 3.56 (m, 2H), 2.89 (s, 6H), 2.76 (s, 3H), MS (ES+): MZ 451 (M+1).

### Example 147

### 2-(6,7-Dimethoxy-4-methyl-quinazolin-2-ylamino)-6-[2-(4-methyl-piperazin-1-yl)-ethoxy]-quinazolin-4-ol trifluoroacetate salt

This compound is prepared using method C, Example 3. ¹H NMR (400 MHz, DMSO-d6): δ 7.47 (s, 1H), 7.46 (d, 1H), 7.39 (s, 1H), 7.38 (d, 1H), 7.14 (s, 1H), 4.28 (t, 2H), 3.99 (s, 3H), 3.91 (s, 3H), 3.00-3.60 (m, 10H), 2.84 (s, 3H), 2.80 (s, 3H), MS (ES+): MZ 506 (M+1).

### Example 148

### 2-(6,7-Dimethoxy-4-methyl-quinazolin-2-ylamino)-6-(2-pyrrolidin-1-yl-ethoxy)-quinazolin-4-ol trifluoroacetate salt

This compound is prepared using method C, Example 3. ¹H NMR (400 MHz, DMSO-d6): δ 7.56 (s, 1H), 7.49 (d, 1H), 7.44 (m, 2H), 7.16 (s, 1H), 4.42 (t, 2H), 4.02 (s, 3H), 3.95 (s, 3H), 3.63 (m, 4H), 3.18 (m, 2H), 2.86 (s, 3H), 2.05 (m, 2H), 1.90 (m, 2H), MS (ES+): MZ 477 (M+1).

### Example 149

### 6-(2-Diethylamino-ethoxy)-2-(6,7-dimethoxy-4-methyl-quinazolin-2-ylamino)-quinazolin-4-ol trifluoroacetate salt

This compound is prepared using method C, Example 3. ¹H NMR (400 MHz, DMSO-d6): δ 7.56 (s, 1H), 7.51 (d, 1H), 7.43 (s, 1H), 7.40 (d, 1H), 7.16 (s, 1H), 4.44 (t, 2H), 4.02 (s, 3H), 3.95 (s, 3H), 3.56 (m, 2H), 3.26 (m, 4H), 2.89 (m, 1H), 1.25 (t, 6H), MS (ES+): MZ 479 (M+1).

### Example 150

### 2-[4-Hydroxy-6-(2-morpholin-4-yl-ethoxy)-quinazolin-2-ylamino]-6,7-dimethoxy-quinazolin-4-ol trifluoroacetate salt

This compound is prepared using method C, Example 3. ¹H NMR (400 MHz, DMSO-d6): δ 7.56 (s, 1H), 7.49 (m, 2H), 7.42 (m, 2H), 7.14 (s, 1H), 4.46 (t, 2H), 4.01 (s, 3H), 3.94 (s, 3H), 3.20-4.00 (b, 10H), 2.86 (s, 3H), MS (ES+): MZ 493 (M+1).

### Example 151

### 2-(6,7-Dimethoxy-4-methyl-quinazolin-2-ylamino)-6-(3-pyrrolidin-1-yl-propoxy)-quinazolin-4-ol trifluoroacetate salt

This compound is prepared using method C, Example 3. ¹H NMR (400 MHz, DMSO-d6): δ 7.52 (d, 1H), 7.47 (s, 1H), 7.43 (s, 1H), 7.37 (d, 1H), 7.20 (s, 1H), 4.16 (t, 2H), 4.01 (s, 3H), 3.94 (s, 3H), 3.29 (m, 4H), 3.02 (m, 2H), 2.86 (s, 3H), 2.14 (m, 2H), 2.01 (m, 2H), 1.87 (m, 2H), MS (ES+): MZ 491 (M+1).

### Example 152

### 2-(6,7-Dimethoxy-4-methyl-quinazolin-2-ylamino)-6-(2-pyrrol-1-yl-ethoxy)-quinazolin-4-ol trifluoroacetate salt

This compound is prepared using method C, Example 3. ¹H NMR (400 MHz, DMSO-d6): δ 7.49 (d, 1H), 7.41 (m, 2H), 7.35 (m, 1H), 7.16 (s, 1H), 6.84 (d, 2H), 5.98 (t, 2H), 4.28 (t, 2H), 4.00 (s, 3H), 3.92 (s, 3H), 3.58 (m, 2H), 2.85 (s, 3H), MS (ES+): MZ 473 (M+1).

### Example 153

### 2-(6,7-Dimethoxy-4-methyl-quinazolin-2-ylamino)-6-(2-pyrrolidin-1-yl-ethoxy)-quinazolin-4-ol trifluoroacetate salt

This compound is prepared using method C, Example 3. ¹H NMR (400 MHz, DMSO-d6): δ 9.84 (s, 1H), 7.55 (s, 1H), 7.53 (d, 1H), 7.45 (d, 1H), 7.42 (s, 1H), 7.17 (s, 1H), 4.42 (t, 2H), 4.02 (s, 3H), 3.95 (s, 3H), 3.64 (m, 4H), 3.17 (m, 2H), 2.85 (s, 3H), 2.05 (m, 2H), 1.90 (m, 2H), MS (ES+): MZ 477 (M+1).

### Example 154

### 2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt

This compound is prepared using method C, Example 3. ¹H NMR (400 MHz, DMSO-d6): δ 7.52 (s, 1H), 7.51 (d, 1H), 7.42 (d, 1H), 7.39 (s, 1H), 7.15 (s, 1H), 4.46 (d, 1H), 4.43 (t, 2H), 4.02 (s, 3H), 3.93 (s, 3H), 3.10-3.80 (m, 6H), 2.85 (s, 3H), 1.90-2.40 (m, 3H), MS (ES+): MZ 493 (M+1).

### Example 155

### 2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt

This compound is prepared using method C, Example 3. ¹H NMR (400 MHz, DMSO-d6): δ 7.52 (s, 1H), 7.51 (d, 1H), 7.42 (d, 1H), 7.39 (s, 1H), 7.15 (s, 1H), 4.46 (d, 1H), 4.43 (t, 2H), 4.02 (s, 3H), 3.93 (s, 3H), 3.10-3.80 (m, 6H), 2.85 (s, 3H), 1.90-2.40 (m, 3H), MS (ES+): MZ 493 (M+1).

### Example 156

### 2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt

This compound is prepared using method C, Example 3. ¹H NMR (400 MHz, DMSO-d6): δ 7.52 (s, 1H), 7.51 (d, 1H), 7.42 (d, 1H), 7.39 (s, 1H), 7.15 (s, 1H), 4.46 (d, 1H), 4.43 (t, 2H), 4.02 (s, 3H), 3.93 (s, 3H), 3.10-3.80 (m, 6H), 2.85 (s, 3H), 1.90-2.40 (m, 2H), MS (ES+): MZ 493 (M+1).

### Example 157

### 2-(6,7-Dimethoxy-4-methyl-quinazolin-2-ylamino)-6-[2-(3-hydroxy-piperidin-1-yl)-ethoxy]-quinazolin-4-ol trifluoroacetate salt

This compound is prepared using method C, Example 3. ¹H NMR (400 MHz, DMSO-d6): δ 7.56 (d, 1H), 7.51 (d, 1H), 7.43 (s, 2H), 7.17 (s, 1H), 4.47 (t, 2H), 4.02 (s, 3H), 3.95 (s, 3H), 3.58 (b, 6H), 2.86 (s, 3H), 1.00-2.20 (m, 5H), MS (ES+): MZ 507 (M+1).

### Example 158

### 6-{2-[Bis-(2-hydroxy-ethyl)-amino]-ethoxy}-2-(6,7-dimethoxy-4-methyl-quinazolin-2-ylamino)-quinazolin-4-ol trifluoroacetate salt

This compound is prepared using method C, Example 3. ¹H NMR (400 MHz, DMSO-d6): δ 7.60 (d, 1H), 7.55 (d, 1H), 7.46 (m, 2H), 7.27 (s, 1H), 4.52 (t, 2H), 4.03 (s, 3H), 3.96 (s, 3H), 3.83 (m, 6H), 3.40 (m, 4H), 2.90 (s, 3H), MS (ES+): MZ 511 (M+1).

### Example 159

### 2-(6,7-Dimethoxy-4-methyl-quinazolin-2-ylamino)-6-{2-[ethyl-(2-hydroxy-ethyl)-amino]-ethoxy}-quinazolin-4-ol trifluoroacetate salt

This compound is prepared using method C, Example 3. ¹H NMR (400 MHz, DMSO-d6): δ 9.62 (s, 1H), 7.51 (m, 2H), 7.40 (m, 2H), 7.13 (s, 1H), 4.45 (t, 2H), 4.00 (s, 3H), 3.96 (s, 3H), 3.79 (m, 2H), 3.61 (m, 2H), 3.31 (m, 4H), 2.83 (s, 3H), 1.28 (t, 3H). MS (ES+): MZ 495 (M+1).

### Example 160

### 2-(6,8-Dimethoxy-4-methyl-quinazolin-2-ylamino)-6-[2-(3-hydroxy-pyrrolidin-1-yl)-ethoxy]-quinazolin-4-ol trifluoroacetate salt

This compound is prepared using method C, Example 3. ¹H NMR (400 MHz, DMSO-d6): δ 7.52 (s, 1H), 7.51 (s, 2H), 7.42 (s, 1H), 7.39 (s, 1H), 4.48 (d, 1H), 4.43 (t, 2H), 4.02 (s, 3H), 3.97 (s, 3H), 3.10-3.80 (m, 6H), 2.85 (s, 3H), 1.90-2.40 (m, 3H), MS (ES+): MZ 493 (M+1).

### Example 161

### 2-(6,7-Dimethoxy-4-methyl-quinazolin-2-ylamino)-6-[2-(2-hydroxy-ethylamino)-ethoxy]-quinazolin-4-ol trifluoroacetate salt

This compound is prepared using method C, Example 3. ¹H NMR (400 MHz, DMSO-d6): δ 8.66 (b, 1H), 7.54 (s, 1H), 7.53 (d, 1H), 7.43 (d, 1H), 7.41 (s, 1H), 7.16 (s, 1H), 4.37 (t, 2H), 4.02 (s, 3H), 3.94 (s, 3H), 3.70 (m, 2H), 3.43 (m, 2H), 3.14 (m, 2H), 2.86 (s, 3H), MS (ES+): MZ 467 (M+1).

### Example 162

### 2-(6,7-Dimethoxy-4-methyl-quinazolin-2-ylamino)-6-{2-[(1-hydroxymethyl-3-methyl butyl)-amino]-ethoxy}-quinazolin-4-olTrifluoroacetate salt

This compound is prepared using method C, Example 3. ¹H NMR (400 MHz, DMSO-d6): δ 8.62 (b, 1H), 7.52 (s, 1H), 7.50 (d, 1H), 7.43 (d, 1H), 7.41 (s, 1H), 7.16 (s, 1H), 4.37 (t, 2H), 4.02 (s, 3H), 3.94 (s, 3H), 3.78 (m, 1H), 3.58 (m, 1H), 3.44 (m, 2H), 3.28 (m, 1H), 1.69 (m, 1H), 1.58 (m, 3H), 1.45 (m, 1H), 0.91 (m, 6H),MS (ES+): MZ 523 (M+1).

### Example 163

### 6-[2-(3-Amino-pyrrolidin-1-yl)-ethoxy]-2-(6,7-dimethoxy-4-methyl-quinazolin-2-ylamino)-quinazolin-4-ol

This compound is prepared using method C, Example 3. ¹H NMR (400 MHz, DMSO-d6): δ 8.23 (b, 3H), 7.57 (s, 1H), 7.52 (d, 1H), 7.44 (m, 2H), 7.16 (s, 1H), 4.43 (t, 2H), 4.02 (s, 3H), 3.94 (s, 3H), 3.71 (m, 7H), 2.86 (s, 3H), 1.90-2.60 (m, 2H), MS (ES+): MZ 492 (M+1).

### Example 164

### 2-(6,7-Dimethoxy-4-methyl-quinazolin-2-ylamino)-6-[2-(2-hydroxymethyl-pyrrolidin-1-yl)-ethoxy]-quinazolin-4-ol trifluoroacetate salt

This compound is prepared using method C, Example 3. ¹H NMR (400 MHz, DMSO-d6): δ 9.50 (b, 1H), 7.55 (s, 1H), 7.54 (d, 1H), 7.45 (d, 1H), 7.42 (s, 1H), 7.17 (s, 1H), 4.45 (t, 2H), 4.02 (s, 3H), 3.94 (s, 3H), 3.85 (m, 1H), 3.8 (m, 1H), 3.67 (m, 2H), 3.56 (m, 1H), 3.29 (m, 1H), 2.87 (s, 3H), 2.13 (m, 1H), 2.06 (m, 1H), 1.90 (m, 1H), 1.75 (m, 1H), MS (ES+): MZ 507 (M+1).

### Example 165

### 2-{2[-2-(6,7-Dimethoxy-4-methyl-quinazolin-2-ylamino)-4-hydroxy-quinazolin-6-yloxy]-ethylamino}-propane-1,3-diol trifluoroacetate salt

This compound is prepared using method C, Example 3. ¹H NMR (400 MHz, DMSO-d6): δ 8.62 (s, 1H), 7.53 (m, 2H), 7.44 (s, 1H), 7.43 (d, 1H), 7.17 (s, 1H), 4.46 (t, 1H), 4.02 (s, 3H), 3.96 (s, 3H), 2.86 (s, 3H), MS (ES+): MZ 497 (M+1).

### Example 166

### 2-(6,8-Dimethoxy-4-methyl-quinazolin-2-ylamino)-6-(2-pyrrolidin-1-yl-ethoxy)-quinazolin-4-ol trifluoroacetate salt

This compound is prepared using method C, Example 3. ¹H NMR (400 MHz, DMSO-d6): δ 10.32 (s, 1H), 7.55 (s, 1H), 7.45 (m, 2H), 7.08 (s, 1H), 7.03 (s, 1H), 4.44 (t, 2H), 4.03 (s, 3H), 3.92 (s, 3H), 3.63 (m, 4H), 3.13 (m, 2H), 2.86 (s, 3H), 1.87 (m, 4H). MS (ES+): MZ 477 (M+1).

### Example 167

### 2-(6,8-Dimethoxy-4-methyl-quinazolin-2-ylamino)-6-{2-[ethyl-(2-hydroxy-ethyl)-amino]-ethoxy}-quinazolin-4-ol trifluoroacetate salt

This compound is prepared using method C, Example 3. ¹H NMR (400 MHz, DMSO-d6): δ 9.42 (s, 1H), 7.55 (s, 1H), 7.43 (m, 2H), 7.08 (s, 1H), 7.03 (s, 1H), 4.46 (t, 1H), 4.02 (s, 3H), 3.93 (s, 3H), 3.79 (m, 2H), 3.61 (m, 2H), 3.32 (m, 4H), 2.86 (s, 3H), 1.28 (t, 3H), MS (ES+): MZ 495 (M+1).

### Example 168

### 2-(6,8-Dimethoxy-4-methyl-quinazolin-2-ylamino)-6-[2-(2-hydroxymethyl-pyrrolidin-1-yl)-ethoxy]-quinazolin-4-ol trifluoroacetate salt

This compound is prepared using method C, Example 3. ¹H NMR (400 MHz, DMSO-d6): δ 9.63 (b, 1H), 7.53 (s, 1H), 7.46 (s, 2H), 7.07 (s, 1H), 7.02 (s, 1H), 4.46 (t, 2H), 4.02 (s, 3H), 3.92 (s, 3H), 3.2-3.90 (m, 7H), 2.84 (s, 3H), 1.70-2.20 (m, 4H), MS (ES+): MZ 507 (M+1).

### Example 169 2-(6,7-Dimethoxy-4-methyl-quinazolin-2-ylamino)-6-[2-(2-hydroxy-1-methyl-ethylamino)-ethoxy]-quinazolin-4-ol trifluoroacetate salt

This compound is prepared using method C, Example 3. ¹H NMR (400 MHz, DMSO-d6): δ 7.46 (s, 1H), 7.40 (s, 2H), 7.36 (d, 1H), 7.09 (s, 1H), 4.16 (t, 1H), 4.00 (s, 3H), 3.93 (s, 3H), 2.80-3.60 (m, 5H), 2.84 (s, 3H), 1.10 (d, 3H), MS (ES+): MZ 481 (M+1).

### Example 170

### 2-(6,7-Dimethoxy-4-methyl-quinazolin-2-ylamino)-6-[2-(2-hydroxymethyl-morpholin-4-yl)-ethoxy]-quinazolin-4-ol trifluoroacetate salt

This compound is prepared using method C, Example 3. ¹H NMR (400 MHz, DMSO-d6): δ 10,76 (b, 1H), 9.05 (b, 1H), 7.53 (s, 1H), 7.50 (d, 1H), 7.42 (d, 1H), 7.39 (s, 1H), 7.14 (s, 1H), 4.49 (t, 1H), 3.95 (s, 3H), 3.92 (s, 3H), 3.0-3.90 (m, 11H), 2.84 (s, 3H), MS (ES): MZ 523 (M+1).

### Example 171

### 2-(6,7-Dimethoxy-4-methyl-quinazolin-2-ylamino)-6-{2[(2-dimethylamino-ethyl)-ethyl-amino]-ethoxy}-quinazolin-4-ol trifluoroacetate salt

This compound is prepared using method C, Example 3. 1H NMR (400 MHz, DMSO-d6): δ 7.54 (d, 1H), 7.48 (d, 1H), 7.42 (s, 1H), 7.42-7.38 (m, 1H), 7.14 (s, 1H), 4.38 (b m, 2H), 4.20-3.00 (b m, 8H), 4.00 (s, 3H), 3.94 (s, 3H), 2.84 (s, 9H), 1.22 (b m, 3H), MS (ES+): MZ 522 (M+1).

### Example 172

### 2-(6,7-Dimethoxy-4-methyl-quinazolin-2-ylamino)-6-[2-(monpholin-4-ylamino)-ethoxy-quinazolin-4-ol trifluoroacetate salt

This compound is prepared using method C, Example 3. ¹H NMR (400 MHz, DMSO-d6): δ 7.56-7.51 (m, 1H), 7.49 (d, 1H), 7.43-7.38 (m, 2H), 7.14 (s, 1H), 4.45 (b m, 2H), 4.00 (s, 3H), 3.97 (b s, 2H), 3.92 (s, 3H), 3.76-3.64 (m, 3H), 3.64-3.48 (m, 4H), 3.38-2.98 (m, 3H) 2.84 (s, 3H). MS (ES+): MZ 508 (M+1).

### Example 173

### 2-(6,7-Dimethoxy-4-methyl-quinazolin-2-ylamino)-6-[2-(2-hydroxymethyl-piperidin-1-yl)-ethoxy]-quinazolin-4-ol trifluoroacetate salt

This compound is prepared using method C, Example 3. ¹H NMR (400 MHz, DMSO-d6): δ 7.52 (d, 1H), 7.48 (d, 1H), 7.42-7.36 (m, 2H), 7.14 (s, 1H), 4.45 (m, 2H), 3.99 (s, 3H), 3.92 (s, 3H), 3.55-3.11 (m, 8H), 2.84 (s, 3H), 1.85-1.40 (m, 6H). MS (ES+): MZ 521 (M+1).

### Example 174

### 2-(6,7-Dimethoxy-4-methyl-quinazolin-2-ylamino)-6-(2-[1,2,4]triazol-4-yl-ethoxy)-quinazolin-4-ol trifluoroacetate salt

This compound is prepared using method C, Example 3. ¹H NMR (400 MHz, DMSO-d6): δ 8.62 (s, 1H), 8.00 (s, 1H), 7.47 (d, 1H), 7.38 (d, 1H), 7.36 (s, 1H), 7.31 (m, 1H), 7.16 (s, 1H), 4.62-4.58 (m, 2H), 4.45-4.40 (m, 2H), 3.98 (s, 3H), 3.90 (s, 3H), 2.85 (s, 3H). MS (ES+): MZ 475 (M+1).

### Example 175

### 2-(6,7-Dimethoxy-4-methyl-quinazolin-2-ylamino)-6-{2-[4-(2-hydroxy-ethyl)-piperazin-1-yl]-ethoxy}-quinazolin-4-ol trifluoroacetate salt

This compound is prepared using method C, Example 3. ¹H NMR (400 MHz, DMSO-d6): δ 7.52-7.45 (m, 2H), 7.42-7.35 (m 2H), 7.14 (s, 1H), 4.32 (b s, 2H), 3.99 (s, 3H), 3.92 (s, 3H), 3.71 (b m, 2H), 3.60-2.98 (m, 12H), 2.83 (s, 3H). MS (ES+): MZ 536 (M+1).

### Example 176

### 2-(6,7-Dimethoxy-4-methyl-quinazolin-2-ylamino)-6-{2-[N′-(2-hydroxy-ethyl)-hydrazino]-ethoxy}-quinazolin-4-ol trifluoroacetate salt

This compound is prepared using method C, Example 3. ¹H NMR (400 MHz, DMSO-d6): δ 7.49-7.43 (m, 2H), 7.40-7.34 (m, 2H), 7.13 (s, 1H), 4.28 (b m, 2H), 3.99 (s, 3H), 3.91 (s, 3H), 3.68 (b m, 2H), 3.58 (m, 1H), 3.38-3.31 (m, 2H), 3.13-3.01 (b s, 2H), 2.94 (m, 1H) 2.83 (s, 3H). MS (ES+): MZ 482 (M+1).

### Example 177

### 2-(6,7-Dimethoxy-4-methyl-quinazolin-2-ylamino)-6-[2-(2,6-dimethyl-morpholin-4-yl)-ethoxy]-quinazolin-4-ol trifluoroacetate salt

This compound is prepared using method C, Example 3. ¹H NMR (400 MHz, DMSO-d6): δ 7.52 (d, 1H), 7.47 (d, 1H), 7.41-7.35 (m, 2H), 7.12 (s, 1H), 4.44 (b s, 2H), 3.99 (s, 3H), 3.90 (s, 3H), 3.86 (m, 2H), 3.59-3.54 (m, 4H), 2.83 (s, 3H), 2.75 (m, 2H), 1.12 (d, 6H). MS (ES+): MZ 521 (M+1).

### Example 178

### 2-(6,7-Dimethoxy-4-methyl-quinazolin-2-ylamino)-6-{N′,N′-dimethyl-hydrazino]-ethoxy}-quinazolin-4-ol trifluoroacetate salt

This compound is prepared using method C, Example 3. ¹H NMR (400 MHz, DMSO-d6): δ 7.57-7.51 (m, 2H), 7.42 (d, 1H), 7.39 (s, 1H), 7.19 (s, 1H), 4.58 (b m, 2H), 3.99 (s, 3H), 3.93 (d, 2H), 3.91 (s, 3H), 3.34 (s, 6H), 2.85 (s, 3H).

MS (ES+): MZ 466 (M+1).

### Example 179

### 2-(6,7-Dimethoxy-4-methyl-quinazolin-2-ylamino)-6-{2-[2-(2-hydroxy-ethoxy)-ethylamino]-ethoxy}-quinazolin-4-ol trifluoroacetate salt

This compound is prepared using method C, Example 3. ¹H NMR (400 MHz, DMSO-d6): δ 8.75 (b s, 2H), 7.52-7.45 (m, 2H), 7.42-7.36 (m, 2H), 7.13 (s, 1H) 4.34 (m, 2H), 3.99 (s, 3H), 3.91 (s, 3H), 3.67 (m, 2H), 3.55-3.46 (m, 4H), 3.43 (m, 2H), 3.24 (m, 2H), 2.83 (s, 3H). MS (ES+): MZ 511 (M+1).

### Example 180

### 2-(6,7-Dimethoxy-4-methyl-quinazolin-2-ylamino)-6-[2-(2-pyrrolidin-1-ylmethyl-pyrrolidin-1-yl)-ethoxy]-quinazolin-4-ol trifluoroacetate salt

This compound is prepared using method C, Example 3. ¹H NMR (400 MHz, DMSO-d6): δ 7.53 (d, 1H), 7.48 (d, 1H), 7.44-7.37 (m, 2H), 7.13 (s, 1H), 4.42 (b m, 2H), 3.99 (s, 3H), 3.92 (s, 3H), 3.88-3.10 (m, 11 H), 2.83 (s, 3H), 2.36-1.78 (m, 8 H). MS (ES+): MZ 560 (M+1).

### Example 181

### 2-(6,7-Dimethoxy-4-methyl-quinazolin-2-ylamino)-6-[2-(2-hydroxy-cyclohexylamino)-ethoxy]-quinazolin-4-ol trifluoroacetate salt

This compound is prepared using method C, Example 3. ¹H NMR (400 MHz, DMSO-d6): δ 8.56 (b m, 1H), 8.42 (b m, 1H), 7.51 (d, 1H), 7.48 (d, 1H), 7.42-7.37 (m, 2H), 7.13 (s, 1H), 4.34 (m, 2H), 4.12 (b m, 1H), 4.00 (s, 3H), 3.92 (s, 3H), 3.46-3.32 (m, 2H), 3.24-3.14 (m, 1H), 2.83 (s, 3H), 1.83-1.14 (m, 8H). MS (ES+): MZ 521 (M+1).

### Example 182

### 2-(6,7-Dimethoxy-4-methyl-quinazolin-2-ylamino)-6-[2-(methyl-pyridin-3-ylmethyl-amino)-ethoxy]-quinazolin-4-ol trifluoroacetate salt

This compound is prepared using method C, Example 3. ¹H NMR (400 MHz, DMSO-d6): δ 8.77 (s, 1H), 8.69 (d, 1H), 8.06 (d, 1H), 7.57 (d, 1H), 7.53 (d, 1H), 7.50 (d, 1H), 7.43-7.37 (m, 2H), 7.15 (s, 1H), 4.60-4.42 (m, 4H), 4.00 (s, 3H), 3.92 (s, 3H), 3.57 (b s, 2H), 2.85 (s, 6H). MS (ES+): MZ 528 (M+1).

### Example 183

### 2-(6,7-Dimethoxy-4-methyl-quinazolin-2-ylamino)-6-[2-(2-pyridin-4-yl-ethylamino)-ethoxy]-quinazolin-4-ol TFA salt

This compound is prepared using method C, Example 3. NMR (400 MHz, DMSO-d6): δ 9.14 (b s, 2H), 8.84 (d, 2H), 7.85 (d, 2H), 7.58-7.54 (m, 2H), 7.46 (dd, 1H), 7.44 (s, 1H), 7.20 (s, 1H), 4.42 (m, 2H), 4.05 (s, 3H), 3.97 (s, 3H), 3.56-3.45 (m, 4H), 3.26 (m, 2H), 2.90 (s, 3H). MS (ES+): MZ 528 (M+1).

### Example 184

### 2-(6,7-Dimethoxy-4-methyl-quinazolin-2-ylamino)-6-[2-(methyl-pyridin-4-ylmethyl-amino)-ethoxy]-quinazolin-4-ol TFA salt

This compound is prepared using method C, Example 3. NMR (400 MHz, DMSO-d6): δ 8.78 (d, 2H), 7.70 (d, 2H), 7.57-7.51 (m, 2H), 7.47-7.42 (m, 2H), 7.19 (s, 1H), 4.56-4.46 (m, 4H), 4.04 (s, 3H), 3.96 (s, 3H), 3.61-3.53 (m, 2H), 2.89 (s, 3H), 2.88 (s, 3H). MS (ES+): MZ 528 (M+1).

### Example 185

### High-Throughput Screening

A platform of fluorescence-based DNA replication assays using reconstituted replication systems from a variety of bacterial pathogens has been developed to an automated high-throughput screening (HTS) format. The resultant HTS assays were used to screen small molecule compound libraries to identify inhibitors of bacterial DNA replication in vitro.

The assays use the dsDNA-specific dye PicoGreen to monitor DNA synthesis. The HTS campaigns are conducted using a Beckman-Coulter Biomek® FX liquid handling workstation equipped with a 384-well pipeting head and plate stackers, allowing automated assays in 384-well plates. Generally, test compounds (0.5 to 2 μL, 400 μM) and enzyme mixes are combined and incubated at 23° C. for 5 minutes prior to the addition of nucleotide substrates and DNA templates (˜5 μL) in a final assay volume of 20-30 μl. Positive controls (DMSO only, with no test compound) and negative controls (containing EDTA, which blocks the reaction by chelating essential Mg2+) are included in the outer 2 columns each assay plate. The remaining 320 wells were used for assay of test compounds. Reactions are allowed to proceed to an endpoint (determined for each HTS assay; normally 10 to 20 minutes) by addition of solutions containing EDTA and PicoGreen dye reagent. Detection of the PicoGreen-stained, dsDNA reaction product is performed using a Perkin-Elmer Victor²V™ Fluorescence plate reader using an excitation wavelength of 480 nm and emission wavelength of 520 nm. HTS data is analyzed using ActivityBase software (IDBS Ltd., Guildford, UK) to convert the raw fluorescence units into normalized % activity. Compounds displaying activity ≦50% of the mean DMSO control % activity are scored as hits. Additionally, ActivityBase is used to calculate and track overall data quality (Z- and Z′-factors) and signal to background ratios. Z- and Z-factors are consistently above 0.7.

Hit compounds identified in HTS are retested in triplicate in a modified HTS assay using the Amersham-Pharmacia Biotech Scintillation Proximity assay (SPA) detection technology in order to eliminate false positive hits potentially interfering with the PicoGreen assay readout. This assay measures incorporation of [³H]-dTTP into replicated DNA. A primary hit compound is considered to be confirmed only if it scored as a hit in 2 out of 3 retests in the SPA format assay. The potency and specificity of hit compounds are further characterized using a number of different assays.

Enzymes, proteins and DNA templates required for each HTS campaign are purified in amounts sufficient to yield material generally adequate for >10⁶ data points. Optimal HTS assay conditions have been determined by varying pH and titrating buffer components including salt, divalent cation (Mg²⁺), reducing agents, detergents, and glycerol. All protein components are titrated individually in the presence of an excess of the others in order to determine linear activity ranges for each component in the HTS format. The concentration of each protein is set to where it will become limiting if bound by an inhibitor, making the assay sensitive to interference with any of the system's components. All nucleotide substrates (dNTP's and rNTP's) are also titrated to determine the concentrations that permitted optimal DNA replication without providing an excess that could mask the action of competitive inhibitors of nucleoside triphosphate incorporation during primer RNA or DNA synthesis or ATP hydrolysis.

Since HTS requires the preparation of large batches of enzyme mix that will be used over several hours, enzyme stability is determined by monitoring enzyme mix inactivation kinetics at 23° C. and 4° C. Despite its enormous complexity, the DNA replication systems that have been reconstituted are generally robust, producing signals that are sufficiently stable over the course of at least one full screening day when stored at 4° C. To enable detection of inhibitors with rapid or slow binding kinetics, the HTS assays were designed enable preincubation of target enzymes with compounds prior to the addition of nucleotide substrates. In addition to simple enzyme stability and assay response in the presence of DMSO, enzyme-inactivation kinetics in the presence of DMSO have also been determined for each HTS assay that has been developed. In general, the HTS systems can tolerate between 8-20% DMSO for at least 2 minutes up to a maximum of 2 hours without significantly compromising the reaction.

The bis-quinazoline compounds of the present have potent, reversible biochemical activity against DNA replication complexes from both Gram-positive and Gram-negative bacteria, with IC₅₀ values ranging from <50 nM to 1 μM across the series. The enzyme target within the DNA replication complex and kinetic mechanism of inhibition has been determined for the series (Ki values <1 μM, for selected compounds). These compounds show selectivity for bacterial replication targets (as compared to the eukaryotic equivalents).

Representative bis-quniazolines were tested in reconstituted bacterial DNA polymerase holoenzymes from E. coli, Y. pestis and S. pyogenes which exhibited reversible biochemical activity against DNA replication complexes from both Gram-positive and Gram-negative bacteria, with IC₅₀ values ranging from <50 nM to 1 μM across the series.

### Example 186

### Demonstration of Antibacterial Effect

Representative bis-quinazolines were tested for antibacterial activity using the both microdilution method to determine their minimum inhibitory concentrations (MICs). All compounds were tested using standard methods in accordance with NCCLS guidelines (National Committee for Clinical Laboratory Standards. 2003. Methods for dilution antimicrobial susceptibility testing for bacteria that grow aerobically. Approved Standard M-7 A-6, NCCLS, Wayne, Pa.). The bacterial strains used in the susceptibility tests were obtained from the American Type Culture Collection (ATCC) and included resistant phenotypes such as oxacillin-resistant S. aureus (ORSA) and vancomycin-resistant enterococci (VRE). The minimum bactericidal concentrations (MBCs) were determined for selected organisms. The effect of protein binding on the antibacterial activity of representative bis-quinazolines were determined by conducting MIC tests in the presence of human serum. The bis-quinazoline compounds demonstrated antibacterial and bactericidal activity against clinically important bacterial pathogens in the presence of 50% human serum.

### Example 187

### Demonstration of the Antibacterial Activity of 2-(6-Methoxy-4-methyl-quinazolin-2-ylamino)-3H-quinazolin-4-ol when Tested in an S. aureus Infection in Mice

Single Dose Toxicity

A single dose toxicity study was conducted in the mouse prior to evaluating the compound for in vivo efficacy. 2-(6-Methoxy-4-methyl-quinazolin-2-ylamino)-3H-quinazolin-4-ol was prepared in a vehicle consisting of 10% DMSO, 10% ethanol and 80% saline that contained 3% β-cyclodextrin. The compound was administered as a single dose to the peritoneal cavity of female Swiss-Webster mice at the six concentrations (2.5, 5, 10, 15, 20 and 25 mg/kg) proposed for evaluation in the efficacy study. The mice were monitored for toxicity for one week following compound administration. All mice tolerated the compound and vehicle at all the concentrations tested and showed no signs of discomfort one week after dosing.

Single Dose Efficacy

The S. aureus murine intra-abdominal sepsis model was used to evaluate the in vivo efficacy of 2-(6-Methoxy-4-methyl-quinazolin-2-ylamino)-3H-quinazolin-4-ol. Female Swiss-Webster mice (weighing 25 grams, 10 per treatment group) were infected with the minimum lethal dose (MLD) of S. aureus ATCC 29213 (10⁹ CFU) by inoculating the intraperitoneal cavity of the animal. The mice then received the compound (2.5, 5, 10, 15, 20 and 25 mg/kg) by intraperitonal injection one hour post infection. The animals were then monitored at frequent intervals and the numbers of mice surviving in the different treatment groups was scored and recorded in an attempt to determine the protective dose (PD₅₀). Levofloxacin was evaluated as a positive control (0.875, 1.75, 3.125, 6.25, 12.5 and 25 mg/kg). Vehicle only and saline controls were also evaluated.

2-(6-Methoxy-4-methyl-quinazolin-2-ylamino)-3H-quinazolin-4-ol at 25 mg/kg was effective in protecting 50% of the mice following infection with the MLD of S. aureus.

1. A compound of formula (I): 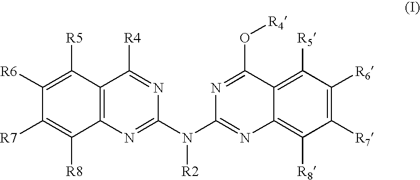
wherein
R₂, R₄, and R₄′ are independently selected from the group consisting of hydrogen, alkyl, alkenyl, alkynyl, CH₂-aryl, CH₂-heterocyclic, and phenyl; and
wherein
R₅, R₅′, R₆, R₆′, R₇, R₇′, R₈, and R₈′ are independently selected from the group consisting of hydrogen, halo, nitro, cyano, and hydroxy, C₍₁₋₇₎alkyl (optionally substituted by halo, hydroxy, amino), mono to perfluoro C₍₁₋₃₎alkyl, C₍₃₋₇₎cycloalkyl, C₍₁₋₇₎alkoxy, amino, mono- or di-C₍₁₋₆₎alkylamino, C₍₁₋₇₎alkylthio, benzyloxy, alkenyl, O-alkenyl, S-alkenyl, NH-alkenyl, alkynyl, O-alkynyl, S-alkynyl, NH-alkynyl, CH₂-aryl, CH₂-heterocyclic, aryl, heterocyclic, S-aryl, O-aryl, NH-aryl, phenyl, O-carbohydrate, S-carbohydrate, NH-carbohydrate, sulfamoyl, carbamoyl, N-alkyl fused to aryl or heterocyclic, OCOR₉, SCOR₉, NHCOR₉, NHR₉R₁₀; wherein R₉ and R₁₀ are independently selected from the group consisting of alkyl, aryl, including heterocyclic, and acyl donors selected from the group consisting of alkyl acids, amino acids, and aromatic acids;
and a cyclic ring connecting any of R₅ and R₆; R₆ and R₇; R₇ and R₈; R₅′ and R₆′; R₆′ and R₇′; and R₇′ and R₈′, wherein the cyclic ring is selected from the group consisting of methylenedioxy, dioxane, substituted methylenedioxy, heteroatom five member ring, and heteroatom six member ring; and
wherein
R₆, R₇, and R₆′ may also be further independently selected from the group consisting of Linker-Q,
wherein
Linker is a C₁₋₁₀ straight chain or branched alkyl, alkenyl, or alkynyl; wherein one or more methylene groups are optionally independently replaced by O, N or S; and wherein said linker is optionally substituted with 0-2 oxo groups and one or more C₁₋₄ branched or unbranched alkyl which may be substituted by one or more halogen atoms; and
wherein
Q is selected from the group consisting of phenyl; naphthyl; pyridine; pyrimidine; pyridazine; imidazole; benzimidazole; pyrazole; triazole; furan; thiophene; pyran; naphthyridine; oxazo[4,5-b]pyridine; and imidazo[4,5-b]pyridine; each of which may be optionally substituted with one to three groups selected from the group of halogen, C₁₋₆ alkyl, C₁₋₆ alkoxy, hydroxy, mono- or di-(C₁₋₃ alkyl)amino, C₁₋₆ alkyl-S(O)ₘ and phenylamino, wherein the phenyl ring is optionally substituted with one to two groups from the group halogen, C₁₋₆ alkyl and C₁₋₆ alkoxy;
tetrahydropyran; tetrahydrofuran; 1,3-dioxolanone; 1,3-dioxanone; 1,4-dioxane; morpholine; thiomorpholine; thiomorpholine sulfoxide; thiomorpholine sulfone; piperidine; piperazine; piperidinone; pyrrolidine; tetrahydrpyrimidone; cyclohexanone; cyclohexanol; each of which may be optionally substituted with one to three groups selected from the group consisting of C₁₋₆ alkyl, C₁₋₆ alkoxy, hydroxy, mono- or di-(C₁₋₃ alkyl)amino-C₁₋₃ alkyl, phenylamino-C₁₋₃ alkyl and C₁₋₃ alkoxy-C₁₋₃ alkyl;
C₁₋₆ alkoxy; secondary or tertiary amine wherein the amino nitrogen is covalently bonded to groups selected from the group consisting of C₁₋₆ alkyl, C₁₋₅ alkoxyalkyl, C₁₋₆ branched alkyl, each of which are optionally substituted with halo, hydroxy, or amino;
secondary or tertiary amine wherein the amino nitrogen is covalently bonded to phenyl, C₁₋₂ alkylphenyl or C₁₋₂ alkylpyridyl, wherein the aryl ring is optionally substituted with one to two groups selected from the group consisting of halogen, C₁₋₆ alkoxy, and hydroxy.

2. A compound of claim 1, wherein linker is selected from the group consisting of ethoxy, methoxy, or propoxy.

3. A compound of claim 1, wherein said compound is selected from the group consisting of 6-Methoxy-4-methyl-quinazolin-2-yl)-(4-methoxy-quinazolin-2-yl)-methyl-amine;
2-[(6-Methoxy-4-methyl-quinazolin-2-yl)-methyl-amino]-quinazolin-4-ol;
2-(4-Fluoromethyl-6-methoxy-quinazolin-2-ylamino)-quinazolin-4-ol;
2-(4-Ethyl-6-methoxy-quinazolin-2-ylamino)-quinazolin-4-ol;
2-(6-Methoxy-4-propyl-quinazolin-2-ylamino)-quinazolin-4-ol;
2-(4-Fluoromethyl-6,7-dimethoxy-quinazolin-2-ylamino)-8-methoxy-quinazolin-4-ol; and
2-(4-Fluoromethyl-6,7-dimethoxy-quinazolin-2-ylamino)-5-methoxy-quinazolin-4-ol.

4. The compound of claim 1, wherein said compound is a compound of formula (II): 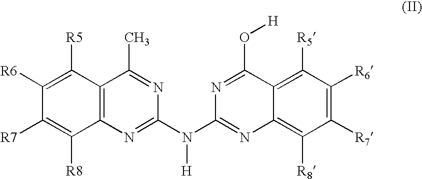
wherein
R₅, R₅′, R₆, R₆′, R₇, R₇′, R₈, and R₈′ are independently selected from the group consisting of hydrogen; halo; hydroxy; C₍₁₋₇₎alkyl (optionally substituted by halo, hydroxy, amino); mono to perfluoro C₍₁₋₃₎alkyl; C₍₃₋₇₎cycloalkyl; C₍₁₋₇₎alkoxy; amino; and mono- or di-C₍₁₋₆₎alkylamino; and a cyclic ring connecting any of R₅ and R₆; R₆ and R₇; R₇ and R₈; R₅′ and R₆′; R₆′ and R₇′; and R₇′ and R₈′, wherein the cyclic ring is selected from the group consisting of methylenedioxy, dioxane, substituted methylenedioxy, heteroatom five member ring, and heteroatom six member ring; and
wherein
R₆, R₇, and R₆′ may also be further independently selected from the group consisting of Linker-Q,
wherein
Linker is a C₁₋₆ straight chain or branched alkyl, alkenyl, or alkynyl, wherein one or more methylene groups are optionally independently replaced by O, N or S; and wherein said Linker is optionally substituted with 0-2 oxo groups and one or more C₁₋₄ branched or unbranched alkyl which may be substituted by one or more halogen atoms; and
Q is selected from the group consisting of pyridine, pyrazole, triazole, morpholine, piperidine, piperazine, pyrrolidine, each of which may be optionally substituted;
C₁₋₄ alkoxy; secondary or tertiary amine wherein the amino nitrogen is covalently bonded to groups selected from the group consisting of C₁₋₄ alkyl, C₁₋₅ alkoxyalkyl, and C₁₋₆ branched alkyl, each of which may be optionally substituted with halo, hydroxy, or amino;
secondary or tertiary amine wherein the amino nitrogen is covalently bonded to groups selected from phenyl, C₁₋₂ alkylphenyl and C₁₋₂ alkylpyridyl, wherein the aryl ring is optionally substituted with one to two groups selected from the group consisting of halogen, C₁₋₃ alkoxy, and hydroxy:

5. A compound of claim 4, wherein said compound is selected from the group consisting of 2-(4-Hydroxy-quinazolin-2-ylamino)-4-methyl-quinazolin-6-ol;
6,7,8-Trimethoxy-2-(6-methoxy-4-methyl-quinazolin-2-ylamino)-quinazolin-4-ol trifluoroacetate salt;
2-(6-Methoxy-4-methyl-quinazolin-2-ylamino)-6,8-dimethyl-quinazolin-4-ol;
2-(6-Methoxy-quinazolin-2-ylamino)-8-methyl-quinazolin-4-ol;
2-(6-Methoxy-4-methyl-quinazolin-2-ylamino)-8-methyl-6-nitro-quinazolin-4-ol;
6-Amino-2-(6-methoxy-4-methyl-quinazolin-2-ylamino)-8-methyl-quinazolin-4-ol trifluoroacetate salt;
6-N-methylamino-2-(6-methoxy-4-methyl-quinazolin-2-ylamino)-8-methyl-quinazolin-4-ol trifluoroacetate salt;
6-N-dimethylamino-2-(6-methoxy-4-methyl-quinazolin-2-ylamino)-8-methyl-quinazolin-4-ol trifluoroacetate salt;
7-Fluoro-2-(6-methoxy-4-methyl-quinazolin-2-ylamino)-6-nitro-quinazolin-4-ol trifluoroacetate salt;
2-(8-Methoxy-4-methyl-5-nitro-quinazolin-2-ylamino)-quinazolin-4-ol trifluoroacetate salt;
2-(5,8-Dimethoxy-4-methyl-quinazolin-2-ylamino)-quinazolin-4-ol trifluoroacetate salt;
2-(5,6,7-Trimethoxy-4-methyl-quinazolin-2-ylamino)-quinazolin-4-ol trifluoroacetate salt;
2-(5-Methoxy-4,8-dimethyl-quinazolin-2-ylamino)-quinazolin-4-ol trifluoroacetate salt;
2-(8-Methoxy-4-methyl-5-nitro-quinazolin-2-ylamino)-6,8-dimethyl-quinazolin-4-ol, trifluoroacetate salt;
4-Hydroxy-2-(6-methoxy-4-methyl-quinazolin-2-ylamino)-quinazoline-7-carboxylic acid;
2-(7-Fluoro-6-methoxy-4-methyl-quinazolin-2-ylamino)-quinazolin-4-ol trifluoroacetate salt;
6-Chloro-2-(6-methoxy-4-methyl-quinazolin-2-ylamino)-quinazolin-4-ol;
2-(4-Methyl-6-methylsulfanyl-quinazolin-2-ylamino)-quinazolin-4-ol trifluoroacetate salt;
6,8-Dimethyl-2-(5,6,7-trimethoxy-4-methyl-quinazolin-2-ylamino)-quinazolin-4-ol trifluoroacetate salt;
5-Methoxy-2-(6-methoxy-4-methyl-quinazolin-2-ylamino)-quinazolin-4-ol trifluoroacetate salt;
N-[4-Hydroxy-2-(6-methoxy-4-methyl-quinazolin-2-ylamino)-quinazolin-6-yl]-acetamide trifluoroacetate salt;
8-Bromo-2-(6-methoxy-4-methyl-quinazolin-2-ylamino)-6-methyl-quinazolin-4-ol trifluoroacetate salt;
2-(5-Methoxy-4,8-dimethyl-quinazolin-2-ylamino)-6,8-dimethyl-quinazolin-4-ol trifluoroacetate salt;
2-(6,7-Dimethoxy-4-methyl-quinazolin-2-ylamino)-6,8-dimethyl-quinazolin-4-ol trifluoroacetate salt;
2-(2,2-Difluoro-8-methyl-[1,3]dioxolo[4,5-g]quinazolin-6-ylamino)-quinazolin-4-ol trifluoroacetate salt;
2-(5,8-Dimethoxy-4-methyl-quinazolin-2-ylamino)-6,8-dimethyl-quinazolin-4-ol trifluoroacetate salt;
2-(7-Fluoro-6-methoxy-4-methyl-quinazolin-2-ylamino)-6,8-dimethyl-quinazolin-4-ol trifluoroacetate salt;
2-(6,8-Dimethoxy-4-methyl-quinazolin-2-ylamino)-8-methyl-quinazolin-4-ol trifluoroacetate salt;
6-Amino-2-(6-methoxy-4-methyl-quinazolin-2-ylamino)-quinazolin-4-ol; trifluoroacetate salt;
2-(8-Methoxy-4-methyl-quinazolin-2-ylamino)-6,8-dimethyl-quinazolin-4-ol;
2-(8-Methoxy-4-methyl-quinazolin-2-ylamino)-8-methyl-quinazolin-4-ol;
2-(6,7-Dimethoxy-4-methyl-quinazolin-2-ylamino)-quinazolin-4-ol;
2-(6-Methoxy-4-methyl-quinazolin-2-ylamino)-6-nitro-quinazolin-4-ol;
2-(6-Methoxy-4-methyl-quinazolin-2-ylamino)-7-nitro-quinazolin-4-ol;
5-Fluoro-2-(6-methoxy-4-methyl-quinazolin-2-ylamino)-quinazolin-4-ol;
6-Fluoro-2-(6-methoxy-4-methyl-quinazolin-2-ylamino)-quinazolin-4-ol;
7-Fluoro-2-(6-methoxy-4-methyl-quinazolin-2-ylamino)-quinazolin-4-ol;
2-(8-Methoxy-4-methyl-quinazolin-2-ylamino)-quinazolin-4-ol;
2-(8-Ethoxy-4-methyl-quinazolin-2-ylamino)-quinazolin-4-ol;
2-(6-Methoxy-4-methyl-quinazolin-2-ylamino)-quinazoline-4,6-diol;
6-Methoxy-2-(6-methoxy-4-methyl-quinazolin-2-ylamino)-quinazolin-4-ol;
6,7-Dimethoxy-2-(6-methoxy-4-methyl-quinazolin-2-ylamino)-quinazolin-4-ol;
2-(6-Methoxy-4-methyl-quinazolin-2-ylamino)-6-methyl-quinazolin-4-ol;
2-(6-Methoxy-4-methyl-quinazolin-2-ylamino)-7-methyl-quinazolin-4-ol;
2-(6-Methoxy-4-methyl-quinazolin-2-ylamino)-8-methyl-quinazolin-4-ol;
2-(6,7-Dimethoxy-4-methyl-quinazolin-2-ylamino)-8-methyl-6-nitro-quinazolin-4-ol trifluoroacetate salt;
2-(6,8-Dimethoxy-4-methyl-quinazolin-2-ylamino)-8-methyl-6-nitro-quinazolin-4-ol trifluoroacetate salt;
2-(8-Methoxy-4-methyl-quinazolin-2-ylamino)-8-methyl-6-nitro-quinazolin-4-ol trifluoroacetate salt;
2-[6-(2-Methoxy-ethoxy)-4-methyl-quinazolin-2-ylamino]-8-methyl-6-nitro-quinazolin-4-ol trifluoroacetate salt;
6-Amino-2-[6-(2-methoxy-ethoxy)-4-methyl-quinazolin-2-ylamino]-8-methyl-quinazolin-4-ol trifluoroacetate salt;
2-(6-Methoxy-4-methyl-quinazolin-2-ylamino)-quinazoline-4,8-diol trifluoroacetate salt;
2-(8-Methoxy-4-methyl-quinazolin-2-ylamino)-quinazoline-4,8-diol trifluoroacetate salt;
2-(7,8-Dimethoxy-4-methyl-quinazolin-2-ylamino)-6,8-dimethyl-quinazolin-4-ol trifluoroacetate salt;
2-(7,8-Dimethoxy-4-methyl-quinazolin-2-ylamino)-quinazolin-4-ol trifluoroacetate salt;
2-(6,8-Dimethoxy-4-methyl-quinazolin-2-ylamino)-6,8-dimethyl-quinazolin-4-ol trifluoroacetate salt;
2-(6-methoxy-7-amino-4-methyl-quinazolin-2-ylamino)-8-methoxy-quinazolin-4-ol trifluoroacetate salt;
2-(6-methoxy-7-nitro-4-methyl-quinazolin-2-ylamino)-8-methoxy-quinazolin-4-ol trifluoroacetate salt;
2-(7,8-Dimethoxy-4-methyl-quinazolin-2-ylamino)-8-methoxy-quinazolin-4-ol trifluoroacetate salt;
8-Methoxy-2-[6-methoxy-8-(2-methoxy-ethoxy)-4-methyl-quinazolin-2-ylamino]-quinazolin-4-ol trifluoroacetate salt;
8-Methoxy-2-[6-(2-methoxy-ethoxy)-4-methyl-quinazolin-2-ylamino]-quinazolin-4-ol trifluoroacetate salt;
8-Methoxy-2-[7-methoxy-8-(2-methoxy-ethoxy)-4-methyl-quinazolin-2-ylamino]-quinazolin-4-ol trifluoroacetate salt;
8-Methoxy-2-(8-methyl-2,3-dihydro-1,4-dioxa-5,7-diaza-phenanthren-6-ylamino)-quinazolin-4-ol;
5-ethoxy-2-(8-methyl-2,3-dihydro-1,4-dioxa-5,7-diaza-phenanthren-6-ylamino)-quinazolin-4-ol trifluoroacetate salt;
2-(6,7-Dimethoxy-4-methyl-quinazolin-2-ylamino)-5-methoxy-quinazolin-4-ol trifluoroacetate salt;
2-(6,8-Dimethoxy-4-methyl-quinazolin-2-ylamino)-5-methoxy-quinazolin-4-ol trifluoroacetate salt;
2-(7-Fluoro-6-methoxy-4-methyl-quinazolin-2-ylamino)-8-methoxy-quinazolin-4-ol trifluoroacetate salt;
5-Methoxy-2-[6-(2-methoxy-ethoxy)-4-methyl-quinazolin-2-ylamino]-quinazolin-4-ol trifluoroacetate salt;
2-[6,7-Bis-(2-methoxy-ethoxy)-4-methyl-quinazolin-2-ylamino]-8-methoxy-quinazolin-4-ol trifluoroacetate salt;
8-Methoxy-2-(5-methoxy-4,8-dimethyl-quinazolin-2-ylamino)-quinazolin-4-ol trifluoroacetate salt;
8-Methyl-2-(5-methoxy-4,8-dimethyl-quinazolin-2-ylamino)-quinazolin-4-ol trifluoroacetate salt;
2-(6,8-Dimethoxy-4-methyl-quinazolin-2-ylamino)-quinazoline-4,5-diol trifluoroacetate salt;
2-(7-Fluoro-6-methoxy-4-methyl-quinazolin-2-ylamino)-5-methoxy-quinazolin-4-ol trifluoroacetate salt;
5,8-Dimethoxy-2-(8-methyl-2,3-dihydro-1,4-dioxa-5,7-diaza-phenanthren-6-ylamino)-quinazolin-4-ol trifluoroacetate salt;
2-(6,7-Dimethoxy-4-methyl-quinazolin-2-ylamino)-8-fluoro-quinazolin-4-ol trifluoroacetate salt;
2-(6,7-Dimethoxy-4-methyl-quinazolin-2-ylamino)-8-ethoxy-quinazolin-4-ol trifluoroacetate salt;
2-(6,8-Dimethoxy-4-methyl-quinazolin-2-ylamino)-8-fluoro-quinazolin-4-ol trifluoroacetate salt;
2-(6,7-Dimethoxy-4-methyl-quinazolin-2-ylamino)-5,8-dimethoxy-quinazolin-4-ol trifluoroacetate salt;
2-(6,8-Dimethoxy-4-methyl-quinazolin-2-ylamino)-5,8-dimethoxy-quinazolin-4-ol trifluoroacetate salt;
[2-(6,7-Dimethoxy-4-methyl-quinazolin-2-ylamino)-4-hydroxy-quinazolin-6-yloxy]-acetonitrile;
2-(6,7-Dimethoxy-4-methyl-quinazolin-2-ylamino)-6-(2-methoxy-ethoxy)-quinazolin-4-ol trifluoroacetate salt;
2-(4-Hydroxy-8-methoxy-quinazolin-2-ylamino)-6-methoxy-4-methyl-quinazolin-7-ol trifluoroacetate salt;
2-(8-Fluoro-4-hydroxy-quinazolin-2-ylamino)-6-methoxy-4-methyl-quinazolin-7-ol trifluoroacetate salt;
6-(2-Chloro-ethoxy)-2-(6,7-dimethoxy-4-methyl-quinazolin-2-ylamino)-quinazolin-4-ol trifluoroacetate salt;
6-(3-Chloro-propoxy)-2-(6,7-dimethoxy-4-methyl-quinazolin-2-ylamino)-quinazolin-4-ol trifluoroacetate salt;
6-(2-Chloro-ethoxy)-2-(6,8-dimethoxy-4-methyl-quinazolin-2-ylamino)-quinazolin-4-ol trifluoroacetate salt;
8-Fluoro-2-(4-methyl-7,8-dihydro-[1,4]dioxino[2,3-g]quinazolin-2-ylamino)-quinazolin-4-ol trifluoroacetate salt;
7-(6,7-Dimethoxy-4-methyl-quinazolin-2-ylamino)-1,3-dioxa-6,8-diaza-cyclopenta[a]naphthalen-9-ol trifluoroacetate salt;
2-(6,7-Dimethoxy-4-methyl-quinazolin-2-ylamino)-5-isopropoxy-quinazolin-4-ol trifluoroacetate salt;
2-(6,8-Dimethoxy-4-methyl-quinazolin-2-ylamino)-5-isopropoxy-quinazolin-4-ol trifluoroacetate salt;
2-(6,8-Dimethoxy-4-methyl-quinazolin-2-ylamino)-5-ethoxy-quinazolin-4-ol trifluoroacetate salt;
2-(6,7-Dimethoxy-4-methyl-quinazolin-2-ylamino)-6-piperidin-1-yl-quinazolin-4-ol trifluoroacetate salt;
2-(6,7-Dimethoxy-4-methyl-quinazolin-2-ylamino)-6-morpholin-4-yl-quinazolin-4-ol trifluoroacetate salt;
2-[6-(2-Diethylamino-ethoxy)-4-methyl-quinazolin-2-ylamino]-8-methoxy-quinazolin-4-ol trifluoroacetate salt;
8-Methoxy-2-(6,7,8-trimethoxy-4-methyl-quinazolin-2-ylamino)-quinazolin-4-ol trifluoroacetate salt;
2-(7-Fluoro-8-methoxy-4-methyl-quinazolin-2-ylamino)-8-methoxy-quinazolin-4-ol trifluoroacetate salt;
8-Fluoro-2-(6,7,8-trimethoxy-4-methyl-quinazolin-2-ylamino)-quinazolin-4-ol trifluoroacetate salt;
8-Fluoro-2-(7-fluoro-8-methoxy-4-methyl-quinazolin-2-ylamino)-quinazolin-4-ol trifluoroacetate salt;
6-Amino-2-(6,7-dimethoxy-4-methyl-quinazolin-2-ylamino)-8-methyl-quinazolin-4-ol trifluoroacetate salt;
2-(6,7-Dimethoxy-4-methyl-quinazolin-2-ylamino)-6-dimethylamino-8-methyl-quinazolin-4-ol trifluoroacetate salt;
8-Amino-2-(6-methoxy-4-methyl-quinazolin-2-ylamino)-quinazolin-4-ol trifluoroacetate salt;
6-Amino-2-(6-methoxy-4-methyl-quinazolin-2-ylamino)-8-methoxy-quinazolin-4-ol trifluoroacetate salt;
6-Amino-2-(6,8-dimethoxy-4-methyl-quinazolin-2-ylamino)-8-methoxy-quinazolin-4-ol trifluoroacetate salt;
6-Amino-2-[6-(2-diethylamino-ethoxy)-4-methyl-quinazolin-2-ylamino]-8-methyl-quinazolin-4-ol trifluoroacetate salt;
6-Amino-2-(6,8-dimethoxy-4-methyl-quinazolin-2-ylamino)-8-methyl-quinazolin-4-ol trifluoroacetate salt;
2-(7-Dimethylamino-6-methoxy-4-methyl-quinazolin-2-ylamino)-8-methoxy-quinazolin-4-ol trifluoroacetate salt;
2-(6,7-Dimethoxy-4-methyl-quinazolin-2-ylamino)-5-dimethylamino-quinazolin-4-ol trifluoroacetate salt;
2-(6,7-Dimethoxy-4-methyl-quinazolin-2-ylamino)-6-(2H-tetrazol-5-ylmethoxy)-quinazolin-4-ol trifluoroacetate salt;
7-(6,8-Dimethoxy-4-methyl-quinazolin-2-ylamino)-1,3-dioxa-6,8-diaza-cyclopenta[a]naphthalen-9-ol trifluoroacetate salt;
7-(6,8-Dimethoxy-4-methyl-quinazolin-2-ylamino)-1,3-dioxa-6,8-diaza-cyclopenta[a]naphthalen-9-ol HCl salt;
2-(6,7-Dimethoxy-4-methyl-quinazolin-2-ylamino)-5,6-dimethoxy-quinazolin-4-ol TFA salt;
2-(6,7-Dimethoxy-4-methyl-quinazolin-2-ylamino)-5,6-dimethoxy-quinazolin-4-ol HCl salt;
2-(6,8-Dimethoxy-4-methyl-quinazolin-2-ylamino)-5,6-dimethoxy-quinazolin-4-ol TFA salt;
2-(6,8-Dimethoxy-4-methyl-quinazolin-2-ylamino)-5,6-dimethoxy-quinazolin-4-ol HCl salt;
2-(6,7-Dimethoxy-4-methyl-quinazolin-2-ylamino)-6-(2-hydroxy-ethoxy)-quinazolin-4-ol trifluoroacetate salt;
2-(6,8-Dimethoxy-4-methyl-quinazolin-2-ylamino)-6-(2-hydroxy-ethoxy)-quinazolin-4-ol trifluoroacetate salt;
6-[2-(3-Hydroxy-pyrrolidin-1-yl)-ethoxy]-2-(8-methoxy-4-methyl-quinazolin-2-ylamino)-quinazolin-4-ol trifluoroacetate salt;
2-(6,7-Dimethoxy-4-methyl-quinazolin-2-ylamino)-6-{2-[(2-hydroxy-ethyl)-methyl-amino]-ethoxy}-quinazolin-4-ol trifluoroacetate salt;
2-(6,8-Dimethoxy-4-methyl-quinazolin-2-ylamino)-6-{2-[(2-hydroxy-ethyl)-methyl-amino]-ethoxy}-quinazolin-4-ol trifluoroacetate salt;
2-(6,8-Dimethoxy-4-methyl-quinazolin-2-ylamino)-6-(2-morpholin-4-yl-ethoxy)-quinazolin-4-ol trifluoroacetate salt;
2-(6,7-Dimethoxy-4-methyl-quinazolin-2-ylamino)-6-[2-(1-hydroxymethyl-2-methyl-propylamino)-ethoxy]-quinazolin-4-ol Trifluoroacetate salt;
2-(6,8-Dimethoxy-4-methyl-quinazolin-2-ylamino)-6-[2-(2-hydroxy-1-methyl-ethylamino)-ethoxy]-quinazolin-4-ol trifluoroacetate salt;
6-(2-Diethylamino-ethoxy)-2-(6,8-dimethoxy-4-methyl-quinazolin-2-ylamino)-quinazolin-4-ol trifluoroacetate salt;
2-(6,7-Dimethoxy-4-methyl-quinazolin-2-ylamino)-6-(2-piperidin-1-yl-ethoxy)-quinazolin-4-ol trifluoroacetate salt;
2-(6,7-Dimethoxy-4-methyl-quinazolin-2-ylamino)-6-(2-dimethylamino-ethoxy)-quinazolin-4-ol trifluoroacetate salt;
2-(6,7-Dimethoxy-4-methyl-quinazolin-2-ylamino)-6-[2-(4-methyl-piperazin-1-yl)-ethoxy]-quinazolin-4-ol trifluoroacetate salt;
2-(6,7-Dimethoxy-4-methyl-quinazolin-2-ylamino)-6-(2-pyrrolidin-1-yl-ethoxy)-quinazolin-4-ol trifluoroacetate salt;
6-(2-Diethylamino-ethoxy)-2-(6,7-dimethoxy-4-methyl-quinazolin-2-ylamino)-quinazolin-4-ol trifluoroacetate salt;
2-[4-Hydroxy-6-(2-morpholin-4-yl-ethoxy)-quinazolin-2-ylamino]-6,7-dimethoxy-quinazolin-4-ol trifluoroacetate salt;
2-(6,7-Dimethoxy-4-methyl-quinazolin-2-ylamino)-6-(3-pyrrolidin-1-yl-propoxy)-quinazolin-4-ol trifluoroacetate salt;
2-(6,7-Dimethoxy-4-methyl-quinazolin-2-ylamino)-6-(2-pyrrol-1-yl-ethoxy)-quinazolin-4-ol trifluoroacetate salt;
2-(6,7-Dimethoxy-4-methyl-quinazolin-2-ylamino)-6-(2-pyrrolidin-1-yl-ethoxy)-quinazolin-4-ol trifluoroacetate salt;
2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(3-hydroxy-piperidin-1-yl)-ethoxy]-quinazolin-4-ol trifluoroacetate salt;
6-{2-[Bis-(2-hydroxy-ethyl)-amino]-ethoxy}-2-(6,7-dimethoxy-4-methyl-quinazolin-2-ylamino)-quinazolin-4-ol trifluoroacetate salt;
2-(6,7-Dimethoxy-4-methyl-quinazolin-2-ylamino)-6-{2-[ethyl-(2-hydroxy-ethyl)-amino]-ethoxy}-quinazolin-4-ol trifluoroacetate salt;
2-(6,8-Dimethoxy-4-methyl-quinazolin-2-ylamino)-6-[2-(3-hydroxy-pyrrolidin-1-yl)-ethoxy]-quinazolin-4-ol trifluoroacetate salt;
2-(6,7-Dimethoxy-4-methyl-quinazolin-2-ylamino)-6-[2-(2-hydroxy-ethylamino)-ethoxy]-quinazolin-4-ol trifluoroacetate salt;
2-(6,7-Dimethoxy-4-methyl-quinazolin-2-ylamino)-6-{2-[(1-hydroxymethyl-3-methyl butyl)-amino]-ethoxy}-quinazolin-4-ol Trifluoroacetate salt;
6-[2-(3-Amino-pyrrolidin-1-yl)-ethoxy]-2-(6,7-dimethoxy-4-methyl-quinazolin-2-ylamino)-quinazolin-4-ol;
2-(6,7-Dimethoxy-4-methyl-quinazolin-2-ylamino)-6-[2-(2-hydroxymethyl-pyrrolidin-1-yl)-ethoxy]-quinazolin-4-ol trifluoroacetate salt;
2-{2-[2-(6,7-Dimethoxy-4-methyl-quinazolin-2-ylamino)-4-hydroxy-quinazolin-6-yloxy]-ethylamino}-propane-1,3-diol trifluoroacetate salt;
2-(6,8-Dimethoxy-4-methyl-quinazolin-2-ylamino)-6-(2-pyrrolidin-1-yl-ethoxy)-quinazolin-4-ol trifluoroacetate salt;
2-(6,8-Dimethoxy-4-methyl-quinazolin-2-ylamino)-6-{2-[ethyl-(2-hydroxy-ethyl)-amino]-ethoxy}-quinazolin-4-ol trifluoroacetate salt;
2-(6,8-Dimethoxy-4-methyl-quinazolin-2-ylamino)-6-[2-(2-hydroxymethyl-pyrrolidin-1-yl)-ethoxy]-quinazolin-4-ol trifluoroacetate salt;
2-(6,7-Dimethoxy-4-methyl-quinazolin-2-ylamino)-6-[2-(2-hydroxy-1-methyl-ethylamino)-ethoxy]-quinazolin-4-ol trifluoroacetate salt;
2-(6,7-Dimethoxy-4-methyl-quinazolin-2-ylamino)-6-[2-(2-hydroxymethyl-morpholin-4-yl)-ethoxy]-quinazolin-4-ol trifluoroacetate salt;
2-(6,7-Dimethoxy-4-methyl-quinazolin-2-ylamino)-6-{2-[(2-dimethylamino-ethyl)-ethyl-amino]-ethoxy}-quinazolin-4-ol trifluoroacetate salt;
2-(6,7-Dimethoxy-4-methyl-quinazolin-2-ylamino)-6-[2-(morpholin-4-ylamino)-ethoxy]-quinazolin-4-ol trifluoroacetate salt;
2-(6,7-Dimethoxy-4-methyl-quinazolin-2-ylamino)-6-[2-(2-hydroxymethyl-piperidin-1-yl)-ethoxy]-quinazolin-4-ol trifluoroacetate salt;
2-(6,7-Dimethoxy-4-methyl-quinazolin-2-ylamino)-6-(2-[1,2,4]triazol-4-yl-ethoxy)-quinazolin-4-ol trifluoroacetate salt;
2-(6,7-Dimethoxy-4-methyl-quinazolin-2-ylamino)-6-{2-[4-(2-hydroxy-ethyl)-piperazin-1-yl]-ethoxy}-quinazolin-4-ol trifluoroacetate salt;
2-(6,7-Dimethoxy-4-methyl-quinazolin-2-ylamino)-6-{2-[N′-(2-hydroxy-ethyl)-hydrazino]-ethoxy}-quinazolin-4-ol trifluoroacetate salt;
2-(6,7-Dimethoxy-4-methyl-quinazolin-2-ylamino)-6-[2-(2,6-dimethyl-morpholin-4-yl)-ethoxy]-quinazolin-4-ol trifluoroacetate salt;
2-(6,7-Dimethoxy-4-methyl-quinazolin-2-ylamino)-6-{N′,N′-dimethyl-hydrazino]-ethoxy}-quinazolin-4-ol trifluoroacetate salt;
2-(6,7-Dimethoxy-4-methyl-quinazolin-2-ylamino)-6-{2-[2-(2-hydroxy-ethoxy)-ethylamino]-ethoxy}-quinazolin-4-ol trifluoroacetate salt;
2-(6,7-Dimethoxy-4-methyl-quinazolin-2-ylamino)-6-[2-(2-pyrrolidin-1-ylmethyl-pyrrolidin-1-yl)-ethoxy]-quinazolin-4-ol trifluoroacetate salt;
2-(6,7-Dimethoxy-4-methyl-quinazolin-2-ylamino)-6-[2-(2-hydroxy-cyclohexylamino)-ethoxy]-quinazolin-4-ol trifluoroacetate salt;
2-(6,7-Dimethoxy-4-methyl-quinazolin-2-ylamino)-6-[2-(methyl-pyridin-3-ylmethyl-amino)-ethoxy]-quinazolin-4-ol trifluoroacetate salt
2-(6,7-Dimethoxy-4-methyl-quinazolin-2-ylamino)-6-[2-(2-pyridin-4-yl-ethylamino)-ethoxy]-quinazolin-4-ol TFA salt; and
2-(6,7-Dimethoxy-4-methyl-quinazolin-2-ylamino)-6-[2-(methyl-pyridin-4-ylmethyl-amino)-ethoxy]-quinazolin-4-ol TFA salt;

5. A pharmaceutical composition comprising a compound of claim 1 or a pharmaceutically acceptable salt of a compound of claim 1.

6. A pharmaceutical composition comprising a compound of claim 4 or a pharmaceutically acceptable salt of a compound of claim 4.

7. A method of treatment, comprising:
a) providing a patient having a bacterial infection; and
b) administering the of compound of claim 1 or a pharmaceutically acceptable salt of a compound of claim 1 to said patient.

8. A method of treatment, comprising:
a) providing a patient having a bacterial infection; and
b) administering compound of compound of claim 4 or a pharmaceutically acceptable salt of a compound of claim 4 to said patient.

9. A method of preparing a bis-quinazoline compound of the formula: 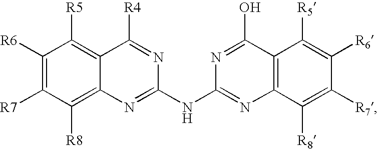
wherein
R₄, and R₄′ are independently selected from the group consisting of hydrogen, alkyl, alkenyl, alkynyl, CH₂-aryl, CH₂-heterocyclic, and phenyl; and
wherein
R₅, R₅′, R₆, R₆′, R₇, R₇′, R₈, and R₈′ are independently selected from the group consisting of hydrogen, halo, nitro, cyano, and hydroxy, C₍₁₋₇₎alkyl (optionally substituted by halo, hydroxy, amino), mono to perfluoro C₍₁₋₃₎alkyl, C₍₃₋₇₎cycloalkyl, C₍₁₋₇₎alkoxy, amino, mono- or di-C₍₁₋₆₎alkylamino, C₍₁₋₇₎alkylthio, benzyloxy, alkenyl, O-alkenyl, S-alkenyl, NH-alkenyl, alkynyl, O-alkynyl, S-alkynyl, NH-alkynyl, CH₂-aryl, CH₂-heterocyclic, aryl, heterocyclic, S-aryl, O-aryl, NH-aryl, phenyl, O-carbohydrate, S-carbohydrate, NH-carbohydrate, sulfamoyl, carbamoyl, N-alkyl fused to aryl or heterocyclic, OCOR₉, SCOR₉, NHCOR₉, NHR₉R₁₀; whereinR₉ and R₁₀ are independently selected from the group consisting of alkyl, aryl, including heterocyclic, and acyl donors selected from the group consisting of alkyl acids, amino acids, and aromatic acids;
and a cyclic ring connecting any of R₅ and R₆; R₆ and R₇; R₇ and R₈; R₅′ and R₆′; R₆′ and R₇′; and R₇′ and R₈′, wherein the cyclic ring is selected from the group consisting of methylenedioxy, dioxane, substituted methylenedioxy, heteroatom five member ring, and heteroatom six member ring;
comprising:
a) providing a salt of an aniline of the formula 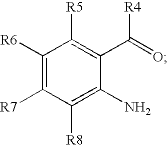
b) reacting it with sodium dicyanamide to generate a quinazolin-2-yl-cyanamide of the formula: 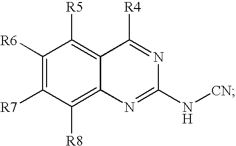
and
c) reacting the quinazolin-2-yl-cyanamide with an alkyl anthranilate of the formula: 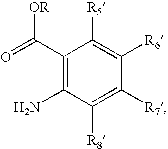
in the presence of a concentrated acid to generate a bis-quinazoline compound of the formula:

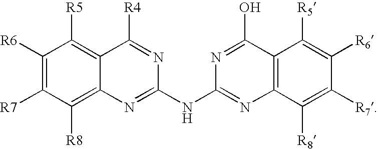

10. The method of claim 9, wherein the concentrated acid is selected from the group consisting of hydrochloric acid, sulfuric acid, and phosphoric acid.

11. A method of preparing a bis-quinazoline compound of the formula: 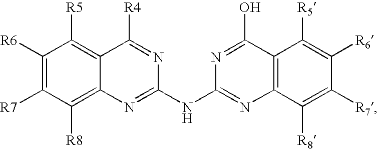
R₄ is independently selected from the group consisting of hydrogen, alkyl, alkenyl, alkynyl, CH₂-aryl, CH₂-heterocyclic, and phenyl; and
wherein
R₅, R₅′, R₆, R₆′, R₇, R₇′, R₈, and R₈′ are independently selected from the group consisting of hydrogen, halo, nitro, cyano, and hydroxy, C₍₁₋₇₎alkyl (optionally substituted by halo, hydroxy, amino), mono to perfluoro C₍₁₋₃₎alkyl, C₍₃₋₇₎cycloalkyl, C₍₁₋₇₎alkoxy, amino, mono- or di-C₍₁₋₆₎alkylamino, C₍₁₋₇₎alkylthio, benzyloxy, alkenyl, O-alkenyl, S-alkenyl, NH-alkenyl, alkynyl, O-alkynyl, S-alkynyl, NH-alkynyl, CH₂-aryl, CH₂-heterocyclic, aryl, heterocyclic, S-aryl, O-aryl, NH-aryl, phenyl, O-carbohydrate, S-carbohydrate, NH-carbohydrate, sulfamoyl, carbamoyl, N-alkyl fused to aryl or heterocyclic, OCOR₉, SCOR₉, NHCOR₉, NHR₉R₁₀; whereinR₉ and R₁₀ are independently selected from the group consisting of alkyl, aryl, including heterocyclic, and acyl donors selected from the group consisting of alkyl acids, amino acids, and aromatic acids;
and a cyclic ring connecting any of R₅ and R₆; R₆ and R₇; R₇ and R₈; R₅′ and R₆′; R₆′ and R₇′; and R₇′ and R₈′, wherein the cyclic ring is selected from the group consisting of methylenedioxy, dioxane, substituted methylenedioxy, heteroatom five member ring, and heteroatom six member ring;
comprising:
a) providing a quinazolin-2-yl guanidine of the formula: 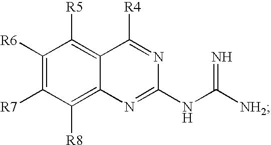
and
b) reacting said quinazolin-2-yl guanidine with an isatoic anhydride of the formula: 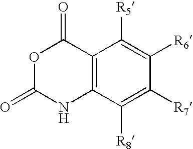
in the presence of a tertiary amine to generate a bis-quinazoline compound of the formula:

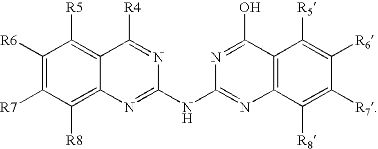

12. The method of claim 11, wherein the tertiary amine is selected from the group consisting of diiopropyethylamine and triethylamine.

13. A method of preparing a bis-quinazoline compound of the formula: 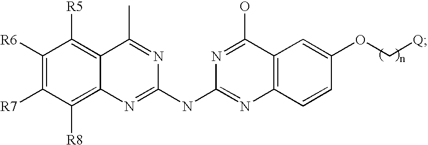
wherein
n is an integer from 1-10; and
wherein
R₅, R₆, R₇, and R₈ are independently selected from the group consisting of hydrogen, halo, nitro, cyano, and hydroxy, C₍₁₋₇₎alkyl (optionally substituted by halo, hydroxy, amino), mono to perfluoro C₍₁₋₃₎alkyl, C₍₃₋₇₎cycloalkyl, C₍₁₋₇₎alkoxy, amino, mono- or di-C₍₁₋₆₎alkylamino, C₍₁₋₇₎alkylthio, benzyloxy, alkenyl, O-alkenyl, S-alkenyl, NH-alkenyl, alkynyl, O-alkynyl, S-alkynyl, NH-alkynyl, CH₂-aryl, CH₂-heterocyclic, aryl, heterocyclic, S-aryl, O-aryl, NH-aryl, phenyl, O-carbohydrate, S-carbohydrate, NH-carbohydrate, sulfamoyl, carbamoyl, N-alkyl fused to aryl or heterocyclic, OCOR₉, SCOR₉, NHCOR₉, NHR₉R₁₀; whereinR₉ and R₁₀ are independently selected from the group consisting of alkyl, aryl, including heterocyclic, and acyl donors selected from the group consisting of alkyl acids, amino acids, and aromatic acids;
and a cyclic ring connecting any of R₅ and R₆; R₆ and R₇; R₇ and R₈; wherein the cyclic ring is selected from the group consisting of methylenedioxy, dioxane, substituted methylenedioxy, heteroatom five member ring, and heteroatom six member ring; and
wherein
Q is selected from the group consisting of phenyl; naphthyl; pyridine; pyrimidine; pyridazine; imidazole; benzimidazole; pyrazole; triazole; furan; thiophene; pyran; naphthyridine; oxazo[4,5-b]pyridine; and imidazo[4,5-b]pyridine; each of which may be optionally substituted with one to three groups selected from the group of halogen, C₁₋₆ alkyl, C₁₋₆ alkoxy, hydroxy, mono- or di-(C₁₋₃ alkyl)amino, C₁₋₆ alkyl-S(O)ₘ and phenylamino, wherein the phenyl ring is optionally substituted with one to two groups from the group halogen, C₁₋₆ alkyl and C₁₋₆ alkoxy;
tetrahydropyran; tetrahydrofuran; 1,3-dioxolanone; 1,3-dioxanone; 1,4-dioxane; morpholine; thiomorpholine; thiomorpholine sulfoxide; thiomorpholine sulfone; piperidine; piperazine; piperidinone; pyrrolidine; tetrahydrpyrimidone; cyclohexanone; cyclohexanol; each of which may be optionally substituted with one to three groups selected from the group consisting of C₁₋₆ alkyl, C₁₋₆ alkoxy, hydroxy, mono- or di-(C₁₋₃ alkyl)amino-C₁₋₃ alkyl, phenylamino-C₁₋₃ alkyl and C₁₋₃ alkoxy-C₁₋₃ alkyl;
C₁₋₆ alkoxy; secondary or tertiary amine wherein the amino nitrogen is covalently bonded to groups selected from the group consisting of C₁₋₆ alkyl, C₁₋₅ alkoxyalkyl, C₁₋₆ branched alkyl, each of which are optionally substituted with halo, hydroxy, or amino;
secondary or tertiary amine wherein the amino nitrogen is covalently bonded to phenyl, C₁₋₂ alkylphenyl or C₁₋₂ alkylpyridyl, wherein the aryl ring is optionally substituted with one to two groups selected from the group consisting of halogen, C₁₋₆ alkoxy, and hydroxyl; comprising:
a) providing a bis-quinazolin-2-yl-amine of the formula: 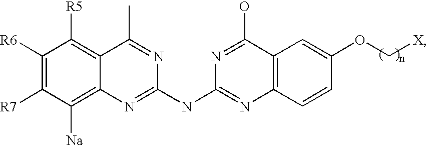
wherein X is selected from the group consisting of Cl, Br, and I;
b) reacting said bis-quinazolin-2-yl-amine with a nucleophile, and
c) heating the resulting mixture to generate a bis-quinazoline compound of the formula:

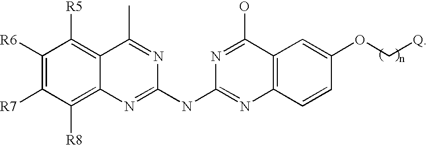

14. The method of claim 13, wherein said nucleophile is selected from the group consisting of a primary amine, a secondary amine, a primary alcohol, a secondary alcohol, a primary alkoxide, a secondary alkoxide, and alkali metal, and an alkali metal hydroxide.
